SEQUENCE LISTING (60 sequences)

SEQ ID NO: 1 - DNA, 1617 bp, Homo sapiens
gtgcctctctatggagagcacctctgtggcctctctgagagcactcacagccaaaagtacacagctgcccccaggctgagagtgcttgatacacccttgaatcccctcttatatgatgccccagcccaggagagataaaagcatcagcaccatgagattcacctgcctctggtcgttagggaacaatggaggcctgcgattggagttaaactctcagtgatctctgtgttgacaacaccaaagctagaggaatccagtaggatgtgggcatggttttcccggaaggctgactgagcagttctgcaaatgtttgcaagtacagggcagaatttcatccagcctcagaaccttgagccaagactcagcatcagcaaagccaaaagtttcatttcttcgactgtgggagtgctagtcccaacctttagatggccattcagttttaagttcaataagcattttgattgagaaatactttgctgaggagtgaaaagtccttggctttgaaagacgaatgatgagcagttcagtggcccatgtcacagtccaggcacctgccaaaggtgactccctgggaggagcatcttagtcacagagccagtgcctgctgtaggtgtgcagaagggtgcatgtgtgtgtgtgtgtgtgtgtgtgtatgtgtacgtgtacatgtgtgttgggggaagggagcaagggttgtgggagcatttcttatctgctcttctctgcaagatttcctgtgatttaagtcacattaaagtacccataagcccgtaatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtacccgcgtgcatatgcgcgcatgcagtgcagggtctgcatacctaaagcagatgaaattctgcagaatggctgcctcgctagacaaagtcaagaagacagaccgaggagagagaggttgatgtgtctccactaccaagagacaggcttctctaagccagcgagacatcccatccaacaatatgaaactggccacatttccttgagatgtcaacgttgaaagtgtagctgcatctttattcttcactgttatgaagttgggtgcaacacagcttgagtggaatacaaaaacaccgcttggaaacacatgatctggatttgaatcgcagctgtatcattcacctgctatagactctgagcaagacctctctgaggttatttcttcacagtaggtagagacaagacttacttcaaaggttcttaaagttgaacctgagtcaatgaatgcaaaagtgttcacatttaaactgtaattttaaagcacaatacaagtaaatagcattaatatcattagagagattaacttagcactgtgcgtcacatgattcatcacggccatctgtgagatatcaaatagagaggtgaagcctgcagtaataaaaaatactgccatagctataaaaaaaaaaaaaaaaaaaaaaaaaaaaaa

SEQ ID NO: 2 - protein, 18 aa, Homo sapiens
RVHMRACSAGSAYLKQMK

SEQ ID NO: 3 - protein, 18 aa, Homo sapiens
GSAYLKQMKFCRMAASLD

SEQ ID NO: 4 - protein, 20 aa, Homo sapiens
FCRMAASLDKVKKTDRGERG

SEQ ID NO: 5 - protein, 18 aa, Homo sapiens
MAASRCSGLYIVRNDTSG

SEQ ID NO: 6 - protein, 18 aa, Homo sapiens
YIVRNDTSGLSGSQWVDS

SEQ ID NO: 7 - protein, 18 aa, Homo sapiens
LSGSQWVDSPLKSPCQVW

SEQ ID NO: 8 - DNA, 9 bp, Homo sapiens
gtgcctctc

SEQ ID NO: 9 - DNA, 535 bp, Homo sapiens
  misc_feature 1, 8, 451, 462, 467, 481, 483, 487, 508, 515, 519, 527,535: n = A,T,C or G
nggaattngtttacgactcactacagggcgaattgggcctctagatgctgctcgagcggccgccagtgtgatggatatctgcagaattcggcttgaacactgcgtttgctggctttgatgaaattaagccacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtgtgtgtgtacccgcgtgcatatgcgcgcatgcagtgcagggtctgcatacctaaagcagatgaaattctgcagaatggctgcctcctanacaaagtcaanaaganagaacgaggagccnantccncacactggcgggcgttactantggatcnaacncggtacanctgaagcn

SEQ ID NO: 10 - DNA, 543 bp, Homo sapiens
  misc_feature 3, 7, 14, 16, 23, 453, 470, 479, 481, 484, 486, 510, 543: n = A,T,C or G
tancgcnaatgctnanttaggtnacactatagaatactcaagctatgcatcaagcttggtaccgagctcggatccactagtaacggccgccagtgtgctggaattcggcttgaacactgcgtttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtgtgtgtgtgtaccgcgtgcatatgcgcgcatgcagtgcagggtctgcatacctaaagcagatgaaattctgcanaatggctgcctcactanacaaagtcnanaananagaacgaggaaccgaattctgcanatatccacacactggggggcgctcgaacagctn

SEQ ID NO: 11 - DNA, 546 bp, Homo sapiens
  misc_feature 1, 3, 4, 9, 15, 18, 47, 53, 501: n = A,T,C or G
ngnnaattngttttnacngactcactgatagggcgaattgggcctcntagtangcatgctcgagcggccgccagtgtgatggatatctgcagaattcggcttcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacacacacacacgggactaggactattccagacttggcagggggactttaatggggagctcaacccactgactgcctgagagcccagaagtgtcatttctgactatctaaagaccagagcatctacttgctgccatggttggctgctgggctggttttggggttcttttgcattatgggcttatgggtactttaatgtgacttaatttcatcaaagccagcaaacgcagtgttcaagccgaattccancacactggcgggcgttactaatggatcgaactcggtacaagctta

SEQ ID NO: 12 - DNA, 540 bp, Homo sapiens
  misc_feature 7, 10, 27, 60, 64, 67, 509, 540: n = A,T,C or G
ggggttnctnatttagtgtccacactnatagaatactcaagctatgcatcaagcttggtnaccnganctcggatccactagtaacggccgccagtgtgctggaattcggctcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacacacacacgggactaggactattccagacttggcagggggactttaatggggagctcaacccactgactgcctgagagcccagaagtgtcatttctgactatctaaagaccagagcatctacttgctgccatggttggctgctgggctggttttggggttcttttgcattatgggcttatgggtactttaatgtggcttaatttcatcaaagccacaaacgcagtgttcaagccgaattctgcanatatccatcacactggcgggcgctcaaactn

SEQ ID NO: 13 - DNA, 542 bp, Homo sapiens
  misc_feature 6, 12, 14, 21, 26, 31, 501, 536, 542: n = A,T,C or G
ttcgcnatgctnanttaggtnacctnatagnaatactcaagctatgcatcaagcttggtaccgagctcggatccactagtaacggccgccagtgtgctggaattcggcttcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacacacacacacacgggactaggactattccagacttggcagggggactttaatggggagctcaacccactgactgcctgagagcccagaagtgtcatttctgactatctaaagaccagagcatctacttgctgccatggttggctgctgggctggttttggggttcttttgcattatgggcttatgggtactttaatgtgacttaatttcatcaaagccacaaaccagtgttcaagccnaattctgcaaatatccacacactggcgggcgctcnaacatn

SEQ ID NO: 14 - DNA, 547 bp, Homo sapiens
  misc_feature 3, 4, 5, 6, 7, 8, 9, 10, 50, 395, 412, 435, 437, 459,463, 466, 467, 468, 476, 481, 492, 499, 501, 504, 510, 516, 519,530, 538, 547: n = A,T,C or G
tannnnnnnnggaatttgtcctacgactcactcgagggcgaattgggcantctatatgctgctcgagcggccgccagtgtgatggatatctgcagaattcggcttgaacactgcgtttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtgtgtgtgtacccgcgtgcatangcgcgcatgcagtgctnggtctgcatacctaaaacaaatnanatctgcagaatgggctgcctcnctanacnnntcaaaaanacgangaaggaaaccnaattccncnccnggcggncgttantantggatccagcncggtaccngctgaagcn

SEQ ID NO: 15 - DNA, 546 bp, Homo sapiens
  misc_feature 3, 14, 16, 21, 31, 443, 474, 485, 488, 529, 538, 546: n = A,T,C or G
tancgcaattgctnanttagngtccacactnatagaatactcaagctatgcatcaagcttggtaccgagctcggatccactagtaacggccgccagtgtgctggaattcggcttgaacactgcgtttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtgtgtgtgtgtacccgcgtgcatatgcgcgcatgcagtgcagggtctgcatacctaaagcanatgaaattctgcagaatggctgcctcactanacaaagtcaanaanacagaacgaggaagcgaattccgcaaatatccaccactggngggcgctcnagcatgcn

SEQ ID NO: 16 - DNA, 536 bp, Homo sapiens
  misc_feature 6, 85, 405, 442, 455, 459, 469, 471, 472, 494, 512, 531: n = A,T,C or G
ggaatngttttacgactcactgcgagggcgaattgggcactctagatgctgctcgagcggccgccagtgtgatggatatctgcanaattcggcttgaacactgcgtttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtgtgtgtgtgtgtacccgcgtgcatatgcgcgcatgcagtgcngggtctgcatacctaaagcagatgaaatctgcagaanggctgctcactanacanagtcaagaananngacgaggaagccgaattccccncctggggggcgttactantggatcgagctcggtacancttga

SEQ ID NO: 17 - DNA, 538 bp, Homo sapiens
  misc_feature 3, 5, 8, 15, 19, 25, 32, 65, 395, 402, 404, 417, 418,422, 437, 442, 444, 461, 465, 467, 470, 472, 489, 497, 513, 516,518, 521, 530, 534, 535, 538: n = A,T,C or G
ttncngcnattgctnattnagggtncacactnatagaatactcaagctatgcatcaagcttgggnaccgagctcggatccactagtaacggccgccagtgtgctggaattcggcttgaacactgcgtttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtgtgtgtgtacncgcgtgnanatgcgcgcatggnntgcngggtctgcatacaanagcananaaaatctgcagaatggntgcncnctnanaagtctaaaaacaaacnaggagccnaaatctgcaaaatccnccncnggnggcgccaanatannctn

SEQ ID NO: 18 - DNA, 548 bp, Homo sapiens
  misc_feature 1, 2, 3, 4, 11, 15, 268, 283, 284, 290, 298, 310, 323,331, 334, 337, 341, 366, 368, 370, 383, 403, 416, 433, 434, 445,453, 458, 461, 463, 466, 469, 472, 485, 500, 512, 517, 524,537, 546, 548: n = A,T,C or G
nnnnggaattngttntacgactcacttatagggcgaattgggcctctagatgcatgctcgagcggccgccagtgtgatggatatctgcagaattcggcttcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacacacacacggggacnacgacaattccacanntggacngggggacnttaatggggagntcaacccactgantgcctganagnccncaantgtcttttctgactatctaaagacnanancatctacttgctnccatggttggctgctgggcngggttttggggtnctcttgcattatgggcnnatgggtatttnatgtgacntaatntcncnaanccnctnacgcagtgttcancccgaattccagcanactgggggcgtnaatantggatcnaactcggtacaancttgatcantn

SEQ ID NO: 19 - DNA, 548 bp, Homo sapiens
  misc_feature 2, 3, 5, 8, 15, 18, 22, 32, 64, 507, 541, 548: n = A,T,C or G
tnncngcnattgctnatntagngtggacactnatagaatactcaagctatgcatcaagcttggntaccgagctcggatccactagtaacggccgccagtgtgctggaattcggcttcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacacacacacacacgggactaggactattccagacttggcagggggactttaatggggagctcaacccactgactgcctgagagcccagaagtgtcatttctgactatctaaagaccagagcatctacttgctgccatggttggctgctgggctggttttggggttcttttgcattatgggcttatgggtactttaatgtgacttaatttcatcaaagccacaacgcagtgttcaagccnaatctgcaaatatccatcacactggcggcgctcnaacatcn

SEQ ID NO: 20 - DNA, 546 bp, Homo sapiens
  misc_feature 531, 535: n = A,T,C or G
ggggggggttttacgactcactgccgagggcgaattgggcactctatatgcatgctcgagcggccgccagtgtgatggatatctgcagaattcggcttcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacacacacacacgggactaggactattccagacttggcagggggactttaatggggagctcaacccactgactgcctgagagcccagaagtgtcatttctgactatctaaagaccagagcatctacttgctgccatggttggctgctgggctggttttggggttcttttgcattatgggcttatgggtactttaatgtgacttaatttcatcaaagccagcaaacgcagtgttcaagccgaattccagcacactggcgggcgttactaatggatcgaactcngtancaagcttgatc

SEQ ID NO: 21 - DNA, 536 bp, Homo sapiens
  misc_feature 1, 2, 4, 5, 10, 12, 13, 274, 335, 360, 365, 420, 468,484, 507, 523, 533: n = A,T,C or G
nncnntgctnanntaggtcacactatagaatactcaagctatgcatcaagcttggtaccgagctcggatccactagtaacggccgccagtgtgctggaattcggcttcctcggtctgtcttcttgactttgtctagtgaggcagccattctgcagaatttcatctgctttaggtatgcagaccctgcactgcatgcgcgcatatgcacgcgggtacacacacacacacacacacacacacacacacacacacacacacacgggactaggactantccagacttggcagggggactttaatggggagctcaacccactgactgcctgagagcccanaagtgtcatttctgactatctaaanaccanagcatctacttgctgccatggttggctgctgggctggttttggggttcttttgcnttatgggcttatgggtactttaatgtgacttaatttcatcaaagccancaaaccagtgttcaanccgaattctgcagatatccacanactggcggcgctccancatgctctanagg

SEQ ID NO: 22 - DNA, 537 bp, Homo sapiens
  misc_feature 2, 7, 391, 412, 444, 447, 448, 455, 458, 468, 482, 483,496, 515, 526, 533, 537: n = A,T,C or G
gnaattngtttacgactcactacagggcgaattgggcctctagatgctgctcgagcggccgccagtgtgatggatatctgcagaattcggcttgaacactgcgtttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtgtgtgtgtgtacccgcgtgcatatgcgcgcatgcagtgcngggtctgcatacctaaagcanatgaaattctgcagaaatggctgcctcactanacnncgtcaanaanacagaccgangaagccgaattccnncccactgggcggncgttactagtggatccaancccggtacaancttgaangcan

SEQ ID NO: 23 - DNA, 535 bp, Homo sapiens
  misc_feature 3, 4, 5, 8, 14, 16, 28, 73, 153, 271, 277, 280, 288,291, 298, 299, 335, 340, 349, 355, 358, 379, 416, 418, 445, 456,465, 483, 489, 508, 526, 532: n = A,T,C or G
tannngcnatgctnanttaggtgaactnatagaatactcaagctatgcatcaagcttggtaccgagctcgganccactagtaacggccgccagtgtgctggaattcggcttcctcggtctgtcttcttgactttgtctagtgaggcagccatnctgcagaatttcatctgctttaggtatgcagaccctgcactgcatgcgcgcatatgcacgcgggtacacacacacacacacacacacacacacacacacacacacacacacacacacncagganaangactattncanacttggnngggggactttaatggggagctcaacccactgactgnctganagcacatangtgtcnttnctgactatctaaagaccagancatctacttgctgccatggttggctgctgggctggtntnggggttcttttgcattatgggcttatnggtactttaangtgacttantttcatcaaagccaacanacgctntgtcaaaccgaatctacanatatccatcacactggcnggcgcncaa

SEQ ID NO: 24 - DNA, 546 bp, Homo sapiens
  misc_feature 1, 2, 3, 316, 408, 429, 432, 443, 460, 482, 483, 487,490, 497, 517, 533, 536, 542, 545, 546: n = A,T,C or G
nnnggaatttgttttacgactcactgcgagggcgaattgggcctctagatgcatgctcgagcggccgccagtgtgatggatatctgcagaattcggcttgaacactgcgttgctggctttgatgaaattaagtcacattaaagtacccataagcccataatgcaaaagaaccccaaaaccagcccagcagccaaccatggcagcaagtagatgctctggtctttagatagtcagaaatgacacttctgggctctcaggcagtcagtgggttgagctccccattaaagtccccctgccaagtctggaatagtcctantcccgtgtgtgtgtgtgtgtgtgtgtgtgtgtgtgtgtgtgtgtgtgtgtgtgtgtgtgtgtacccgcgtgcatatgcgcgcatgcagtgcngggtctgcatacctaaagcanatnaaattctgcanaatggctgcctcactanacaaagtcaaaaaaaaagacgnngaanccnaattccncccctggcgggcgttactantggatccaactcggtncancttgangcnn

SEQ ID NO: 25 - DNA, 29 bp, Artificial Sequence
  oligonucleotide
gcgcaagctttatatgatgccccacccag

SEQ ID NO: 26 - DNA, 31 bp, Artificial Sequence
  oligonucleotide
gcgcggatccgcccgtgatgaatcatgtgac

SEQ ID NO: 27 - DNA, 30 bp, Artificial Sequence
  oligonucleotide
gcgcaagcttcccatgtcacagtccaggca

SEQ ID NO: 28 - DNA, 31 bp, Artificial Sequence; identical to SEQ ID NO: 26
  oligonucleotide

SEQ ID NO: 29 - DNA, 28 bp, Homo sapiens
gcgcaagcttcagccaaccatggcagca

SEQ ID NO: 30 - DNA, 31 bp, Artificial Sequence; identical to SEQ ID NO: 26
  oligonucleotide

SEQ ID NO: 31 - protein, 9 aa, Homo sapiens
MAASRCSGL

SEQ ID NO: 32 - protein, 259 aa, Homo sapiens
  VARIANT 10, 38, 40, 126, 145, 160, 172, 177, 194, 196, 198, 208,219, 221, 224, 227, 241, 245: Xaa = Any Amino Acid
MAASRCSGLXIVRNDTSGLSGSQWVELPIKVPLPSLEXSXSRVCVCVCVCVCVYPRAYARMQCRVCIPKADEILQNGCLARQSQEDRPRRERLMCLHYQETGFSKPARHPIQQYETGHISLRCQRXKCSCIFILHCYEVGCNTAXVEYKNTAWKHMIWIXIAAVSFTCYRLXARPLXGYFFTVGRDKTYFKGSXSXTXVNECKSVHIXTVILKHNTSKXHXYHXRDXLSTVRHMIHHGHLXDIKXRGEACSNKKYCHSY

SEQ ID NO: 33 - protein, 259 aa, Homo sapiens
  VARIANT 12, 94, 122, 157, 184, 211, 213, 235: Xaa = Any Amino Acid
PWQQVDALVFRXSEMTLLGSQAVSGLSSPLKSPCQVWNSPSPVCVCVCVCVCVCTRVHMRACSAGSAYLKQMKFCRMAASLDKVKKTDRGERGXCVSTTKRQASLSQRDIPSNNMKLATFPXDVNVESVAASLFFTVMKLGATQLEWNTKTPLGNTXSGFESQLYHSPAIDSEQDLSEVISSQXVETRLTSKVLKVEPESMNAKVFTFKLXFXSTIQVNSINIIREINLALCVTXFITAICEISNREVKPAVIKNTAIA

SEQ ID NO: 34 - protein, 259 aa, Homo sapiens
  VARIANT 5, 15, 26, 30, 69, 72, 81, 105, 115, 129, 138, 170, 185,201, 218, 229, 248, 252, 253, 258: Xaa = Any Amino Acid
HGSKXMLWSLDSQKXHFWALRQSVGXAPHXSPPAKSGIVLVPCVCVCVCVCVCVPACICAHAVQGLHTXSRXNSAEWLPRXTKSRRQTEEREVDVSPLPRDRLLXASETSHPTIXNWPHFLEMSTLKVXLHLYSSLLXSWVQHSLSGIQKHRLETHDLDLNRSCIIHLLXTLSKTSLRLFLHSRXRQDLLQRFLKLNLSQXMQKCSHLNCNFKAQYKXIALISLERLTXHCASHDSSRPSVRYQIERXSLQXXKILPXL

SEQ ID NO: 35 - DNA, 780 bp, Homo sapiens
accatggcagcaagtagatgctctggtctttagatagtcagaaatgacacttctgggctcccaggcagtcagtgggttgagctccccattaaagtccccctgccaagtctggaatagtcctagtcccgtgtgtgtgtgtgtgtgtgtgtgtgtgtgtgtgtgtacccgcgtgcatatgcgcgcatgcagtgcagggtctgcatacctaaagcagatgaaattctgcagaatggctgcctcgctagacaaagtcaagaagacagaccgaggagagagaggttgatgtgtctccactaccaagagacaggcttctctaagccagcgagacatcccatccaacaatatgaaactggccacatttccttgagatgtcaacgttgaaagtgtagctgcatctttattcttcactgttatgaagttgggtgcaacacagcttgagtggaatacaaaaacaccgcttggaaacacatgatctggatttgaatcgcagctgtatcattcacctgctatagactctgagcaagacctctctgaggttatttcttcacagtaggtagagacaagacttacttcaaaggttcttaaagttgaacctgagtcaatgaatgcaaaagtgttcacatttaaactgtaattttaaagcacaatacaagtaaatagcattaatatcattagagagattaacttagcactgtgcgtcacatgattcatcacggccatctgtgagatatcaaatagagaggtgaagcctgcagtaataaaaaatactgccatagctat

SEQ ID NO: 36 - DNA, 779 bp, Homo sapiens
ccatggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtacccgcgtgcatatgcgcgcatgcagtgcagggtctgcatacctaaagcagatgaaattctgcagaatggctgcctcgctagacaaagtcaagaagacagaccgaggagagagaggttgatgtgtctccactaccaagagacaggcttctctaagccagcgagacatcccatccaacaatatgaaactggccacatttccttgagatgtcaacgttgaaagtgtagctgcatctttattcttcactgttatgaagttgggtgcaacacagcttgagtggaatacaaaaacaccgcttggaaacacatgatctggatttgaatcgcagctgtatcattcacctgctatagactctgagcaagacctctctgaggttatttcttcacagtaggtagagacaagacttacttcaaaggttcttaaagttgaacctgagtcaatgaatgcaaaagtgttcacatttaaactgtaattttaaagcacaatacaagtaaatagcattaatatcattagagagattaacttagcactgtgcgtcacatgattcatcacggccatctgtgagatatcaaatagagaggtgaagcctgcagtaataaaaaatactgccatagctat

SEQ ID NO: 37 - DNA, 778 bp, Homo sapiens
catggcagcaagtagatgctctggtctttagatagtcagaaatgacacttctgggctctcaggcagtcagtgggttgagctccccattaaagtccccctgccaagtctggaatagtcctagtcccgtgtgtgtgtgtgtgtgtgtgtgtgtgtgtgtgtgtacccgcgtgcatatgcgcgcatgcagtgcagggtctgcatacctaaagcagatgaaattctgcagaatggctgcctcgctagacaaagtcaagaagacagaccgaggagagagaggttgatgtgtctccactaccaagagacaggcttctctaagccagcgagacatcccatccaacaatatgaaactggccacatttccttgagatgtcaacgttgaaagtgtagctgcatctttattcttcactgttatgaagttgggtgcaacacagcttgagtggaatacaaaaacaccgcttggaaacacatgatctggatttgaatcgcagctgtatcattcacctgctatagactctgagcaagacctctctgaggttatttcttcacagtaggtagagacaagacttacttcaaaggttcttaaagttgaacctgagtcaatgaatgcaaaagtgttcacatttaaactgtaattttaaagcacaatacaagtaaatagcattaatatcattagagagattaacttagcactgtgcgtcacatgattcatcacggccatctgtgagatatcaaatagagaggtgaagcctgcagtaataaaaaatactgccatagctat

SEQ ID NO: 38 - protein, 183 aa, Homo sapiens
  VARIANT 28, 30, 116, 135, 150, 162, 167: Xaa = Any Amino Acid
IVRNDTSGLSGSQWVELPIKVPLPSLEXSXSRVCVCVCVCVCVYPRAYARMQCRVCIPKADEILQNGCLARQSQEDRPRRERLMCLHYQETGFSKPARHPIQQYETGHISLRCQRXKCSCIFILHCYEVGCNTAXVEYKNTAWKHMIWIXIAAVSFTCYRLXARPLXGYFFTVGRDKTYFKGS

SEQ ID NO: 39 - protein, 241 aa, Homo sapiens
  VARIANT 28, 30, 116, 135, 150, 162, 167, 184, 186, 188, 198,209, 211, 214, 217, 231, 235: Xaa = Any Amino Acid
IVRNDTSGLSGSQWVELPIKVPLPSLEXSXSRVCVCVCVCVCVYPRAYARMQCRVCIPKADEILQNGCLARQSQEDRPRRERLMCLHYQETGFSKPARHPIQQYETGHISLRCQRXKCSCIFILHCYEVGCNTAXVEYKNTAWKHMIWIXIAAVSFTCYRLXARPLXGYFFTVGRDKTYFKGSXSXTXVNECKSVHIXTVILKHNTSKXHXYHXRDXLSTVRHMIHHGHLXDIKXRGEACS

SEQ ID NO: 40 - protein, 198 aa, Homo sapiens
  VARIANT 82, 110, 145, 172: Xaa = Any Amino Acid
SEMTLLGSQAVSGLSSPLKSPCQVWNSPSPVCVCVCVCVCVCTRVHMRACSAGSAYLKQMKFCRMAASLDKVKKTDRGERGXCVSTTKRQASLSQRDIPSNNMKLATFPXDVNVESVAASLFFTVMKLGATQLEWNTKTPLGNTXSGFESQLYHSPAIDSEQDLSEVISSQXVETRLTSKVLKVEPESMNAKVFTFKL

SEQ ID NO: 41 - protein, 239 aa, Homo sapiens
  VARIANT 82, 110, 145, 172, 199, 201, 223: Xaa = Any Amino Acid
SEMTLLGSQAVSGLSSPLKSPCQVWNSPSPVCVCVCVCVCVCTRVHMRACSAGSAYLKQMKFCRMAASLDKVKKTDRGERGXCVSTTKRQASLSQRDIPSNNMKLATFPXDVNVESVAASLFFTVMKLGATQLEWNTKTPLGNTXSGFESQLYHSPAIDSEQDLSEVISSQXVETRLTSKVLKVEPESMNAKVFTFKLXFXSTIQVNSINIIREINLALCVTXFITAICEISNREVKPA

SEQ ID NO: 42 - protein, 240 aa, Homo sapiens
  VARIANT 4, 15, 19, 58, 61, 70, 94, 104, 118, 127, 159, 174, 190,207, 218, 237: Xaa = Any Amino Acid
SQKXHFWALRQSVGXAPHXSPPAKSGIVLVPCVCVCVCVCVCVPACICAHAVQGLHTXSRXNSAEWLPRXTKSRRQTEEREVDVSPLPRDRLLXASETSHPTIXNWPHFLEMSTLKVXLHLYSSLLXSWVQHSLSGIQKHRLETHDLDLNRSCIIHLLXTLSKTSLRLFLHSRXRQDLLQRFLKLNLSQXMQKCSHLNCNFKAQYKXIALISLERLTXHCASHDSSRPSVRYQIERXSLQ

SEQ ID NO: 43 - DNA, 45 bp, Artificial Sequence
  Nucleic acid construct that expresses siRNA
aagtttcattcttcgacttcaagagagtcgaagaaatgaaacttt

SEQ ID NO: 44 - DNA, 46 bp, Artificial Sequence
  Nucleic acid construct that expresses siRNA
gatttcctgtgatttaagttcaagagacttaaatcacaggaaatct

SEQ ID NO: 45 - DNA, 46 bp, Artificial Sequence
  Nucleic acid construct that expresses siRNA
atgatctggatttgaatcttcaagagagattcaaatccagatcatg

SEQ ID NO: 46 - DNA, 49 bp, Artificial Sequence
  Nucleic acid construct that expresses siRNA
aaaagtttcatttcttcgacttcaagagagtcgaagaaatgaaactttt

SEQ ID NO: 47 - DNA, 46 bp, Artificial Sequence; identical to SEQ ID NO: 44
  Nucleic acid construct that expresses siRNA

SEQ ID NO: 48 - DNA, 47 bp, Artificial Sequence
  Nucleic acid construct that expresses siRNA
catgatctggatttgaatcttcaagagagattcaaatccagatcatg

SEQ ID NO: 49 - DNA, 47 bp, Artificial Sequence
  Nucleic acid construct that expresses siRNA
tcattcacctgctatagacttcaagagagtctatagcaggtgaatga

SEQ ID NO: 50 - protein, 19 aa, Homo sapiens
GNNAPAHGVNNAPDNRPAP

SEQ ID NO: 51 - protein, 31 aa, Homo sapiens
MAASRCSGLSEMTLLGSQAVSGLSSPLKSPC

SEQ ID NO: 52 - protein, 32 aa, Homo sapiens
MAASRCSGLRSEMTLLGSQAVSGLSSPLKSPC

SEQ ID NO: 53 - protein, 34 aa, Homo sapiens
MAASRCSGLLDSQKMTLLGSQAVSGLSSPLKSPC

SEQ ID NO: 54 - RNA, 45 nt, Artificial Sequence
  siRNA molecule
aaguuucauucuucgacuucaagagagucgaagaaaugaaacuuu

SEQ ID NO: 55 - RNA, 46 nt, Artificial Sequence
  siRNA molecule
gauuuccugugauuuaaguucaagagacuuaaaucacaggaaaucu

SEQ ID NO: 56 - RNA, 46 nt, Artificial Sequence
  siRNA molecule
augaucuggauuugaaucuucaagagagauucaaauccagaucaug

SEQ ID NO: 57 - RNA, 49 nt, Artificial Sequence
  siRNA molecule
aaaaguuucauuucuucgacuucaagagagucgaagaaaugaaacuuuu

SEQ ID NO: 58 - RNA, 46 nt, Artificial Sequence; identical to SEQ ID NO: 55
  siRNA molecule

SEQ ID NO: 59 - RNA, 47 nt, Artificial Sequence
  siRNA molecule
caugaucuggauuugaaucuucaagagagauucaaauccagaucaug

SEQ ID NO: 60 - RNA, 47 nt, Artificial Sequence
  siRNA molecule
ucauucaccugcuauagacuucaagagagucuauagcaggugaauga

(19) United States
(12) Patent Application Publication
(10) Pub. No.: US 2010/0286243 A1
(43) Pub. Date: Nov. 11, 2010

(54) MIG-7 AS A SPECIFIC ANTICANCER TARGET

(76) Inventor: J. Suzanne Lindsey, Seattle, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 12/738,964

(22) PCT Filed: Oct. 22, 2008
(86) PCT No.: PCT/US08/80829

§ 371 Date: Jul. 12, 2010

Related U.S. Application Data
(60) Provisional application No. 60/981,729, filed on Oct. 22, 2007.

(51) Int. Cl. A61K 31/713; C07H 21/04; A61P 35/00

(52) U.S. Cl. 514/44 A; 536/24.5

(57) ABSTRACT

Aspects of the present invention provide novel Mig-7 encoding nucleic acids and Mig-7 polypeptides, RNAi (such as siRNA), recombinant DNA expression systems and host cells containing same, as well as methods of inhibiting expression of the subject nucleic acid molecules, inhibiting production of the encoded proteins or polypeptides, inhibiting metastasis of a carcinoma cell in a subject (including in humans), inhibiting migration/invasion of and mimicking of normal cells by carcinoma cells in a subject, detecting the presence of a cancer cell (e.g., a migrating/invading cancer cell or carcinoma cell mimic, tumor neovas-cularization, and/or vascular mimicry) in a sample of a subject's tissue or body fluids, and inhibiting the migration/invasion of an endothelial cell mimicking by a placental cell into the blood stream or vessels of a female mammal. Particular aspects relate to novel anti-Mig-7 antibodies, diagnostic and/or prognostic methods, and therapeutic methods comprising use of the inventive nucleic acids, polypeptides and antibodies or derivatives thereof.

### CROSS-REFERENCE TO RELATED APPLICATION

This application is a U.S. National Phase Application of International Application No. PCT/US2008/080829, filed Oct. 22, 2008; which claims the benefit of U.S. Provisional Patent Application No. 60/981,729 filed Oct. 22, 2007 under 35 U.S.C. §119(e). These applications are incorporated herein by reference in their entirety.

### STATEMENT OF GOVERNMENT INTEREST

This invention was made with government support under grant number CA93925 awarded by National Institutes of Health. The government may therefore have certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 790097_{—}403PC_SEQUENCE_LISTING.txt. The text file is 42 KB, was created on Oct. 22, 2008, and is being submitted electronically via EFS-Web, concurrent with the filing of the specification.

### BACKGROUND

1. Technical Field

Aspects of the present invention generally relate to Mig-7-based cancer therapeutics and methods of using the same. More specifically, the present invention provides agents that target Mig-7 nucleic acids and Mig-7 polypeptides in order to inhibit carcinoma cell metastasis and carcinoma cell migration and/or invasion.

2. Description of the Related Art

Migration Inducting Gene 7 (Mig-7) Migration Inducting Gene 7 (Mig-7) is a carcinoma-specific gene that is tightly regulated during transcription and translation (Crouch et al. Experimental Cell Research 2004; 292(2):274-87; Phillips and Lindsey. Oncology Reports 2005; 13:37-44).

Mig-7 is a cysteine-rich protein localized to the cell membrane and cytoplasm whose mRNA and protein synthesis is atypical (Crouch et al. Experimental Cell Research 2004; 292(2):274-87; Phillips and Lindsey. Oncology Reports 2005; 13:37-44). Mig-7 is transcribed from an unusual transcription unit; Thus, gene prediction algorithms have not been effective in predicting the open reading frame (ORF) of Mig-7. As a result, neither Mig-7 nor homologous Mig-7 ESTs are currently represented on any microarray even though its expression is restricted to carcinoma cells. Mig-7 is expressed as a result of receptor tyrosine kinase (RTK) activation in concert with ligation of αvβ5 integrin (αvβ5). Mig-7 antisense olignucleotide (ODN) but not inverted antisense ODN inhibits carcinoma cell scattering (Crouch et al. Experimental Cell Research 2004; 292(2):274-87). Malignant tumors, blood from cancer patients, and cancer cell metastases express Mig-7 regardless of tissue origin. Notably, Mig-7 has not been detected in 25 different normal tissues (n=6 each tissue) or in blood from normal subjects (Crouch et al. Experimental Cell Research 2004; 292(2):274-87; Phillips and Lindsey. Oncology Reports 2005; 13:37-44).

Laminin 5 γ2 promigratory fragments promote aggressive, invasive tumor cell (e.g., melanoma cells) formation of vessel-like structures in 3-D cultures. In vivo, tumor cells, rather than endothelial cells, form vessels in the interior, more hypoxic region of tumors (Hendrix et al. Ann NY Acad Sci 2003 May 1; 995(1):151-61.). Moreover, RTK-induced cancer cell migration, invasion, and dissemination of aggressive carcinoma cells requires αvβ5 signaling (Brooks et al. Journal of Clinical Investigation 1997; 99(6):1390-8; Klemke et al., Journal of Cell Biology 1994; 127(3):859-66); the crosstalk that induces Mig-7 expression, in vivo and in vitro (Crouch et al. Experimental Cell Research 2004; 292(2):274-87). Fetal cytotrophoblasts are similar to cancer cells because they invade the maternal tissues during early placental development under RTK and αvβ5 signaling, evade immune system detection, endovascularly invade and are the only other cell type known to undergo vasculogenic mimicry (Folberg et al., Am J Pathol 2000; 156(2):361-81). Surprisingly, Mig-7 ESTs were isolated from early invasive stage placenta as well as all cancer types studied. In contrast, Mig-7 expression is not detected in noninvasive term placenta or in other normal tissues tested (Crouch et al. Experimental Cell Research 2004; 292(2):274-87; Phillips and Lindsey. Oncology Reports 2005; 13:37-44). Moreover, Mig-7 is expressed by fetal cytotrophoblasts as well as carcinoma cells and plays a role in their common cell behavior of vessel-like structure formation and vascular remodeling. As presently disclosed herein, adhesion assays to various components of the extracellular matrix suggests that a mechanism for Mig-7 in tumor cell vessel formation is due to less adhesion to laminin.

There is a pronounced need in the art for compositions and methods to improve the specificity of cancer treatments and to decrease non-specific toxic side effects. There is also a pronounced need for discovery and targeting of solid cancer-specific gene expression, particularly broad-spectrum or pan-carcinoma expression, to provide for improved dosing and fewer side effects.

### BRIEF SUMMARY

Because Mig-7 expression is specific to solid cancer cells in the adult and plays a role in aggressive tumor cell behavior, the present Applicant conceived that Mig-7 expression provides for a cancer-specific target allowing for novel diagnostic/prognostic and/or therapeutic compositions and methods that will inhibit the spread of cancer while preventing damage to normal cells.

Thus, Mig-7 is a pan-solid cancer cell target, and targeting Mig-7 in anticancer therapies provides novel therapies specific to tumor cells while sparing normal cells, and enabling higher effective doses with fewer side-effects. Further, Mig-7 is a pan-carcinoma expression marker for cancer diagnosis and/or prognosis, as well as a pan-carcinoma therapeutic target for cancer therapies.

In one aspect, the present invention provides for a composition comprising at least one siRNA expressed from a construct comprising a sequence selected from the group consisting of: SEQ ID NOs: 43-49. The present invention also provides for the use of synthesized siRNAs according to SEQ ID NOs: 54-60.

In particular aspects, the siRNA is double stranded.

In another aspect, the present invention provides a method for inhibiting Mig-7 expression in a cell comprising contacting said cell with an effective amount of Mig-7 inhibitor, wherein said inhibitor comprises siRNA.

In particular aspects, the contacted cell is located in a subject. In related aspects, the subject is a human.

The present invention also provides a method for inhibiting cancer cell proliferation, invasion, vascular mimicry, metastasis, or tumor cell growth comprising contacting said cancer cell with a composition comprising an effective amount of at least one Mig-7 inhibitor, wherein at least one said inhibitor comprises siRNA.

In another aspect, the present invention provides a method for treating cancer in a subject, comprising administering to said subject a composition comprising an effective amount of at least one Mig-7 inhibitor, wherein at least one said inhibitor comprises siRNA.

In yet another aspect, the present invention provides a method for reducing the size of a tumor in a subject, comprising administering to said subject a composition comprising an effective amount of at least one Mig-7 inhibitor, wherein at least one said inhibitor comprises siRNA.

In a related aspect, the present invention provides a method for inhibiting cancer cell proliferation, invasion, vascular mimicry, metastasis, or tumor cell growth comprising contacting said cancer cell with a composition comprising an effective amount of at least one Mig-7 inhibitor, wherein at least one said inhibitor comprises siRNA, and wherein the composition further comprises an effective concentration of at least one Mig-7 antibody.

In another aspect, the present invention provides a method for treating cancer in a subject, comprising administering to said subject a composition comprising an effective amount of at least one Mig-7 inhibitor, wherein at least one said inhibitor comprises siRNA, and wherein the composition further comprises an effective concentration of at least one Mig-7 antibody.

In yet another aspect, the present invention provides a method for reducing the size of a tumor in a subject, comprising administering to said subject a composition comprising an effective amount of at least one Mig-7 inhibitor, wherein at least one said inhibitor comprises siRNA, and wherein the composition further comprises an effective concentration of at least one Mig-7 antibody.

The present invention provides for a method to reduce matrix metalloprotease, ERK1/2, Akt and S6 kinase activity in a cancer cell, comprising contacting the cell with a composition comprising an effective amount of at least one Mig-7 inhibitor, wherein at least one said inhibitor comprises siRNA.

In particular aspects, the composition further comprises an effective concentration of at least one Mig-7 antibody.

In other aspects, the present invention also provides for methods to detect breast cancer. Thus, in one aspect, the present invention provides for a method for detecting the presence of a breast cancer in a subject, comprising: obtaining a sample from the patient; contacting the sample with a nucleic acid probe; detecting the presence or absence of Mig-7 mRNA in the sample, wherein the presence of Mig-7 mRNA indicates the presence of breast cancer in the patient.

In a related aspect, the present invention provides a method for detecting the presence of a breast cancer in a subject, comprising: obtaining a sample from the patient; contacting the sample with a binding agent; detecting the presence or absence of Mig-7 protein in the sample, wherein the presence of Mig-7 protein indicates the presence of breast cancer in the patient.

In particular aspects, methods of detecting breast cancer according to the present invention include a sample that is a tissue sample from breast tissue, blood, sputum, serum, or urine.

In other aspects, methods of detecting breast cancer according to the present invention further comprise a step of comparing the expression of Mig-7 in the sample to a control

### BRIEF DESCRIPTION OF THE SEVERAL VIEWS OF THE DRAWINGS

FIGS. 1A and 1B show, according to exemplary aspects of the present invention, that antisense to Mig-7 inhibits chemoinvasion. (A) Immunoblot demonstrating that Mig-7 antisense ODN decreases Mig-7 protein levels in lysates from HEC1A cells treated as described in Materials and Methods, as compared to lysates from HEC1A cells treated with Mig-7 sense ODN, (B) Mig-7 antisense (as), significantly inhibits HEC1A carcinoma cell SF (HGF) chemoinvasion of Matrigel as compared to SF (HGF) treatment alone or SF (HGF) with sense (se) Mig-7 ODN. Images below each treatment group show a representative invasion of cells for that group. Experiments were performed in triplicate for each treatment and repeated three times.

FIG. 2 shows, according to exemplary aspects of the present invention, that antibody specific to Mig-7 inhibits HEC1A endometrial carcinoma chemoinvasion. Mig-7 affinity purified antibody but not IgG isotype antibody significantly decreased chemoinvasion of HEC1A cells into Matrigel. Experiments were performed in triplicate for each treatment and repeated three times.

FIGS. 3A-D show, according to exemplary aspects of the present invention, that Mig-7 peptides enhance human monocyte killing of MCF-7 breast carcinoma cells. (A) Representative RT-PCR demonstrating expression of Mig-7 in MCF-7 cells. (B) Human MC cells stimulated with IL-2 and either no peptide (O), pooled irrelevant peptides (IR), or pooled Mig-7 peptides. (see Table 1). Note that Mig-7 peptides significantly (p value 0.001) enhanced MC killing of MCF7 carcinoma cells. Experiments have been repeated 3 times in replicates of 6 for each treatment group. MC were isolated from two different individuals. (C) Cytotoxic response of human isolated MC after peptide stimulation using indicated ratios of immune cells (MC) to MCF-7 cells. Bars are ±S.E. Experiments were repeated twice with replicates of six for each treatment group. (D) TNF-α production by human isolated MC after indicated peptide stimulation. TNF-α production was measured by ELISA assay as described in Methods and Materials after MC were cultured for 8 days with MUC1 peptide plus irrelevant peptides Mig-7 peptides. Bars are ±S.E. Assays were performed in triplicate.

FIG. 4 (A-C) shows, according to exemplary aspects of the present invention, 16 sequences (SEQ ID NOS:9-24) corresponding to representative sequences of Mig-7 region containing gt repeats (underlined) ranging from 23 to 29 repeats in length. The sequences were not derived using the genetically engineered E. coli (SURE™, Stratagene) that was determined herein to provide for maintaining the integrity of the Mig-7 purine-pyrimidine repeat coding region.

FIGS. 5A and 5B show, according to exemplary aspects of the present invention, that genetically engineered E. coli (SURE™, Stratagene) are required (suitable) to maintain the integrity of the Mig-7 purine-pyrimidine repeat coding region. (A) shows representative sequences of Mig-7 region containing gt repeats (underlined) ranging from 23 (SEQ ID NO: 22) to 29 (SEQ ID NO: 24) repeats in length (additional sequences are shown in FIG. 4). (B) shows representative sequences of Mig-7 plasmids grown in SURE cells. Note the consistent number (18) of gt repeats.

FIG. 6A shows Mig-7 3XFLAG-CMV constructs according to exemplary aspects of the present invention. FIG. 6B shows that Mig-7 cloning into 3XFLAG-CMV produces protein from the Kozak consensus site ATG and not from other upstream ATG sites.

FIGS. 7A and 7B show, according to exemplary aspects of the present invention, that the Mig-7 sequence that produced protein in vivo contains multiple stop codons in reading frame “0”. Plasmids were produced and grown as previously described in FIG. 5, then sent to two different sequencing facilities, Texas Tech University Sequencing Core and Sequetech, Inc. Both facilities sequenced at least twice using two different overlapping primer sets in both directions each. (A) Representative sequencing scheme of overlapping and complementary strand sequencing. (B) shows that the consensus sequences from each facility are 100% homologous. Stop codons are indicated with asterisks in the predicted amino acid sequence from reading frame “0”. Green highlights are UGA that can also encode for selenocysteine. Red highlights are UAG or UAA stop codons.

FIGS. 8A-8E show, according to exemplary aspects of the present invention, that the Mig-7 sequence contains a predicted shift-site and pseudoknot indicating potential frame-shifting, and that Mig-7 protein levels are increased with polyamine treatment consistent with recoding during translation.

FIGS. 9A, 9B and 9C show, according to exemplary aspects of the present invention, and in view of the proposed shift −1 or +1 in reading frame at slippery or “shifty” sites (e.g., a shift of −1 or +1 in the reading frame after the ninth amino acid of Mig-7), three potential reading frames (−1, 0, +1) for the corresponding Mig-7 encoding sequences (SEQ ID NO:35, 36 and 37). (A) shows the 0-frame, (B) shows the +1-shifted frame, and (C) shows the −1-shifted frame.

FIG. 10 shows, according to exemplary aspects of the present invention, that Mig-7 localizes primarily to vessels in xenograft nude mouse models of metastasis. Representative confocal analysis of Mig-7 protein immunohistochemical localization is shown. Rhodamine-conjugated anti-rabbit IgG (red) bound to Mig-7 antibody, which recognizes the first nine amino acids of Mig-7 (e.g., MAASRCSGL), was overlayed on the same area and the sample scanned sequentially for Fluoroscein-conjugated anti-mouse IgG (green) binding to β-actin antibody. Arrows point to Mig-7 positive areas of vessels in lymph nodes to which subcutaneously injected HEC1A endometrial carcinoma cells in Matrigel had spread.

FIGS. 11A-11C show, according to exemplary aspects of the present invention, that RTK ligands, IGF-1 and EGF, known to be produced in the tumor microenvironment, induce Mig-7 in αvβ5 integrin-positive carcinoma cells but not in αvβ5 negative HT29 cells. (A) shows a representative Western blot analysis of integrin β5 expression by HT29, FG and RL95 cells. HT29 are negative while FG and RL95 are positive for β5. (B) shows a representative Northern blot analysis demonstrated that EGF and IGF-1 induce Mig-7 expression in β5+FG pancreatic carcinoma cells. RL95 cells express β5 and Mig-7. (C) shows that relative RT-PCR revealed that HT29 cells do not express Mig-7 mRNA. Each experiment was performed 3 times with similar results.

FIGS. 12A-D show, according to exemplary aspects of the present invention, that Mig-7 is expressed by invading, first and second trimester fetal cytotrophoblasts.

FIGS. 13A-C show, according to exemplary aspects of the present invention, that Mig-7 expression causes decreased adhesion to laminin and was sufficient to cause vessel formation in Matrigel 3-D cultures. (A) shows that HT29 cells transfected with 3XCMVFLAG-Mig-7 expression vector express the same sized protein detected by anti-FLAG antibody as does the affinity purified Mig-7 antibody of endogenous Mig-7 in HEC1A cell lysates. Cells were plated on Matrigel for 17, 48 and 72 hours. Empty vector transfected HT29 cells are negative for FLAG Mig-7 as are parental HT29. (B) shows that HT29 Mig-7 expressing cells are >30% less adherent to a mix of laminins 1, 2, 3, 6, 8 & 10 in a statistically significant manner (p<0.001). (C) shows that HT29 cells with empty vector form discrete colonies in 3-D Matrigel cultures (left panel). In contrast, HT29 cells expressing FLAG Mig-7 invade and form vessel structures (right panel and inset, supplemental data). All experiments were performed at least twice (3-D cultures three times) in quadruplicate.

FIG. 14A shows a reduction in Mig-7 protein levels in samples expressing siRNA (1-3A or 3-1A) (SEQ ID NOs: 43 and 45, respectively) compared to control (parental) cells.

FIG. 14B illustrates the reduction in tumor size in vivo with cells expressing siRNA to Mig-7 (1-3A and 3-1A) (SEQ ID NOs: 43 and 45, respectively) versus control (parental) cells.

FIG. 15 shows that (A) Mig-7 affinity-purified antibody, but not IgG rabbit isotype antibody, significantly decreased chemoinvasion of HEC1A cells into Matrigel toward HGF (p<0.01). Ab=antibody, w/HGF=bottom wells contained 20 ng/ml HGF. Results shown are after 72 hours of invasion. Results are representative of three independent experiments performed in triplicate. Bars represent SEM. (B) Representative flow cytometry histograms of control normal rabbit IgG and Mig-7 antibody-treated HEC1A cells stained with YO-PRO-1. YO-PRO-1 single positive cells (lower oval) are apoptotic, and double positive cells (upper oval) are dead. Mean percentages of three samples are shown. (C) Representative flow cytometry histograms of control IgG and Mig-7 antibody-treated HEC1A cells stained with propridium iodide (PI). Cells in G2/M phase are indicated on histograms. Mean percentages of three samples are shown. Flow cytometry experiments were conducted twice with similar results.

FIG. 16 shows that Mig-7 specific antibody or expression of siRNA specific to Mig-7 decreases activity of MT1-MMP. (A) Representative immunoblot and densitometry results for α2-macroglobulin capture assay of active MT1-MMP in lysates from equal numbers of HEC1A cells treated with normal rabbit IgG or with Mig-7 antibody. All samples were run on the same gel. Mig-7 antibody treatment decreased MT1-MMP activity by 54% compared to IgG treatment. Densitometry data are for the upper, captured MT1-MMP band in each lane normalized to its respective tubulin band. Bars represent SEM. (B) Densitometry analysis of the upper, captured MT1-MMP band from α2-macroglobulin capture assay in lysates from RL95 cells stably transfected with siRNA 1-3 (SEQ ID NO: 46) (decreased Mig-7 expression) and 3-1 (SEQ ID NO: 48) (endogenous levels of Mig-7 expression). Results are normalized to β-tubulin. RL95 cells expressing siRNA 1-3 (SEQ ID NO: 46) showed a 57% decrease in MT1-MMP activity compared to 3-1 (SEQ ID NO: 48) expressing cells. Bars represent SEM. (C) Representative flow cytometry histograms of RL95 siRNA 1-3 (SEQ ID NO: 46) and 3-1 (SEQ ID NO: 48) stably transfected cells stained with YO-PRO-1. YO-PRO-1 single positive cells (lower oval) were apoptotic, and double positive cells (upper oval) were dead. Mean percentages of three samples are shown. (D) Representative flow cytometry histograms of RL95 siRNA 1-3 (SEQ ID NO: 46) and 3-1 (SEQ ID NO: 48) stably transfected cells stained with propidium iodide (PI) for cell cycle analysis. Cells in G2/M phase are indicated on histograms. Mean percentages of three samples are shown. Flow cytometry experiments were conducted twice with similar results.

FIG. 17 shows that expression of siRNA specific to Mig-7 decreases S6 kinase, ERK1/2 and Akt phosphorylation in RL95 cells. (A) Normalized fluorescence intensity from analysis of phosphorylated ERK1/2, Akt and S6 kinase in RL95 siRNA 1-3 (SEQ ID NO: 46) and 3-1 (SEQ ID NO: 48) stably transfected cells. 1-3 (SEQ ID NO: 46) expressing cells in which levels of Mig-7 are significantly reduced by >50% showed a 40% decrease (p<0.01) in ERK1/2 activation and a 10% decrease (p<0.05) in Akt activation compared to 3-1 (SEQ ID NO: 48) expressing RL95 cells that express levels of Mig-7 similar to the RL95 parental cell line. (B) Normalized fluorescence intensities from analysis of phosphorylated PRAS40 and IGF-1 R. All phosphorylation fluorescence intensities were normalized to total respective protein fluorescence intensities. These experiments were performed in triplicate.

FIG. 18 shows that Mig-7 expression is specific to breast carcinoma tissue and cells. (A) Representative Mig-7 specific relative RT-PCR of three breast carcinoma cell lines (T47D, MDA-MB453, DU4475) and normal breast tissue from three subjects without previous history of cancer. (B-D) Representative images of Mig-7 antibody IHC on breast tissue array from Cybrdi, Inc. Core samples from (B) breast carcinoma. Arrows point to positive Mig-7 staining. (C) Representative normal breast tissue IHC with Mig-7 antibody. (D) Representative image of control normal rabbit IgG instead of Mig-7 as primary antibody IHC of breast carcinoma tissue section (serial section to that shown in B). Hematoxylin was used to counterstain. Note a lack of specific staining in (D) compared to (B). Images were taken with 10× objective on a Nikon microscope (100× total magnification) and inserts with 40× objective (400× total magnification). Scale bars indicate 100 μm for 100× images and 20 μm for inserts.

FIG. 19 shows that stable knockdown of Mig-7 expression in RL95 cells decreases early primary tumor growth in nude mice. (A) Representative immunoblot and densitometry of RL95 parental and Mig-7-specific siRNA stably transfected pooled clones 1-3 (SEQ ID NO: 46), 3-1 (SEQ ID NO: 48), and 4-2 (SEQ ID NO: 49) cell lines. Cells were grown for 4 days before harvest. Reprobing with β-tubulin was used as a loading control. Mig-7 levels were reduced in 1-3 (SEQ ID NO: 46) and 4-2 (SEQ ID NO: 49) expressing cells by 43% and 30%, respectively, compared to 3-1 (SEQ ID NO: 48) expressing cells. Statistical analyses of 1-3 (SEQ ID NO: 46), 3-1 (SEQ ID NO: 48) and parental Mig-7 knockdown and sequences of specific siRNAs were shown previously. (B) Representative graph showing the tumor volume (mm³) measured 11, 13, 15, 18, and 23 days after injection of RL95 parental cells or cells stably transfected with siRNA constructs 1-3 (SEQ ID NO: 46), 3-1 (SEQ ID NO: 48), or 4-2 (SEQ ID NO: 49) into nude mice. Cells expressing siRNAs 1-3 (SEQ ID NO: 46) and 4-2 (SEQ ID NO: 49) showed 60% and 40-50% (p<0.05) decreased tumor volume, respectively, at days 13 and 15. Bars represent SEM. Experiment was performed in triplicate, n=5 mice for each treatment group, with similar results.

### DETAILED DESCRIPTION

### Definitions

As used herein, the term “protective immunity” refers to the art-recognized protective immunity by a host, the immunity having been induced within the host by one or more prior vaccinations, or by one or more prior pathogen infections.

As used herein, the terms “passive immunity” or “immediate immunity” refers to the immunity conferred within a host, by passive antibody administration, wherein, passive antibody can theoretically confer protection regardless of the immune status of the host. Passive antibody administration can be used for post-exposure prophylaxis.

As used herein, the term “epitope” refers to, as is known in the art, an antigenic determinant of a protein of polypeptide. An epitope could comprise 3 amino acids in a spacial conformation which is unique to the epitope. Generally an epitope consists of at least 5 such amino acids. An epitope of a polypeptide or protein antigen can be formed by contiguous or noncontiguous amino acid sequences of the antigen. A single viral protein, for example, may contain many epitopes. Additionally, a polypeptide fragment of a viral protein may contain multiple epitopes. The present invention encompasses epitopes and/or polypeptides recognized by antibodies of the present invention, along with conservative substitutions thereof, which are still recognized by the antibodies. Further truncation of these epitopes may be possible.

As used herein, the term “ELISA” refers to enzyme-linked immunosorbent assays, as widely recognized in the art, and as described herein.

As used herein, the term “immunologic assay,” refers to an art-recognized immunologic assay suitable to detect the formation of antigen:antibody complexes, including, but not limited to antibody capture assays, antigen capture assays, and two-antibody sandwich assays, ELISA, immunodiffusion, immunoelectrophoresis, immunochemical methods, binder-ligand assays, immunohistochemical and immuncytochemical techniques, Western analysis, agglutination and complement assays (see e.g., Basic and Clinical Immunology, 217-262, Sites and Terr, eds., Appleton & Lange, Norwalk, Conn., 1991 which is incorporated herein by reference). Preferred aspects (e.g., ELISA) of such assays are described herein below. According to the present invention, one or more of such immunoassays can be used to detect and/or quantitate antigens (e.g., Harlow & Lane, Antibodies: A Laboratory Manual; Cold Spring Harbor Laboratory, New York 555-612, 1988, incorporated by reference herein).

As used herein, the term “treatment” refers to both therapeutic treatment and prophylactic or preventative measures, wherein the object is to prevent or slow (lessen) cancer, pathogens (e.g., viral) infection or associated conditions. Those in need of treatment include those already experiencing a condition or infection, those prone to the condition or infection, and also those in which the potential condition or infection is to be prevented.

As used herein, the term “antibodies,” refers to the art-recognized definition, described in more detail herein below.

As used herein, the term “neutralizing antibodies,” refers to the art-recognized definition.

As used herein, the term “cognate antigen,” refers to an antigen that is specifically bound by a cognate antibody, and “cognate antibody” refers to the antibody that specifically binds a cognate antigen.

As used herein, the term “vaccine,” refers to any type of biological agent in an administratable form capable of stimulating an immune response in an animal inoculated with the vaccine. For purposes of preferred aspects of this invention, an inventive vaccine may comprise as the Mig-7 polypeptide agent, one or more immunogenic (antigenic) components of Mig-7, and including polypeptide-based vaccines.

### Migration Inducting Gene 7 (Mig-7)

Treating cancer has been hampered due to toxicity and other adverse side effects. As a result, cancer-specific, instead of overexpressed targets are needed. Carcinoma cells and fetal trophoblasts express a novel gene called Migration inducting gene-7 (Mig-7) that is recoded from mRNA containing multiple stop codons, and therefore, is not represented on any microarray. Growth factors (e.g., epidermal, hepatocyte and insulin-like 1 growth factors) that are overexpressed in the tumor microenvironment, induce expression of Mig-7 in αvβ5 integrin-positive cancer cells. Receptor tyrosine kinase activation and αvβ5 integrin signaling are known to be required for tumor cell dissemination in vivo. Mig-7 expression mediates tumor cell detection of the microenvironment, invasion and vessel-like structure formation in 3-D cultures.

Mig-7 expression is specific to carcinoma cells in the adult and plays a role in aggressive tumor cell behavior; thus, Mig-7 expression provides for a cancer-specific target and allows for novel diagnostic/prognostic and/or therapeutic compositions and methods that will inhibit the spread of cancer while preventing damage to normal cells.

According to particular aspects of the present invention, Mig-7 is a pan-carcinoma cell target, and targeting Mig-7 in anticancer therapies provides novel therapies specific to tumor cells while sparing normal cells, and enabling higher effective doses with fewer side-effects.

According to additional aspects of the present invention, Mig-7 is a pan-carcinoma expression marker for cancer diagnosis and/or prognosis, as well as a pan-carcinoma therapeutic target for cancer therapies.

Art-recognized in vitro model systems designed to assay cell invasiveness and tumor cell killing were employed herein to confirm Mig-7 is a pan-carcinoma cell target, and a pan-carcinoma expression marker for cancer diagnosis and/or prognosis.

For example, Mig-7 specific antisense constructs or antibodies significantly reduced chemoinvasion by >60% in an HEC1A cell chemoinvasion assay. Additionally, breast carcinoma cell killing was significantly enhanced by stimulating isolated, human monocyte cells with Mig-7 peptides in vitro. Furthermore, stably expressing Mig-7-specific short interfering RNA significantly reduced Mig-7 protein levels, chemoinvasion, and early primary tumor growth in a xenograft nude mouse model. Without wishing to be bound to any particular theory, reduced phosphorylation of ERK1/2, Akt, and S6 kinase as well as decreased membrane-type 1 matrix metalloproteinase activity were among mechanisms through which Mig-7 protein was shown to cause these effects. Thus, the present invention contemplates, in part, that Mig-7 would be a suitable, and in some instances a preferred target for cancer therapy.

Example 1 described herein demonstrates the efficacy and importance of targeting Mig-7, both in vitro and in vivo. Targeting mRNA with antisense oligonucleotides or with RNAi inhibits translation and downstream functions of overexpressed tumor cell proteins and has been shown to be a viable therapy to decrease tumor progression (Ahmad et al. Anti-cancer Drugs 2006; 16(10):1037-43; Curcio et al. Pharmacology Therapeutics 1997; 74:317-32; Elez et al. Oncogene 2003; 22(1):69-80; Wang et al. International Journal of Oncology 2002; 21(1):73-80; Zelweger et al. Journal of Pharmacology & Experimental Therapeutics 2001; 298(3):934-40). However, to date, none of these targets are cancer-specific and ultimately in vivo toxicity as well as negative effects on normal cells occurs. Thus, the present invention contemplates, in part, that it is preferred to target carcinoma specific genes, such as Mig-7. Targeting Mig-7 would allow higher, more effective doses; and moreover, because of Mig-7's cancer cell specific expression, Mig-7 antagonists (e.g., antibodies, siRNAs or antisense etc.) would not affect normal cells.

Additionally, anti-cancer antibody therapies are one of the newest targeting approaches. For example, Herceptin® (Trastuzumab), an antibody therapy, targets HER2, an EGF receptor that is overexpressed in approximately 20% of breast cancer patients. After a median follow-up of 23 months in clinical trials of over 3,000 breast cancer patients positive for HER2 overexpression, 13.7% of women treated with doxorubicin and cyclophosphamide then by docetaxel suffered recurrence or death, compared with 7.2% of women treated with doxorubicin and cyclophosphamide followed by docetaxel and trastuzumab (Tuma. J Natl Cancer Inst 2006 Mar. 1; 98(5):296-8). However, due to cardiac toxicity limitations, therapy may include shorter durations or lower doses (Tuma. J Natl Cancer Inst 2006 Mar. 1; 98(5):296-8). These types of anti-cancer therapy toxicities on normal cells could be avoided with a cancer cell-specific target such as Mig-7.

The data disclosed in the instant Example 1, indicate that anti-Mig-7 antibodies will be effective to inhibit invasion and metastasis in vivo. In addition, while agents such as Herceptin® (Trastuzumab) target only a fraction of breast cancers, Mig-7 is expressed in at least 96% (n=241) of all solid cancer types (Phillips and Lindsey. Oncology Reports 2005; 13:37-44). Therefore, according to particular aspects of the present invention, Mig-7 antibodies provide for highly selective, pan-cancer therapies, that not only provide for a broad therapeutic scope and efficacy, but also provide for decreased treatment costs. Moreover, Mig-7 antagonists (e.g., antibodies, siRNAs or antisense etc,) can be used alone, or in combination with current anti-growth factor therapies.

Targeting Mig-7 decreases chemoinvasion and enhances stimulation of human peripheral blood monocytes (MC) to produce cells that lyse MCF-7 tumor cells, and to produce TNF-α in vitro (see Example 1 herein). Enhanced killing indicates that Mig-7 is a tumor antigen target of stimulated human MC, and thus, provides proof that Mig-7 possesses tumor specific epitopes recognized by human MC. In addition, these experiments show that Mig-7 peptides combined with MUC1 are superior in stimulating MC to kill Mig-7 expressing carcinoma cells (e.g., MCF-7 cells). Furthermore, Mig-7 peptides, but not irrelevant peptides, enhance production of TNF-α, a MC cytokine known to cause tumor cell death (Bonta and Ben Efraim. Immunology Letters 1990; 25(4):295-301).

These results additionally indicate that inhibition of Mig-7 expression and/or function by antisense or by antibody treatments is suitable to inhibit carcinoma cell invasion in vivo.

In addition, Mig-7 peptides may serve to enhance immunotherapies. Recently it has been shown that vaccine to human papilloma virus strains that prevent cervical cancer are virtually 100% efficacious (Hildesheim and de Gonzalez. J Natl Cancer Inst 2006 Mar. 1; 98(5):292-3). According to additional aspects of the present invention, Mig-7-specific peptides provide novel vaccine compositions, and inoculating with such Mig-7-specific peptide-based vaccines provides novel methods for pan-cancer inoculation and treatment. In addition, Mig-7 peptides may be used ex vivo to stimulate immune cells that can then be infused into a patient for carcinoma cell killing in vivo.

Translation of the human carcinoma- and trophoblast-specific Mig-7 requires fidelity of the purine-pyrimidine repeat region and genetic recoding (see Example 2 herein). Applicant demonstrates, surprisingly, that a cancer-specific, “noncoding” mRNA produces protein when using Applicant's combination of techniques. Migration inducting gene-7 (Mig-7) is unique in its induction, translation and detection, making it highly cancer cell-specific. In Example 2, the Applicant details novel requirements for Mig-7 cloning and expression as well as relationship of these techniques to tumor progression. These techniques help elucidate the unique, tumor- and fetal/embryonic trophoblast-specific expression for use as a novel biomarker (e.g., diagnostic, and/or prognostic marker) and as a therapeutic target for cancer therapies.

Example 2 further shows that the polynucleotide sequence that encodes Mig-7 protein contains stop codons, but that Mig-7 mRNA contains predicted slippery and pseudoknot motifs known in other sequences to allow frameshifting and read-through of stop codons. Additionally, three conserved point mutations were detected in Mig-7: a G to C mutation at position 1048 of DQ080207; a C to T mutation at position 1112 of DQ080207; and a C to T mutation at position 1323 of DQ080207.

As described herein (Example 2), according to particular aspects of the present invention, protein/polypeptides of the invention not only encompass a polypeptide according to SEQ ID NO: 32 and contiguous portions thereof, but additionally encompasses polypeptides comprising the first nine amino acids MAASRCSGL (SEQ ID NO: 31), followed by one or more frame-shifted amino acid sequence selected from SEQ ID NOS: 32, 33 and 34.

In preferred aspects, after the ninth amino acid, the +1-reading frame (from MAASRCSGL in the 0-frame to SEMTLL . . . or to RSMTLL . . . or to MTLL in the +1-frame) is used, because this frameshift results in fewest downstream stops. This frame also contains a peptide that caused an increase breast carcinoma cell killing by peptide-stimulated monocytes from cancer patients in vitro. Moreover, overlapping peptides specific to this frame stimulate human peripheral blood monocyte killing of MCF-7 breast carcinoma cells in vitro in a statistically significant manner (see Example 1 herein). The TGA stop codon at position 532 can be read-through or frame shifted through, with the double-stop located between nucleotide positions 632 and 640 of SEQ ID NO: 35 being the likely ‘true stop’ given the 23 kD sized protein detected in immunoblots. However, because there are shift sites (slippery or “shifty” sites) at those stops as well, the protein may extend beyond the double-stop position shifting onto a different frame and end at a subsequent stop codon located between the double-stop and the canonical poly-A tail addition site (AATAAA) beginning at nucleotide position 757 of SEQ ID NO: 35. All such proteins are encompassed within the present invention.

Thus, particular aspects of the present invention provide a frame-shifted or genetically recoded Mig-7 polypeptide encoded by SEQ ID NO: 1, wherein said polypeptide comprises an amino-terminal MAASRCSGL amino acid sequence. In particular aspects, a Mig-7 polypeptide is from about 203 to about 207 amino acid residues in length. In particular aspects, the polypeptide is from about 223 to about 240 amino acid residues in length.

In certain aspects, a frame-shifted or genetically recoded Mig-7 polypeptide encoded by SEQ ID NO: 1, comprises an amino terminal MAASRCSGLSEMTLLGSQAVSGLSSPLKSPC amino acid sequence (SEQ ID NO: 51), an amino terminal MAASRCSGLRSEMTLLGSQAVSGLSSPLKSPC amino acid sequence (SEQ ID NO: 52), or an amino terminal MAASRCSGLLDSQKMTLLGSQAVSGLSSPLKSPC amino acid sequence (SEQ ID NO: 53). In particular aspects, a Mig-7 polypeptide is from about 203 to about 207 amino acid residues in length. In particular aspects, the polypeptide is from about 223 to about 240 amino acid residues in length.

In preferred aspects of the present invention, a Mig-7 polypeptide comprises an amino-terminal MAASRCSGL sequence linked to (e.g., via a peptide bond) at least one amino acid sequence selected from the group consisting of SEQ ID NOS: 32, 33, 34, and contiguous portions thereof. In particular aspects, a Mig-7 polypeptide is from about 203 to about 207 amino acid residues in length. In particular aspects, a Mig-7 polypeptide is from about 223 to about 240 amino acid residues in length.

Preferably, the amino-terminal MAASRCSGL sequence is linked to at least one amino acid sequence selected from the group consisting of SEQ ID NOS: 33, 34, and contiguous portions thereof. In particular aspects, a Mig-7 polypeptide is from about 203 to about 207 amino acid residues in length. In particular aspects, a Mig-7 polypeptide is from about 223 to about 240 amino acid residues in length.

Preferably, the amino-terminal MAASRCSGL sequence is linked to at least one amino acid sequence selected from the group consisting of SEQ ID NOS: 33, and contiguous portions thereof. In particular aspects, a Mig-7 polypeptide is from about 203 to about 207 amino acid residues in length. In particular aspects, a Mig-7 polypeptide is from about 223 to about 240 amino acid residues in length.

Expression of Mig-7 allows cancer cells to sense a 3-D environment, to invade and to form vessel structures (see Example 3 herein). Carcinoma cells and cytotrophoblasts both engage in vasculogenic mimicry. As described herein, Example 3 shows that Mig-7 is expressed by fetal/embryonic cytotrophoblasts and plays a role in their common cell behaviors of invasion and vessel formation. 3-D culture assays revealed that Mig-7 expression causes invasion and vessel formation. Adhesion assays to various extracellular matrix components suggests a mechanism for Mig-7 in tumor cell vessel formation is due to less adhesion to laminin. Example 3 further provides support for the efficacy of targeting Mig-7 in cancer therapies. The fact that Mig-7 is induced by growth factors that regulate PI3K, a signaling pathway required for vasculogenic mimicry, is localized to vessels in metastases, causes vessel-like formation and less adherence to laminin, collectively indicate that Mig-7 expression serves to allow cells to sense their environment, to invade and to cause vasculogenic mimicry. Therefore, according to particular aspects of the present invention, Mig-7 provides a molecular target for therapies to modulate tumor progression.

Mig-7 immunohistochemical data presented herein, further demonstrates Mig-7 specific expression in breast carcinoma tissue, and not to normal breast tissue. Although only a small core of the tumor tissue was taken for these analyses, the percentage of total breast carcinoma tissues staining positive for Mig-7 was 53%. One having ordinary skill in the art would recognize that the invasive front of carcinoma cells may not be well represented in small core samples. In fact, the present Applicant has previously demonstrated that 98% of cDNA from breast carcinoma tissue samples (n=50) contain moderate to high levels of Mig-7 (Phillips and Lindsey. Oncology Reports 2005; 13:37-44). Surprisingly, Mig-7 was identified in human breast adenosis with hyperplasia, which is considered “pre-cancerous” tissue. Similar Mig-7 positive pre-cancerous lesions were identified in endometrial tissues (Phillips and Lindsey. Oncology Reports 2005; 13:37-44). Thus, the present invention contemplates, in part, that Mig-7 can be targeted to inhibit cancer progression at early, as well as late, stage disease. In addition, Mig-7 is expressed in at least 87% (n=241) of all solid cancer types studied to date (Phillips and Lindsey. Oncology Reports 2005; 13:37-44), in contrast to HER2, which is limited in expression (Tuma. J Natl Cancer Inst 2006 Mar. 1; 98(5):296-8).

In various aspects, the present invention provides for methods to detect or diagnose breast cancer by detecting Mig-7 mRNA or protein expression in the breast tissue of a patient. Mig-7 mRNA or protein expression may be setected using any of the diagnostic methods presented herein or known in the art. The tissue sample may be collected from the breast, but may also be taken from the patient's blood, serum, sputum, urine, etc. For example, in one embodiment Mig-7 mRNA is detected in a patient using nucleic acid detection technology, including but not limited to PCR based methodologies. In another embodiment, Mig-7 antibodies are used to detect Mig-7 protein expression in a patient.

MT1-MMP activation causes MMP-2 (gelatinase A) activation (Lehti et al. Biochem J 1998; 334:345-53), increased invasion (Sato et al. Nature 1994; 370:61-5), and is required for tumor cell vasculogenic mimicry (Seftor et al. Cancer Res 2001; 61:6322-7). Thus, the present invention contemplates, in part, that Mig-7 expression aids in and/or facilitates MT1-MMP activation. Data presented herein (see Example 5) shows that inhibiting Mig-7 function and expression leads to decreased levels of active MT1-MMP; thus, providing a mechanism by which Mig-7 promotes invasion. MT1-MMP can be activated through the “cysteine switch” mechanism involving Mig-7 cysteine residues.

Analysis of protein phosphorylation in RL95 Mig-7 siRNA expressing cell lines in which levels of Mig-7 protein are decreased, demonstrated decreased levels of phosphorylated ERK1/2, Akt, and S6 kinase. Thus, the present invention contemplates, in part, that Mig-7 expression affects phosphorylation of these downstream signaling proteins. Analysis of IGF-1R and PRAS40 phosphorylation, showed no decrease due to a lack of Mig-7 protein, further supporting specific effects of Mig-7 expression on ERK1/2, Akt, and S6 kinase signaling molecules. IGF-1 receptor phosphorylation upregulates Mig-7 expression, but was not altered in cells with reduced Mig-7 protein levels. Thus, without wishing to be bound by any particular theory, the present invention contemplates, in part, that Mig-7 can act indirectly on the IGF-1 receptor through downstream targets. For example Mig-7 can act as an intermediate signaling protein downstream of RTK activation and αvβ5 integrin ligation and upstream of ERK 1/2, Akt, and S6 kinase. Activation of ERK1/2 and Akt downstream of RTK signaling can be caused by, as well as facilitate MT1-MMP activation (Lehti et al. Genes Dev 2005; 19:979-91; Takino et al. Cancer Res 2004; 64:1044-9; Tsubaki et al. Clinical and Experimental Metastasis 2007; 24:431-8). ERK1/2 and Akt phosphorylation signals invasion downstream of RTK activation and av integrin ligation (Hollier et al. Endocrinology 2008; 149:1075-90; Matsuo et al. Cancer Science 2006; 97:155-62; Zhou et al. Endocrinology 2007; 148:5195-208), which collaborate to induce endogenous Mig-7 (Crouch et al. Experimental Cell Research 2004; 292:274-87). Similarly, S6 kinase activation downstream of HGF, ERK1/2 and Akt signaling has been shown to be important for tumor cell migration (Wong et al. Experimental Cell Research 2004; 299:248-56). Thus, these findings establish a connection between the above-indicated signaling pathways and mechanisms of Mig-7 function and its effects on MT1-MMP activation.

Mig-7 expression leads to invasion and vessel-like structure formation in three dimensional cultures (Petty et al. Am J Pathol 2007; 170:1763-80). Thus, the present invention contemplates, in part, that inhibition of Mig-7 expression by stable Mig-7 specific siRNA expression can reduce early tumor growth in vivo. Significant decrease in tumor growth was observed days 13 and 15 after injection of carcinoma cells expressing Mig-7 specific siRNAs compared to control carcinoma cells (see Examples 4 and 5 herein). Reduction in tumor growth is accompanied by a reduction in Mig-7 expression; thus, inhibition of Mig-7 down-regulates vessel formation by aggressive tumor cells, i.e., vasculogenic mimicry. Based on these data and because Mig-7 facilitates invasion in vitro, reduction of Mig-7 expression in carcinoma cells can be used as a tool to slow primary tumor growth by inhibiting local tumor cell invasion.

Thus, the present invention contemplates, in part, to reduce tumor growth in vivo by administering a therapeutically effective amounts of a composition comprising at least one Mig-7 inhibitor to a patient. In particular aspects, the composition comprises one or more Mig-7 siRNAs. In other aspects, the composition comprises one or more Mig-7 antibodies in combination with one or more Mig-7 siRNAs.

Vasculogenic mimicry requires aggressive invasion (Maniotis et al. Am J Pathol 1999; 155:739-52) and both correlate well with poor prognosis and poor outcome (Sood et al. Am J Pathol 2001; 158:1279-88; Folberg et al. Am J Pathol 2000; 156:361-81). Furthermore, when cancer cells are invading or circulating they are resistant to apoptosis and current therapies; thus, targeting carcinoma specific genes that are involved in invasion, can lead to more effective treatments with less recurrence due to residual invading or circulating cancer cells that were not killed by standard therapies. Therefore, inhibition of Mig-7 expression or function is a suitable therapeutic strategy to inhibit carcinoma cell invasion and disease progression in vivo.

### Peptide-Based Vaccines:

Peptide-based vaccines are well known in the art, and may contain additional antigenic and adjuvant elements. Peptide-based vaccines are advantageous over traditional vaccines for several reasons: they are substantially safer; they have a relatively long shelf-life; they have the ability to target the immune response towards specific epitopes that are not suppressive or hazardous for the host; and they offer the possibility of preparing multi-component and multi-pathogen vaccines.

The efficacy of inventive peptide-based vaccines is enhanced by adequate presentation of the epitopes to the immune system. Therefore, in preferred aspects, the Mig-7 based polypeptides or epitopes are coupled to, or are expressed (e.g, hybrid-gene expression) as part of, a carrier that may also offer an adjuvant function. Additional adjuvants may or may not be included in the immunization.

In particular aspects, immunizations are performed with one or more Mig-7 polypeptides as disclosed herein.

### Antibodies

In particular aspects, Mig-7 polypeptides have utility for developing respective antibodies (e.g., monoclonal antibodies), and compositions comprising such antibodies.

Such antibodies and compositions have utility as novel diagnostic reagents for directly detecting the respective condition (e.g., cancer). The diagnostic assays are rapid, high-throughput and suitable for ‘point-of-care’ implementations.

Diagnostic Assays

Particular aspects of the present invention thus provide a high-throughput method for detecting cancer, comprising: obtaining a test sample from a test subject; and detecting Mig-7 in the sample using an immunologic assay based, at least in part, by using at least one antibody reagent, or epitope-binding portion thereof, specific for a Mig-7 protein or polypeptide antigen as disclosed herein.

In particular aspects, the immunologic assay is selected from the group consisting of ELISA, immunoprecipitation, immunocytochemistry, immunoelectrophoresis, immunochemical methods, Western analysis, antigen-capture assays, two-antibody sandwich assays, binder-ligand assays, agglutination assays, complement assays, and combinations thereof. In particular aspects, the antibody is selected from the group consisting of: a single-chain antibody, a monoclonal antibody, a chimeric antibody, a humanized antibody, and a Fab fragment. In particular aspects, a plurality of antibodies, or eptitope-binding portions thereof, are used, in each case specific for a Mig-7 protein or polypeptide antigen as disclosed herein.

Therapeutic Agents

Additionally, because of the nature of the relevant specific binding interactions, antibodies and antibody-containing compositions of the present invention have therapeutic utility for treatment or prevention of cancer. The inventive antibodies and antibody compositions have utility for treating cancer, for alleviating symptoms of cancer, and/or to prevent cancer. Preferably, the antibodies and antibody compositions are directed against Mig-7 epitopes, and can be used to treat or prevent cancer by administration to subjects in need thereof.

Specifically, particular aspects of the present invention provide an antibody directed against a Mig-7 protein or polypeptide antigen as disclosed herein.

In particular aspects, the antibody is a monoclonal antibody, or antigen-binding portion thereof. In particular aspects, the monoclonal antibody, or antigen-binding portion thereof, is a single-chain antibody, chimeric antibody, humanized antibody or Fab fragment.

Additional aspects provide a composition, comprising at least one of the above-described antibodies. Preferably, the composition comprises a Mig-7-specific monoclonal antibody. Preferably, at least one of the antibodies forms specific immunocomplexes with Mig-7 proteins or polypeptides associated with cancer cells.

Yet further aspects provide a pharmaceutical composition, comprising at least one of the above-described antibodies, along with a pharmaceutically acceptable diluent, carrier or excipient. Preferably, the composition is administered to a subject, whereby the composition prevents or inhibits cancer. In particular aspects, the composition is administered to a subject, whereby the composition ameliorates symptoms of cancer. In particular aspects, at least one of the antibodies of the composition forms specific immunocomplexes with Mig-7 proteins or polypeptides associated with cancer cells.

Yet further aspects provide a method of treating, or of preventing cancer, comprising administering to a subject in need thereof, a therapeutically effective amount of at least one of the above-described antibodies, or of a pharmaceutical composition comprising at least one of the antibodies. In particular aspects, the immunoglobulin sequences are, or substantially are, human immunoglobulin sequences.

Arrays

Yet further aspects provide an array of different Mig-7 proteins or polypeptide epitopes (oligopeptides) immobilized on a solid phase. The term “microarray” refers broadly to both ‘polypeptide microarrays’ and ‘polypeptide chip(s),’ and encompasses all art-recognized solid supports, and all art-recognized methods for synthesizing polypeptides on, or affixing polypeptides molecules thereto. The solid-phase surface may comprise, from among a variety of art-recognized materials, silicon, glass, polystyrene, aluminum, steel, iron, copper, nickel, silver, gold or cellulose. However, nitrocellulose as well as plastics such as nylon, which can exist in the form of pellets (e.g., beads) or also as resin matrices, may also be used.

The present invention contemplates, in part, that Mig-7 oligopeptides, or particular sequences thereof, may constitute all or part of an “virtual array” wherein the oligopeptides, or particular sequences thereof, are used, for example, as ‘specifiers’ as part of, or in combination with a diverse population of unique labeled oligopeptides to analyze a complex mixture of analytes. In such methods, enough labels are generated so that each antibody in the complex mixture (i.e., each analyte) can be uniquely bound by a unique label, and thus, can be detected (e.g., each label may be directly counted, resulting in a digital read-out of each molecular species in the mixture).

Preferred aspects of the present invention provide an array comprising a plurality of different Mig-7 proteins or polypeptides coupled to a solid phase.

Preferably, the solid phase comprises a material selected from the group consisting of silicon, cellulose, glass, polystyrene, polyacrylamide, aluminum, steel, iron, copper, nickel, silver, gold and combinations thereof.

### Immunologic Assays

According to the present invention, numerous art-recognized competitive and non-competitive protein binding immunoassays are used to detect and/or quantify antigens or antibodies (e.g., Harlow & Lane, Antibodies: A Laboratory Manual; Cold Spring Harbor Laboratory, New York 555-612, 1988). Such immunoassays can be qualitative or and/or quantitative, and include, but are not limited to antibody capture assays, antigen capture assays, and two-antibody sandwich assays, immunodiffusion, immunoelectrophoresis, immunochemical methods, binder-ligand assays, immunohistochemical techniques, agglutination and complement assays (e.g., Basic and Clinical Immunology, 217-262, Sites and Terr, eds., Appleton & Lange, Norwalk, Conn., 1991 which is incorporated herein by reference). Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Antibody capture assays comprise immobilizing an antigen on a solid support, and contacting the immobilized antigen with an antibody-containing solution, whereby antigen-specific antibody, if present, binds to the immobilized antigen. The antibodies can be labeled or unlabeled. Antigen attachment to the solid support is typically non-covalent, but might in particular instances be covalent. After washing the support, antibody retained on the solid support is detected, or quantified by measuring the amount thereof. ELISA assays represent preferred aspects of immunologic antibody capture assays as used herein. Competitive ELISA assays represent a preferred aspect of antibody capture assay, wherein the antigen is bound to the solid support and two antibodies which bind the antigen are allowed to compete for binding of the antigen. The amount of monoclonal antibody bound is measured, and a determination made as to whether the serum contains the cognate antigen antibodies. Such ELISAs can be used to indicate immunity to known protective epitopes in a vaccinee following vaccination.

Antigen capture assays comprise immobilizing an antibody to a solid support, and contacting the immobilized antibody with an antigen-containing solution, whereby antibody-specific antigen, if present, binds to the immobilized antibody. The antigens can be labeled or unlabeled. Antibody attachment to the solid support is typically non-covalent, but might in particular instances be covalent. After washing the support, antigen retained on the solid support is detected, or quantified by measuring the amount thereof.

Two-antibody sandwich assays (e.g., in the context of an antigen-capture assay) comprise initially immobilizing a first antigen-specific antibody on a solid support, followed by contacting the immobilized antibody with antigen-containing solution, washing the support, and subsequently detecting or quantifying the amount of bound antigen by contacting the immobilized antibody-antigen complexes with a second antigen-specific antibody, and measuring the amount of bound second antibody after washing.

Generally, immunoassays rely on labeled antigens, antibodies, or secondary reagents for detection. These proteins (antigens or antibodies) can be labeled with radioactive compounds, enzymes (e.g. peroxidase), biotin, or fluorochromes, etc. Enzyme-conjugated labels are particularly useful when radioactivity must be avoided, and provides for relatively rapid results. Biotin-coupled reagents are typically detected with labeled streptavidin. Streptavidin binds tightly and quickly to biotin and can be labeled with radioisotopes or enzymes. Fluorochromes provide a very sensitive method of detection. Antibodies useful in these assays include, but are not limited to, monoclonal antibodies, polyclonal antibodies, affinity-purified polyclonal antibodies, and antigen or epitope-binding fragments of any of these. Labeling of antibodies or fragments thereof can be accomplished using a variety of art-recognized techniques (e.g., Kennedy et al., Clin. Chim. Acta., 70:1-31, 1976; Schurs et al., Clin. Chim Acta., 81:1-40, 1977; both incorporated by reference herein). Coupling techniques include, but are not limited to, the glutaraldehyde periodate method, dimaleimide, and other methods

ELISA

Enzyme-linked immunosorbent assay (ELISA) systems are widely recognized in the art, and are commonly used to detect antibodies in, for example, serum samples. For detection of antibodies in serum, a serum sample, or diluted serum sample, is applied to a surface (e.g. a well of a microtiter plate, preferably ‘blocked’ to reduce non-specific protein binding) having immobilized antigens (epitope(s)) thereon. Serum antibodies specific for the immobilized epitope(s) bind with high affinity to the immobilized epitope(s) on the plate, and are retained after standard washes, whereas non-specific antibodies do not bind with high affinity, and are removed after standard washes.

Specifically bound antibody is detected, for example, by using enzyme-coupled anti-immunoglobulins and a chromogen (e.g., horseradish peroxidase-conjugated antibodies used in combination with hydrogen peroxide). The enzyme which is bound to the antibody will react with an appropriate substrate, preferably a chromogenic substrate, in such a manner as to produce a chemical moiety which can be detected, for example, by spectrophotometric, fluorimetirc or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

The detection can be accomplished by calorimetric methods that employ a chromogenic substrate for the enzyme. Detection may also be accomplished visually by comparison of the extent of enzymatic reaction with appropriate standards. Detection may also be accomplished using any of a variety of other immunoassays. For example, by radioactively labeling the antibodies or antibody fragments, it is possible to detect antigenic peptides through the use of a radioimmunoassay (RIA). The radioactive isotope can be detected by such means as the use of a gamma counter or a scintillation counter or by autoradiography. It is also possible to label the antibody with a fluorescent compound. When the fluorescently labeled antibody is exposed to light of the proper wavelength, its presence can be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### Generation and Production of Antibodies

Polyclonal or monoclonal antibodies to Mig-7 proteins and polypeptides or to epitope-bearing fragments thereof can be made for therapeutic, or diagnostic (e.g., immunoassays) use by any of a number of methods known in the art. By epitope, reference is made to an antigenic determinant of a polypeptide. An epitope could comprise 3 amino acids in a spatial conformation which is unique to the epitope (methods of determining the spatial conformation of amino acids are known in the art, and include, for example, x-ray crystallography and 2 dimensional nuclear magnetic resonance). Generally an epitope consists of at least 5 such amino acids. The present invention encompasses epitopes and/or polypeptides recognized by antibodies of the present invention, along with conservative substitutions thereof, which are still recognized by the antibodies.

One approach for preparing antibodies to a protein is the selection and preparation of an amino acid sequence of all or part of the protein, chemically synthesizing the sequence and injecting it into an appropriate animal, usually a rabbit or a mouse.

Oligopeptides can be selected as candidates for the production of an antibody to Mig-7 proteins or polypeptides based upon the oligopeptides lying in hydrophilic regions, which are thus likely to be exposed in the mature protein.

Alternatively, proteins and polypeptides can be selected by other art-recognized methods. Additionally, a combination of selection methods can be used.

Preferred proteins and polypeptides of the present invention are those of Mig-7, proteins and polypeptides and epitope-bearing fragments thereof.

In particular aspects of the present invention, a Mig-7 protein or polypeptide comprises at least one epitope of a sequence disclosed herein.

Methods for preparation of Mig-7 proteins or polypeptides, or of an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E.I. du Pont de Nemours Company, Wilmington, Del.) (Caprino and Han, J Org Chem 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified Mig-7 proteins or polypeptides usually by ELISA or by bioassay based upon the ability to block the action of Mig-7 proteins or polypeptides. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating cancer and related disease. Thus, another aspect of the present invention provides for a method for preventing or treating cancer and related diseases involving treatment of a subject with specific antibodies to Mig-7 proteins or polypeptides.

Specific antibodies, either polyclonal or monoclonal, to Mig-7 proteins or polypeptides can be produced by any suitable method known in the art as discussed above. For example, murine or human monoclonal antibodies can be produced by hybridoma technology or, alternatively, Mig-7 proteins or polypeptides, or an immunologically active fragment thereof, or an anti-idiotypic antibody, or fragment thereof can be administered to an animal to elicit the production of antibodies capable of recognizing and binding to Mig-7 proteins or polypeptides. Such antibodies can be from any class of antibodies including, but not limited to IgG, IgA, IgM, IgD, and IgE or in the case of avian species, IgY and from any subclass of antibodies.

The present invention further provides for methods to detect the presence of Mig-7 proteins or polypeptides in a sample obtained from a patient. As discussed above under “Immunologic Assays,” any method known in the art for detecting proteins can be used. Such methods include, but are not limited to immunodiffusion, immunoelectrophoresis, immunochemical methods, binder-ligand assays, immunohistochemical techniques, agglutination and complement assays. (for example, see Basic and Clinical Immunology, 217-262, Sites and Terr, eds., Appleton & Lange, Norwalk, Conn., 1991 which is incorporated by reference). Preferred are ELISA methods, including reacting antibodies with an epitope or epitopes of Mig-7 proteins or polypeptides.

As provided herein, the compositions and methods for diagnosis/detection of cancer, or the therapeutic methods of treatment or prevention provided herein, may utilize one or more antibodies used singularly, or in combination with other therapeutics to achieve the desired effects. Antibodies according to the present invention may be isolated from an animal producing the antibody as a result of either direct contact with an environmental antigen or immunization with the antigen. Alternatively, antibodies may be produced by recombinant DNA methodology using one of the antibody expression systems well known in the art (see, e.g., Harlow & Lane, Antibodies: A Laboratory Manual, Cold Spring Harbor Laboratory (1988). Such antibodies may include recombinant IgGs, chimeric fusion proteins having immunoglobulin derived sequences or “humanized” antibodies that may all be used according to the present inventive aspects. In addition to intact, full-length molecules, the term antibody also refers to fragments thereof (e.g., scFv, Fv, Fd, Fab, Fab′ and F(ab)′₂ fragments), or multimers or aggregates of intact molecules and/or fragments that bind to the inventive antigens (proteins/polypeptides/epitopes). These antibody fragments bind antigen and may be derivatized to exhibit structural features that facilitate clearance and uptake (e.g., by incorporation of galactose residues).

In particular aspects antibodies are monoclonal antibodies, prepared essentially as described in Halenbeck et al. U.S. Pat. No. 5,491,065 (1997), incorporated herein by reference.

Additional aspects comprise humanized monoclonal antibodies. The phrase “humanized antibody” refers to an antibody initially derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are less immunogenic in humans than the parental mouse monoclonal antibodies, they can be used for the treatment of humans with far less risk of anaphylaxis. Thus, these antibodies may be preferred in therapeutic applications that involve in vivo administration to a human.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, for example, Jones et al., Nature 321:522-525, 1986; Morrison et al., Proc. Natl. Acad. Sci., U.S.A., 81:6851-6855, 1984; Morrison and Oi, Adv. Immunol., 44:65-92, 1988; Verhoeyer et al., Science 239:1534-1536, 1988; Padlan, Molec. Immun. 28:489-498, 1991; Padlan, Molec. Immunol. 31(3):169-217, 1994; and Kettleborough, C. A. et al., Protein Eng. 4(7):773-83, 1991, each of which is incorporated herein by reference.

The phrase “complementarity determining region” refers to amino acid sequences which together define the binding affinity and specificity of the natural Fv region of a native immunoglobulin binding site (see, e.g., Chothia et al., J. Mol. Biol. 196:901-917, 1987; Kabat et al., U.S. Dept. of Health and Human Services NIH Publication No. 91-3242, 1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance (e.g., via Ashwell receptors) (see, e.g., U.S. Pat. Nos. 5,530,101 and 5,585,089, both incorporated herein by reference).

Humanized antibodies to the inventive proteins can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/00741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735; disclosing monoclonal antibodies against a variety of antigenic molecules including IL-6, IL-8, TNFa, human CD4, L-selectin, gp39, and tetanus toxin. The monoclonal antibodies can be tested for the ability to inhibit or neutralize the biological activity or physiological effect of the corresponding protein or pathogenic agent (e.g., virus). WO 96/3373 discloses that monoclonal antibodies against IL-8, derived from immune cells of transgenic mice immunized with IL-8, blocked IL-8 induced functions of neutrophils. Human monoclonal antibodies with specificity for the antigen used to immunize transgenic animals are also disclosed in WO 96/34096. The antibodies of the present invention are said to be immunospecific, or specifically binding, if they bind to the Mig-7 antigen (protein/polypeptide/epitope) with a Kₐ of greater than or equal to about 10⁴ M⁻¹, preferably of greater than or equal to about 10⁶ M⁻¹, more preferably of greater than or equal to about 10⁶ M⁻¹, and still more preferably of greater than or equal to about 10⁷ M⁻¹. Such affinities may be readily determined using conventional techniques, such as by equilibrium dialysis; by using the BIAcore 2000 instrument, using general procedures outlined by the manufacturer; by radioimmunoassay using ¹²⁵I-labeled proteins; or by another method known to the skilled artisan. The affinity data may be analyzed, for example, by the method of Scatchard et al., Ann N.Y. Acad. Sci., 51:660, 1949. Thus, it will be apparent that preferred antibodies will exhibit a high degree of specificity for the Mig-7 antigen of interest, and will bind with substantially lower affinity to other molecules.

Preferably the anti-pathogenic antibodies of the present invention are monoclonal antibodies. More preferably, the antibodies are humanized monoclonal antibodies.

### Biologically Active Variants

Variants of Mig-7 polypeptides have substantial utility in various aspects of the present invention. Variants can be naturally or non-naturally occurring. Naturally occurring variants are found in humans or other species and comprise amino acid sequences which are substantially identical to the amino acid sequences shown herein, and include natural sequence polymorphisms. Species homologs of the protein can be obtained using subgenomic polynucleotides of the invention, as described below, to make suitable probes or primers for screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants, including the Mig-7 activities disclosed herein and the modulation of target signaling activity, are also included here. Preferably, naturally or non-naturally occurring variants have amino acid sequences which are at least 85%, 90%, or 95% identical to the amino acid sequence shown herein. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R., §§1.821-1.822, abbreviations for amino acid residues are shown in Table 1:

TABLE 1
Table of Amino Acid Nomenclature

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

Such substitutions may be made in accordance with those set forth in TABLE 2 as follows:

TABLE 2
Conservative Amino Acid Substitutions

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

Variants of the Mig-7 polypeptide disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (Mark et al., U.S. Pat. No. 4,959,314).

Preferably, amino acid changes in the Mig-7 polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of Mig-7 polypeptides or polypeptide variants are of the same type as a protein comprising the amino acid sequence encoded by the nucleotide sequences shown herein, although the properties and functions of variants can differ in degree.

Mig-7 polypeptide variants include glycosylated forms, aggregative conjugates with other molecules, and covalent conjugates with unrelated chemical moieties (e.g., pegylated molecules). Mig-7 polypeptide variants also include allelic variants (e.g., polymorphisms), species variants, and muteins. Truncations or deletions of regions which do not preclude functional activity of the proteins are also variants. Covalent variants can be prepared by linking functionalities to groups which are found in the amino acid chain or at the N- or C-terminal residue, as is known in the art.

It will be recognized in the art that some amino acid sequence of Mig-7 polypeptides of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors (Ostade et al., Nature 361:266-268, 1993). Thus, Mig-7 polypeptides of the present invention may include one or more amino acid substitutions, deletions or additions, either from natural mutations or human manipulation.

Of particular interest are substitutions of charged amino acids with another charged amino acid and with neutral or negatively charged amino acids. The latter results in proteins with reduced positive charge to improve the characteristics of the disclosed proteins. The prevention of aggregation is highly desirable. Aggregation of proteins not only results in a loss of activity but can also be problematic when preparing pharmaceutical formulations, because they can be immunogenic (Pinckard et al., Clin. Exp. Immunol. 2:331-340, 1967; Robbins et al., Diabetes 36:838-845, 1987; Cleland et al., Crit. Rev. Therapeutic Drug Carrier Systems 10:307-377, 1993).

Amino acids in Mig-7 polypeptides of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904, 1992 and de Vos et al. Science 255:306-312, 1992).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given Mig-7 polypeptide will not be more than 50, 40, 30, 25, 20, 15, 10, 5 or 3.

In addition, pegylation of Mig-7 polypeptides and/or muteins is expected to provide improved properties, such as increased half-life, solubility, and protease resistance. Pegylation is well known in the art.

### Fusion Proteins

Fusion proteins comprising proteins or polypeptide fragments of Mig-7 polypeptides can also be constructed. Fusion proteins are useful for generating antibodies against amino acid sequences and for use in various targeting and assay systems. For example, fusion proteins can be used to identify proteins which interact with a Mig-7 polypeptide of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence can be used.

A fusion protein comprises two protein segments fused together by means of a peptide bond. Amino acid sequences for use in fusion proteins of the invention can be utilize the amino acid sequence shown herein (e.g., contiguous amino acid residues corresponding to different reading frames of the Mig-7 coding sequence; frame-shift protein variants, etc) or can be prepared from biologically active variants thereof. The first protein segment can include a full-length Mig-7 polypeptide.

Other first protein segments can consist of amino acid sequence selected from the group consisting of SEQ ID NOS: 38, 39, 40, 41, 42, fragments of SEQ ID NOS: 38 and 39 of about 17 to 85 contiguous residues in length, and fragments of SEQ ID NOS: 40, 41 and 42 of about 8 to 81 contiguous residues in length, and combinations thereof, wherein the polypeptide optionally comprises an amino terminal MAASRCSGL sequence.

The second protein segment can be a full-length protein or a polypeptide fragment. The second protein can be homologous or heterologous. Heterologous proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP16 protein fusions.

These fusions can be made, for example, by the frame-shift process described herein for Mig-7 expression, or alternatively by covalently linking two protein segments or by standard procedures in the art of molecular biology. Recombinant DNA methods can be used to prepare fusion proteins, for example, by making a DNA construct which comprises a coding region for the Mig-7 protein sequence in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### RNA Interference

RNA interference (RNAi) are gene regulatory RNAs that can be used for studying the effects of inhibiting gene expression. RNAi can occur by way of small interfering RNA (also called short interfering RNA or silencing) (sRNA), or microRNA (miRNA). (See, for example, U.S. Pat. No. 6,506,559; Milhavet et al., Pharm. Rev. 55:629-648, 2003; and Gitlin et al., J. Virol. 77:7159-7165, 2003; incorporated herein by reference).

The phrase “contacting a cell,” and any derivations thereof as used herein, refers to methods of exposing a cell, delivering to a cell, or ‘loading’ a cell with an agent (e.g., sRNA agents, antisense agents, ribozyme agents, antibodies, etc) whether directly or indirectly by viral or non-viral vectors, and wherein such agent is bioactive upon delivery. The method of delivery will be chosen for the particular agent and use (e.g., cancer being treated). Parameters that affect delivery, as is known in the medical art, can include, inter alia, the cell type affected (e.g. tumor), and cellular location.

Inhibition of gene expression refers to the absence (or observable decrease) in the level of protein and/or mRNA product from a gene target. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS), inhibition of Mig-7-induced proliferation, or inhibition of Mig-7 induced cellular phenotype, including vascular mimicry as described herein. For RNA-mediated inhibition in a cell line or whole organism, gene expression is conveniently assayed by use of a reporter or drug resistance gene whose protein product is easily assayed. Many such reporter genes are known in the art.

The invention, in particular aspects, contemplates introduction of RNA with partial or fully double-stranded character into the cell or into the extracellular environment. According to the present invention, inhibition is specific to the particular Mig-7 cellular gene expression product in that a nucleotide sequence from a portion of the sequence is chosen to produce inhibitory RNA. This process is effective in producing inhibition (partial or complete), and is gene-specific. In particular aspects, the target cell containing the sRNA or miRNA may be a mammalian cell, in vitro or in vivo. Methods of preparing and using sRNA or miRNA are generally disclosed in U.S. Pat. No. 6,506,559, incorporated herein by reference (see also reviews by Milhavet et al., Pharmacological Reviews 55:629-648, 2003; and Gitlin et al., J. Virol. 77:7159-7165, 2003; incorporated herein by reference).

The sRNA may further comprise one or more strands of polymerized ribonucleotide, and may include modifications to either the phosphate-sugar backbone or the nucleoside, and may contain non-natural amino acids (e.g. amino acid analogs). The phosphodiester linkages of natural RNA may be modified to include at least one of a nitrogen or sulfur heteroatom. Modifications in RNA structure may be tailored to allow specific genetic inhibition while avoiding a general immune-based response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. Nucleic acid containing a nucleotide sequence identical to a portion of the validated gene sequence is preferred for inhibition. RNA sequences with insertions, deletions, and single point mutations relative to the target sequence may also be effective for inhibition. Sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript. RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For siRNA (RNAi or short hairpin RNA), the RNA may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

RNA containing a nucleotide sequences identical to a portion of a particular gene sequence are preferred for inhibition. RNA sequences with insertions, deletions, and single point mutations relative to the target sequence may be effective for inhibition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of particular gene sequence is preferred. Alternatively, the duplex region of the RNA may be defined functionally as a nucleotide sequence that is capable of hybridizing with a portion of the particular gene transcript (e.g., 400 mM NaCl, 40 mM PIPES pH 6.4, 1 mM EDTA, 50° C. or 70° C. hybridization for 12-16 hours; followed by washing).

Preferably, wherein the siRNA agent specific for a Mig-7-induced cellular gene sequence comprises a nucleic acid sequence of, e.g., at least 9, at least 15, at least 18, or at least 20 contiguous bases in length that is complementary to, or hybridizes under moderately stringent or stringent conditions to a sequence selected from the group consisting of SEQ ID NOs: 43-49, and sequences complementary thereto.

In another aspect, the siRNA agent specific for a Mig-7-induced cellular gene sequence comprises a nucleic acid sequence of, e.g., at least 9, at least 15, at least 18, or at least 20 contiguous bases in length that is complementary to, or hybridizes under moderately stringent or stringent conditions to a sequence a Mig-7 nucleic acid sequence as disclosed herein (e.g., SEQ ID NO: 1), and sequences complementary thereto. A 100% sequence identity between the RNA and a particular gene sequence is not required to practice the present invention.

Furthermore, it would be understood by one having ordinary skill in the art that an siRNA construct comprises bases A, C, T, and G or variants thereof; whereas an siRNA comprises the transcribed product of the siRNA construct (e.g., bases A, C, T, and U). The skilled artisan would also appreciate that the present invention also includes siRNAs synthesized directly, i.e., without being transcribed from an siRNA construct. Exemplary siRNAs are set forth in SEQ ID NOs: 54-60.

Thus, the methods have the advantage of being able to tolerate sequence variations that might be expected due to genetic mutation, strain polymorphism, or evolutionary divergence. Sequences with greater than about 90% identity, greater than about 91% identity, greater than about 92% identity, greater than about 93% identity, greater than about 94% identity, greater than about 95% identity, greater than about 96% identity, greater than about 97% identity, greater than about 98% identity, greater than about 99% identity, greater than about 99.5% identity, greater than about 99.9% identity, or any value therebetween may also be used with the present invention.

Particular gene sequence siRNA may be synthesized by art-recognized methods either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (e.g., WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

The siRNA may be used alone or as a component of a kit having at least one of the reagents necessary to carry out the in vitro or in vivo introduction of RNA to test samples or subjects. Preferred components are the dsRNA and a vehicle that promotes introduction of the dsRNA. Such a kit may also include instructions to allow a user of the kit to practice the invention.

Further, one or more siRNA of the present invention may comprise a pharmaceutical or therapeutic composition that may be useful, for example, as an anti-cancer agent. Such pharmaceutical or therapeutic composition may further comprise inert ingredients, such as carriers described herein at other sections.

### Cell Targeting

According to additional preferred aspects of the present invention, anti-Mig-7 antibodies can be used to target Mig-7 on cells (e.g., cancer cells, or fetal cytotrophoblast cells). Anti-Mig-7 antibody-based agents can be used to deliver a locally acting biological agent that will affect the targeted cell.

Mig-7 in the context of the inventive targeting, is expressed on the surface of cancer cells and is accessible. For example, where Mig-7 is present at higher levels on particular Mig-7-bearing cells (e.g., cancer cells, or fetal cytotrophoblast cells) as compared to other cells, they can be utilized as preferential targets for systemic anti-Mig-7 antibody-based agents and therapies. Mig-7 differential expression enables the specific targeting of anti-Mig-7 antibody-based agents. Anti-Mig-7 antibody-based agents (e.g., drugs, cytoxic agents, labeling agents, etc.) directed against the Mig-7 expressing cells (i.e., targets) preferentially affect the targeted cell over normal tissue. For example, an anti-Mig-7 antibody-drug conjugate that predominantly binds Mig-7 on particular cells (e.g., cancer cells, or fetal cytotrophoblast cells) would be expected to selectively affect those cells within a treated individual. Preferably, Mig-7 is accessible to the anti-Mig-7 antibody-based agent, and is found in substantially greater concentrations on the targeted cells relative to other cells that lack Mig-7 expression or that express Mig-7 at relatively low levels.

Therefore, the present invention includes, but is not limited to, anti-Mig-7 antibody-based agents specific to Mig-7 expressing target cells that will enable or facilitate therapeutic treatments relating to, for example, cancer cells, or fetal cytotrophoblast cells.

In particular aspects, Herstatin- and/or RBD Int8 polypeptides are conjugated or coupled to drugs, or to toxins.

In alternate aspects, anti-Mig-7 antibodies, or antigen-binding fragments thereof are conjugated or coupled to radionuclides.

Additional aspects provide for anti-Mig-7 antibody-coated liposomes that contain one or more biologically active compounds.

In preferred aspects, anti-Mig-7 antibody-mediated targeting is used to deliver drugs or other agents to cancer cells, or fetal cytotrophoblast cells.

In alternative aspects of the present invention, targeted binding of an anti-Mig-7 antibody-based agent to a cell is sufficient to modulate Mig-7-mediated cell signaling cascades, inhibit or alter cell growth (e.g., cytostatic effects) or even kill the target cell (cytotoxic effects) if desired. The mechanisms of these activities can vary, but involve Mig-7 polypeptide-dependent receptor activation, changes in receptor expression, cell-mediated cytotoxicity, activation of apoptosis, inhibition of ligand-receptor function, or provide a signal for complement fixation. In fact, an anti-Mig-7 antibody-based agent may exhibit one or several such activities. In particular aspects, anti-Mig-7 antibody-based agents are cytostatic, but not cytotoxic. In particular related aspects, anti-Mig-7 antibody-based agents bind to Mig-7 expressing target cells, and modulate cell signaling cascades and cellular metabolism, or are either cytoxic or cytostatic, etc.

In additional aspects, anti-Mig-7 antibody-based agent are conjugated or coupled to a diverse array of compounds which include, but are not limited to, proteins, drugs, toxins or cytotoxic agents, cytostatic agents, radionuclides, apoptotic factors (Wuest et al. 2002), anti-angiogenic compounds or other biologically active compounds that affect cellular signaling or metabolism, inhibit cell growth or even kill a target cell or tissue. For example, cytotoxic or cytostatic agents include, but are not limited to, diphtheria toxin and Pseudomonas exotoxin (Kreitman 2001 a; Kreitman 2001 b), ricin (Kreitman 2001a), gelonin, doxorubicin (Ajani et al. 2000) and its derivatives, iodine-131, yttrium-90 (Witzig 2001), indium-111 (Witzig 2001), RNase (Newton and Ryback 2001), calicheamicin (Bernstein 2000), apoptotic agents, and antiangiogenic agents (Frankel et al. 2000; Brinkmann et al. 2001; Garnett 2001). According to particular aspects of the present invention, anti-Mig-7 antibodies coupled to these compounds are used to adversely affect cells displaying Mig-7.

Toxins can also be targeted to specific cells by incorporation of the toxin into anti-Mig-7 antibody-coated liposomes. Anti-Mig-7 antibody-based agents direct liposomes to a Mig-7 expressing target cell where the bioactive compound is released. For example, cytotoxins in anti-Mig-7 antibody-coated liposomes are used to treat cancer. In alternate aspects, these targeted liposomes are loaded with DNA encoding bioactive polypeptides (e.g., inducible nitric oxide synthase; Khare et al. 2001).

Prodrugs or enzymes can also be delivered to targeted cells by specific anti-Mig-7 antibody-based agents. In this case the conjugate consists of an anti-Mig-7 antibody coupled to a drug that can be activated once the polypeptide agent binds the target cell. Examples of this strategy using antibodies have been reviewed (Denny 2001; Xu and McLeod 2001).

Therefore, in particular aspects, anti-Mig-7 antibody-prodrug/enzyme conjugates that are targeted to one or more Mig-7 target cells have utility for the treatment of, for example, cancer and other treatable conditions discussed herein.

The specificity and high affinity of anti-Mig-7 antibody-based agents makes them ideal candidates for delivery of toxic agents to a specific subset of cellular targets. Preferably, one or more Mig-7 targets are exclusively present, or present at higher levels on the target cells (e.g., cancer, tumor cells) than on non-cancer cells.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

In particular aspects, a therapeutic effect may also encompass prophylaxis of symptoms of a condition.

As used herein, the term “subject” refers to animals, including mammals, such as human beings. As used herein, a patient refers to a human subject.

As used herein, the phrase “associated with” or “characterized by” refers to certain biological aspects such as expression of a receptor or signaling by a receptor that occurs in the context of a disease or condition. Such biological aspects may or may not be causative or integral to the disease or condition but merely an aspect of the disease or condition.

As used herein, a biological activity refers to a function of a polypeptide including but not limited to complexation, dimerization, multimerization, receptor-associated kinase activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. A biological activity can be assessed by assays described herein and by any suitable assays known to those of skill in the art, including, but not limited to in vitro assays, including cell-based assays, in vivo assays, including assays in animal models for particular diseases.

### Pharmaceutical Compositions and Therapeutic Uses

Pharmaceutical compositions of particular aspects of the invention comprise one or more Mig-7 polypeptides, or anti-Mig-7 antibody-based agents of the claimed invention in a therapeutically effective amount. The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of Mig-7 polypeptides, or anti-Mig-7 antibody-based agents in the individual to which it is administered. A non-limiting example of a pharmaceutical composition is a composition that either enhances or diminishes signaling mediated by the Mig-7 target. Where such signaling modulates a disease-related process, modulation of the signaling would be the goal of the therapy.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., New Jersey, 1991).

### Delivery Methods

Once formulated, the compositions of the invention can be administered (as proteins/polypeptides, or in the context of expression vectors for gene therapy) directly to the subject or delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, myocardial, intratumoral, peritumoral, or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in, for example, International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus (and adeno-associated virus) or alphavirus, all well known in the art.

In a preferred aspect, certain disorders (e.g., of proliferation, such as cancer, etc), can be amenable to treatment by administration of a therapeutic agent based on the provided polynucleotide or corresponding polypeptide. The therapeutic agent can be administered in conjunction with one or more other agents including, but not limited to, receptor-specific antibodies and/or other agents (e.g., chemotherapeutic agents, etc). Administered “in conjunction” includes administration at the same time, or within 1 day, 12 hours, 6 hours, one hour, or less than one hour, as the other therapeutic agent(s). The compositions may be mixed for co-administration, or may be administered separately by the same or different routes.

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents of the invention includes local or systemic administration, including injection, oral administration, particle gun or catheterized administration, and topical administration. The therapeutic polynucleotide composition can contain an expression construct comprising a promoter operably linked to a polynucleotide encoding a Mig-7 polypeptide. Various methods can be used to administer the therapeutic composition directly to a specific site in the body. For example, an abnormal tissue, or small metastatic lesion is located and the therapeutic composition injected several times in several different locations within the body of the tissue, or tumor. Alternatively, arteries which serve a tissue or tumor are identified, and the therapeutic composition injected into such an artery, in order to deliver the composition directly into the tumor. A tissue or tumor that has a necrotic center is aspirated and the composition injected directly into the now empty center of the tissue or tumor. X-ray imaging is used to assist in certain of the above delivery methods.

Mig-7 polypeptide, or anti-Mig-7 antibody-mediated targeted delivery of therapeutic agents to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338.

For gene therapy, therapeutic compositions containing a polynucleotide are administered in a range of about 100 ng to about 200 mg of DNA for local administration in a gene therapy protocol. Concentration ranges of about 500 ng to about 50 mg, about 1 mg to about 2 mg, about 5 mg to about 500 mg, and about 20 mg to about 100 mg of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the subgenomic polynucleotides. Where greater expression is desired over a larger area of tissue, larger amounts of subgenomic polynucleotides or the same amounts re-administered in a successive protocol of administrations, or several administrations to different adjacent or close tissue portions of, for example, a tumor site, may be required to affect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5, 219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. 264:16985 (1989)); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell. Biol. 14:2411 (1994), and in Woffendin, Proc. Natl. Acad. Sci. (1994) 91:11581-11585.

Further non-viral delivery suitable for use includes mechanical delivery systems such as the approach described in Woffendin et al., Proc. Natl. Acad. Sci. USA 91(24):11581 (1994). Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The present invention will now be illustrated by reference to the following examples which set forth particularly advantageous aspects. However, it should be noted that these aspects are illustrative and are not to be construed as restricting the claimed invention in any way.

### EXAMPLES

### Example 1

### MIG-7 was Shown to be a Specific Anti-Cancer Therapeutic Target

### Example Overview

As shown in this Example, using Mig-7 as a target decreases chemoinvasion and enhances stimulation of Human peripheral blood monocytes (MC) to produce cells that lyse MCF-7 tumor cells, and to produce TNF-α in vitro. Enhanced killing indicates that Mig-7 is a tumor antigen target of stimulated human MC, and thus provides proof that Mig-7 possesses tumor specific epitopes recognized by human MC. In addition, these experiments show that Mig-7 peptides combined with MUC1 are superior in stimulating MC to kill MCF-7 cells. Furthermore, Mig-7 peptides, but not irrelevant peptides, enhance production of TNF-α, an MC cytokine known to cause tumor cell death (Bonta and Ben Efraim. Immunology Letters 1990; 25:295-301).

These results additionally indicate that inhibition of Mig-7 expression by antisense or antibody treatment is suitable to inhibit carcinoma cell invasion in vivo.

In addition, peptides to Mig-7 may serve to enhance immunotherapies. Recently it has been shown that vaccine to human papilloma virus strains that prevent cervical cancer are virtually 100% efficacious (Hildesheim et al., J Natl Cancer Inst 2006; 98:292-3). According to additional aspects of the present invention, Mig-7-specific peptides provide novel vaccine compositions, and inoculating with such Mig-7-specific peptide-based vaccines provides novel methods for pan-cancer inoculation and treatment. In addition, Mig-7 peptides may be used ex vivo to stimulate immune cells that can then be infused into a patient for carcinoma cell killing in vivo.

These data are shown, inter alia, to demonstrate the efficacy and importance of targeting Mig-7 in vivo.

### Methods:

Cell Cultures

HEC1A (Crouch et al. Experimental Cell Research 2004; 292:274-87) and MCF7 (Wright et al. Journal of Immunotherapy 2000; 23:2-10) cells were cultured as previously described. Human isolated MC were cultured at 2×10⁶ cells/ml in AIM-V® serum-free lymphocyte medium (Gibco BRL, Life Technologies, Inc. Grand Island, N.Y., USA) as previously described (Wright et al. Journal of Immunotherapy 2000; 23:2-10).

Modified Boyden Chamber Invasion Assay

Chemoinvasion assays were preformed as previously described (Hendrix et al. Invasion & Metastasis 1989; 9:278-97). Briefly, Costar transwell filters (8 μm, top and bottom sides) were blocked in 1% BSA-DMEM/F12 for 30 minutes (600 μL in bottom well, 100 μL in top well) and rinsed once with PBS. Matrigel (BD Biosciences) was diluted in ice cold PBS to 1000 μg/mL. The Bottom well side of pre-chilled transwell insert filters was coated with 30 μL of 1000 μg/mL Matrigel and incubated at 37° C. for one hour. Cells were detached using trypsin and neutralized with soybean trypsin inhibitor. Cells were centrifuged for 5 minutes at 1000 RPM (4° C.) then washed one time in DMEM/F12. Cell count and viability were determined using trypan blue and a hemacytometer. For antisense and sense treatment, cells had been transfected with FuGene6 coupled to oligonucleotides (3:1.5 optimized ratio) for 2 days prior to plating in the well. For antibody experiments, cells were preincubated with 10 μg/mL affinity purified Mig-7 polyclonal antibody (Phillips and Lindsey. Oncology Reports 2005; 13:37-44) for 15 minutes in a 37° C. incubator. Media containing chemoattractant hepatocyte growth factor (HGF) was added to bottom wells first. Media without HGF and containing 50,000 cells was added to top wells. Cells were allowed to invade for indicated times at 37° C. in 5% CO₂, 95% humidified air incubator. After invasion for 72 hours, filters were rinsed with PBS then fixed in Hema-3 fixative for at least 30 minutes. Non-invaded cells in the upper chamber were removed with a cotton swap. Media from bottom wells was also analyzed for cells that potentially invaded through the Matrigel. Filters were dried and stained with Hema3 stains (Fisher Scientific, Inc). Filters were mounted shiny side up using DPX. Invaded cells were counted using a gridded coverslip (Electron Microscopy Science) at 400× magnification with a count of 10 squares (0.6×0.6 mm each) per filter per treatment. All treatments were performed in triplicate and experiments were repeated three times.

Immunoblot

Cell lysates and immunoblots were performed as previously described (Crouch et al. Experimental Cell Research 2004; 292:274-87) with the following modifications. Cells were lysed in 2% SDS, 60 mM Tris, 10% glycerol, 2× protease inhibitor) and quantified using a RC/DC Protein Assay (Bio-Rad). Lysates were boiled for 5 min in the presence of 100 mM DTT and equal amounts of protein were loaded onto a 12% polyacrylamide gel and run at constant 200 V for 30-40 min. Gels were semi-dry transferred (Boekel). Membranes were blocked in TBS-tween (0.05%) containing 5% dry milk for one hour at RT. Endogenous Mig-7 protein was detected using affinity purified Mig-7 antibody (1:2,000). A HRP-labeled secondary anti-rabbit IgG antibody was used to detect the Mig-7 antibody at a dilution of 1:40,000. Anti-β-tubulin (clone AA2, Upstate, Inc.) was used at 1:5000 dilution. Chemiluminescence Plus Reagent (Amersham) allowed detection of HRP-labeled antibodies when exposed to film.

RNA Isolation and RT-PCR

Isolation of total RNA from MCF-7 endometrial carcinoma cells, DNAsing and RT-PCR was performed as previously described (Crouch et al. Experimental Cell Research 2004; 292:274-87).

Peptides

Irrelevant and Mig-7 peptides (see Table 3 for sequences) were synthesized by Biosource, Inc. Peptides were evaluated by mass spectrometry and solubilized in media. Previously characterized MUC1 peptide GNNAPPAHGVNNAPDNRPAP (Wright et al. Journal of Immunotherapy 2000; 23:2-10) was synthesized by American Peptide Co., Inc.

TABLE 3
Peptides used to stimulate MC

| Mig-7 | |
|---|---|
| (Accession DQ080207) | |
| (SEQ ID NO: 1) | Irrelevant |
| RVHMRACSAGSAYLKQMK; | MAASRCSGLYIVRNDTSG; |
| (SEQ ID NO: 2) | (SEQ ID NO: 5) |
| GSAYLKQMKFCRMAASLD; | YIVRNDTSGLSGSQWVDS; |
| (SEQ ID NO: 3) | (SEQ ID NO: 6) |
| FCRMAASLDKVKKTDRGERG. | LSGSQWVDSPLKSPCQVW. |
| (SEQ ID NO: 4) | (SEQ ID NO: 7) |

Stimulation of Human Monocyte Cells and MCF-7 Killing Assay.

Human peripheral blood monocyte cell (MC) isolation, culture, and stimulation, were performed as previously described (Wright et al. Journal of Immunotherapy 2000; 23:2-10.). Briefly, MC were isolated from breast adenocarcinoma patients under IRB approval (Texas Tech University). MC were cultured at 2×10⁶ cells/ml in AIMV® serum-free lymphocyte medium (Gibco) in a 37° C. humidified 5% CO₂, 95% air atmosphere. IL-2 (Cetus) was added twice per week at 100 IU/ml on days 4, and 7. MC were stimulated with 1 μg/ml MUC1 alone or Mig-7 and MUC1 peptides on days 4 and 7. Cell lysis was evaluated on day 8 of peptide stimulation using a tetrazolium salt XTT assay (Roche, Inc) as previously described (Roehm et al. Journal of Immunological Methods 1991; 142:257-65). MCF7 cells (5×10³ per well) were plated into 96-well tissue culture plates. Effector peptide-stimulated MC were added to each well in three effector cell to target cell ratios (E:T): 10:1, 5:1, and 2.5:1. Six wells were plated with MCF7 cells or no cells (background). Treatments were in replicates of 6 and each experiment was performed at least twice.

Maximal XTT™ was determined as the mean of six wells containing target MCF7 cells (i.e. no MC) and background was determined as the mean of the six wells containing only medium. The specific formation of formazan attributable to the presence of effector MC cells was determined from the wells containing effector cells alone. The percent specific lysis (% SL) was calculated as follows as previously described (Wright et al. Journal of Immunotherapy 2000: 23:2-10):

$$\% SL = \frac{{OD}_{( target - medium )} - {OD}_{( experimental wells - well with corresponding number of effector )}}{{OD}_{( target - medium )}} \times 100$$

Day 0 represents MCs that were not stimulated with peptides.

ELISA Cytokine Assay

The ELISA cytokine assay was a solid phase sandwich Enzyme Linked-Immuno-Sorbent Assay (ELISA) (BD Pharmingen Inc), used for the quantitative determination of human cytokines tumor necrosis factor-alpha (TNF-α), interferon-gamma (IFN-γ), and interleukin-10 (IL-10). Amounts of each cytokine present in supernatants were determined by measuring absorbance with a spectrophotometer, according to manufacturer's instructions.

Statistical Analysis

Determinations of the statistical significance of the in vitro cytotoxicity assays and cytokine assays were performed by the Mann-Whitney Rank Sum test. Data from invasion assays were statistically analyzed by one way ANOVA and considered significant at 0.05.

### Results:

Decrease of Mig-7 Protein Resulted in Decreased Carcinoma Cell Invasion

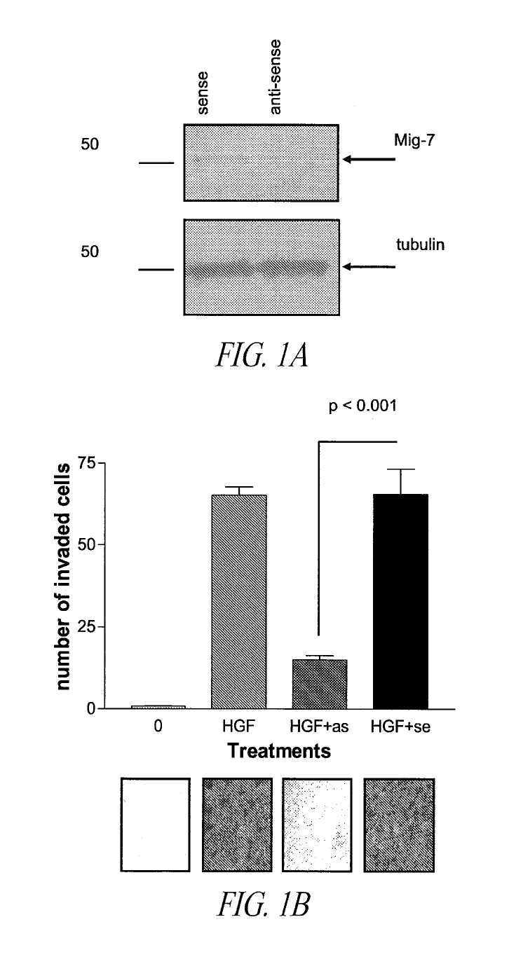

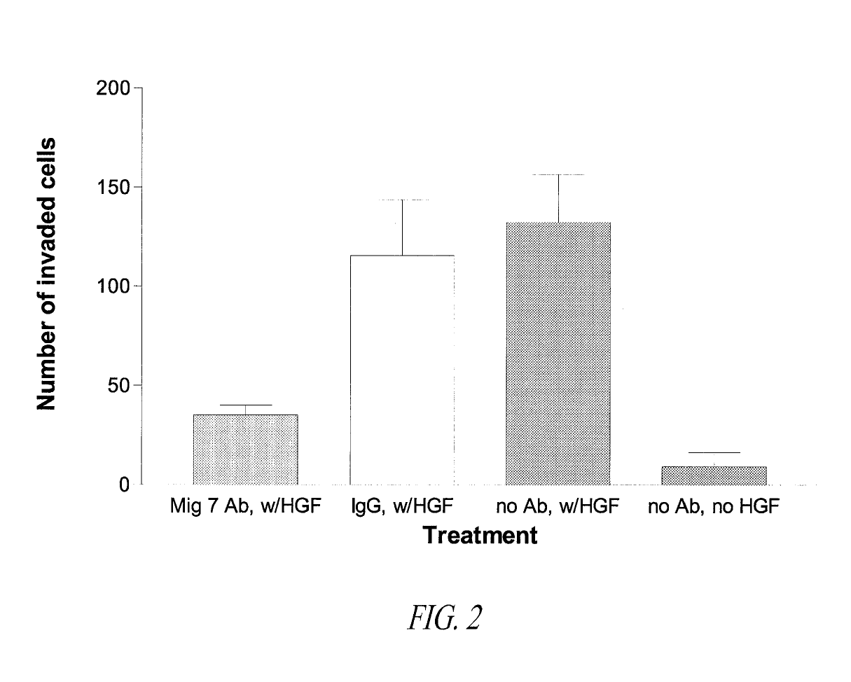

Because Mig-7 antisense oligonucleotides inhibit cell scattering of cells plated for at least four days in a scratch assay of cell migration (Crouch et al. Experimental Cell Research 2004; 292:274-87), these same oligonucleotides (ODN) were used to test if they decreased Mig-7 protein expression and if they inhibit cell invasion. In vitro invasion assays predict the invasive capability of cells in vivo. In addition, stably transfected HT29 cells expressing Mig-7 invade in 3-D Matrigel cultures. Mig-7 antisense but not sense decreased Mig-7 protein levels by 60% as determine by immunoblot (FIG. 1B).

Using a modified Boyden chamber assay of cell invasion as described in Methods and Materials, Mig-7 anti-sense or sense treated HEC1A endometrial carcinoma cells were placed in the upper chamber of transwell (n=3 each treatment) on which the bottom side had been coated with Matrigel. Chemoinvasion of HEC1A cells toward HGF in the bottom wells were significantly inhibited by 3.5-fold (p<0.001) as compared to HGF alone or HGF with sense ODN (FIG. 1B).

FIGS. 1A and 1B show, according to exemplary aspects of the present invention, that antisense to Mig-7 inhibits chemoinvasion. A) Immunoblot demonstrating that Mig-7 antisense ODN decreases Mig-7 protein levels in lysates from HEC1A cells treated as described in Materials and Methods as compared to lysates from HEC1A cells treated with Mig-7 sense ODN, B) Mig-7 antisense (as), significantly inhibits HEC1A carcinoma cell SF(HGF) chemoinvasion of Matrigel as compared to SF (HGF) treatment alone or SF (HGF) with sense (se) Mig-7 ODN. Images below each treatment group show a representative invasion of cells for that group. Experiments were performed in triplicate for each treatment and repeated three times.

Antibody to Mig-7 Resulted in Decreased Invasion In Vitro

Antibodies have previously been shown to inhibit invasion of carcinoma cells expressing another membrane protein, chemokine receptor CXCR4, expressed on cancer cells however this expression is also found on normal cells (Müller et al. Nature 2001; 410:50-6). Therefore, the Applicant used affinity purified antibody to a peptide representing the first nine amino acids of Mig-7 (SEQ ID NO: 8) which is the encoding region beginning at the consensus Kozak ATG site and ending at the first stop codon which is read-through during translation). This antibody detects endogenous Mig-7 at the same size as anti-FLAG antibody detects FLAG-tagged Mig-7 in stably transfected cells. Using this Mig-7 antibody to treat HEC1A cells before plating in the upper well of the modified Boyden chamber as described in the Methods section, a significantly decreased number of invaded cells was observed. When compared to irrelevant antibody, chemoinvasion toward HGF in the lower well was significantly inhibited (−80.4, p value 0.0046) by Mig-7 antibody (FIG. 2). Irrelevant IgG antibody did not significantly inhibit chemoinvasion whereas HGF significantly enhanced chemoinvasion over no HGF (FIG. 2).

FIG. 2 shows, according to exemplary aspects of the present invention, that antibody specific to Mig-7 inhibits HEC1A endometrial carcinoma chemoinvasion. Mig-7 affinity purified antibody but not IgG isotype antibody significantly decreased chemoinvasion of HEC1A cells into Matrigel. Experiments were performed in triplicate for each treatment and repeated three times.

Stimulation of Human Peripheral Blood Monocytes (MC) with Peptides Specific to Mig-7 Enhanced Killing of MCF-7 Breast Carcinoma Cells In Vitro.

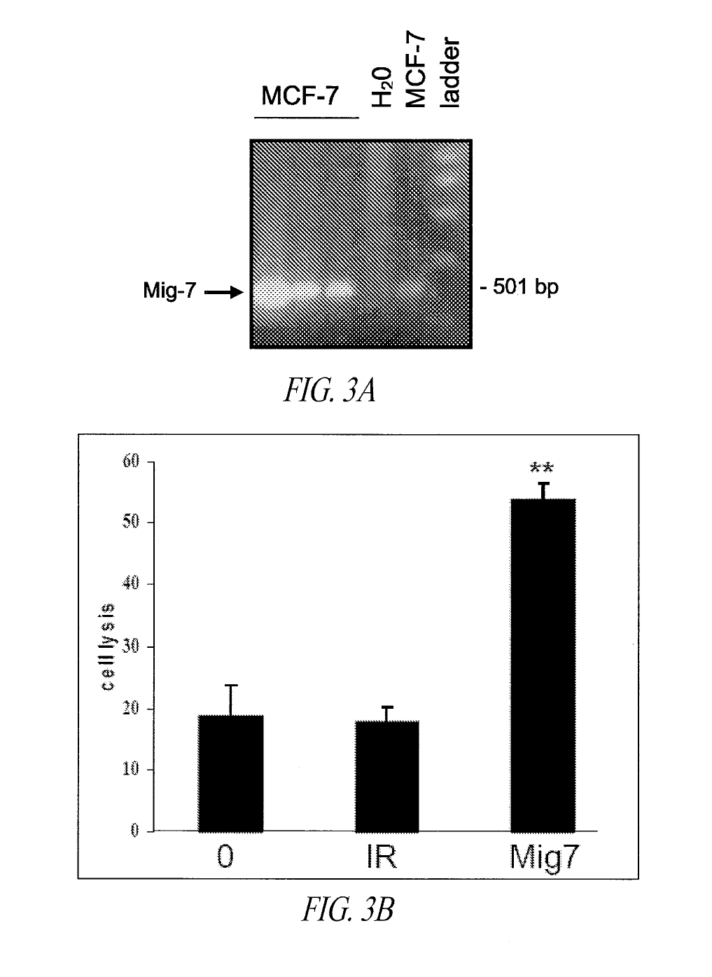

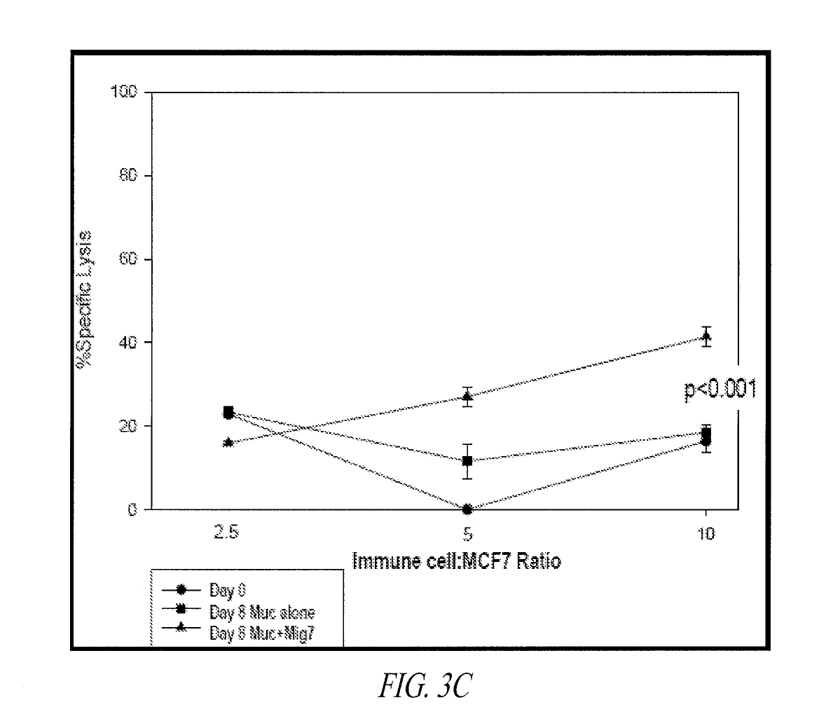

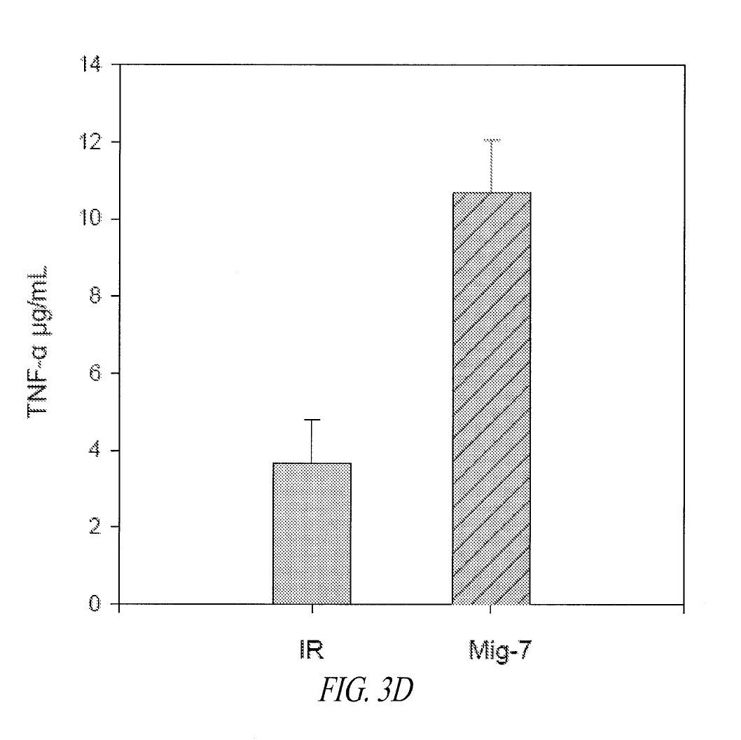

Human MC were isolated under IRB approval as previously described (Wright et al. Journal of Immunotherapy 2000; 23:2-10). Mig-7 or irrelevant peptides (Table 3, herein above) were used first to determine which sequences contributed to enhanced MCF-7 killing in vitro. MCF-7 cells, as do most carcinoma cells, express Mig-7 (FIG. 3A). Peptides used are listed in Table 3. Stimulation with Mig-7 peptides significantly enhanced MC killing of MCF-7 cells by >2-fold over MUC1 peptide alone or irrelevant peptides. There was no significant difference between irrelevant peptides and MUC1 peptide alone (FIG. 3B At a ratio of 10 MC to 1 MCF-7 cell, unstimulated and MUC1 stimulated were indistinguishable in levels of MCF-7 killing. In contrast, Mig-7 peptides significantly increased MC killing of MCF-7 cells >2-fold over unstimulated and MUC1 stimulated as well as 1.9-fold over Muc1 with Mig-7 at the 5:1 ratio (FIG. 3C). Mig-7 peptide also significantly increased levels of MC produced tumor necrosis factor-α (TNF-α) over irrelevant peptide (FIG. 3-D).

FIGS. 3A-D show, according to exemplary aspects of the present invention, that Mig-7 peptides enhance human monocyte killing of MCF-7 breast carcinoma cells. (A) Representative RT-PCR demonstrating expression of Mig-7 in MCF-7 cells. (B) Human MC cells stimulated with IL-2 and either no peptide (O), pooled irrelevant peptides (IR), or pooled Mig-7 peptides. (see Table 3). Note that Mig-7 peptides significantly (p value 0.001) enhanced MC killing of MCF7 carcinoma cells. Experiments have been repeated 3 times in replicates of 6 for each treatment group. MC were isolated from two different individuals. (C) Cytotoxic response of human isolated MC after peptide stimulation using indicated ratios of immune cells (MC) to MCF-7 cells. Bars are ±S.E. Experiments were repeated twice with replicates of six for each treatment group. (D) TNF-α production by human isolated MC after indicated peptide stimulation. TNF-α production was measured by ELISA assay as described in Methods and Materials after MC were cultured for 8 days with MUC1 peptide plus irrelevant peptides Mig-7 peptides. Bars are ±S.E. Assays were performed in triplicate. The mechanism by which stimulation with Mig-7 peptide enhances killing of MCF-7 cells is likely due to TNF-α because this cell line is sensitive to the cytokine (Xiang et al J. Biochem. 53:3-12 (1997)).

### Example 2

### Translation of the Human Carcinoma- and Trophoblast-Specific Mig-7 Requires Fidelity of the Purine-Pyrimidine Repeat Region and Genetic Recoding

### Example Overview

As stated above, Migration inducting gene-7 (Mig-7) is a new, human oncogene that is expressed strictly by solid tumor cells and by invasive fetal trophoblast cells but not by normal tissues. In 3-D cultures, expression of Mig-7 in a noninvading carcinoma cell line is sufficient to cause sensing of the microenvironment, invasion and formation of vessel-like structures with a lumen. This expression also causes transfected cells to be significantly less adherent to tissue culture plastic and to laminins. Furthermore, antisense or antibody to Mig-7 inhibits migration and invasion in vitro. Receptor tyrosine kinases in concert with ligation of αvβ5 integrin induce Mig-7 mRNA but cloning and consistent endogenous protein expression as well as detection of Mig-7 protein requires tumor microenvironment factors.

Cloning of genes is now usually routine in molecular biology. Genetic sequences that allow cloning, expression and detection by standard molecular techniques are most commonly studied, leaving the function of the most difficult to clone and detect of these genetic codes a mystery.

For example, in this Example 2, Applicant demonstrates, surprisingly, that a cancer-specific, “noncoding” mRNA produces protein when using Applicant's combination of techniques. Migration inducting gene-7 (Mig-7) is unique in its induction, translation and detection, and is highly cancer cell-specific. Research into this important oncogene has been hampered by the non-classical molecular and cell culture techniques required for cloning, maintenance of sequence fidelity, and translation. For example, genetically engineered E. coli are required to maintain the number of Mig-7 purine-pyrimidine repeats and reading frame when producing plasmids for expression. A Mig-7 sequence that produces protein in vivo contains stop codons. Nonetheless, the Applicant has confirmed herein that the Mig-7 sequence produces protein in vivo from the conserved Kozak consensus site ATG and not from other upstream start codons. Furthermore, mRNA tertiary structures reside in appropriate locations with respect to the first stop codon that likely allow recoding and frameshifting to produce Mig-7 protein in vivo.

In this Example 2, the Applicant details novel requirements for Mig-7 cloning and expression as well as relationship of these techniques to tumor progression. These techniques help elucidate the unique, tumor- and fetal trophoblast-specific expression for use as a novel biomarker (e.g., diagnostic, and/or prognostic marker) and as a therapeutic target for cancer therapies.

Methods for Cloning and Expressing an Atypical Gene with Qt Repeats and Stop Codons, and Results:

I. Fidelity of Mig-7 Purine-Pyrimidine Repeat Region Requires Genetically Engineered E. Coli that Lack Rearrangement Capability:

During initial isolation of Mig-7 cDNA, the Applicant determined a guanine-thymine (gt) repeat region that encodes cysteine-valine dipeptide repeats (Crouch et al. Experimental Cell Research 2004; 292:274-87) that varied in number of gt repeats when grown in TOP10F′ E. coli. It is known that Purine-pyrimidine repeats, resulting in a left handed zig-zag configured DNA (z-DNA), can be rearranged in sequences due to recombination events in E. coli (Santella et al. Proceedings of the National Academy of Sciences USA, 1981; 78, 1451-1455; Fuchs et al. Methods and Consequences of DNA damage processing. Alan R. Liss, Inc.: New York 1988). Therefore, to preclude unwanted rearrangements in E. coli, a genetically engineered strain from Stratagene, or TOP10F′ E. coli was used for transformation with 3XFLAGCMV vector containing the Mig-7 cDNA as described herein.

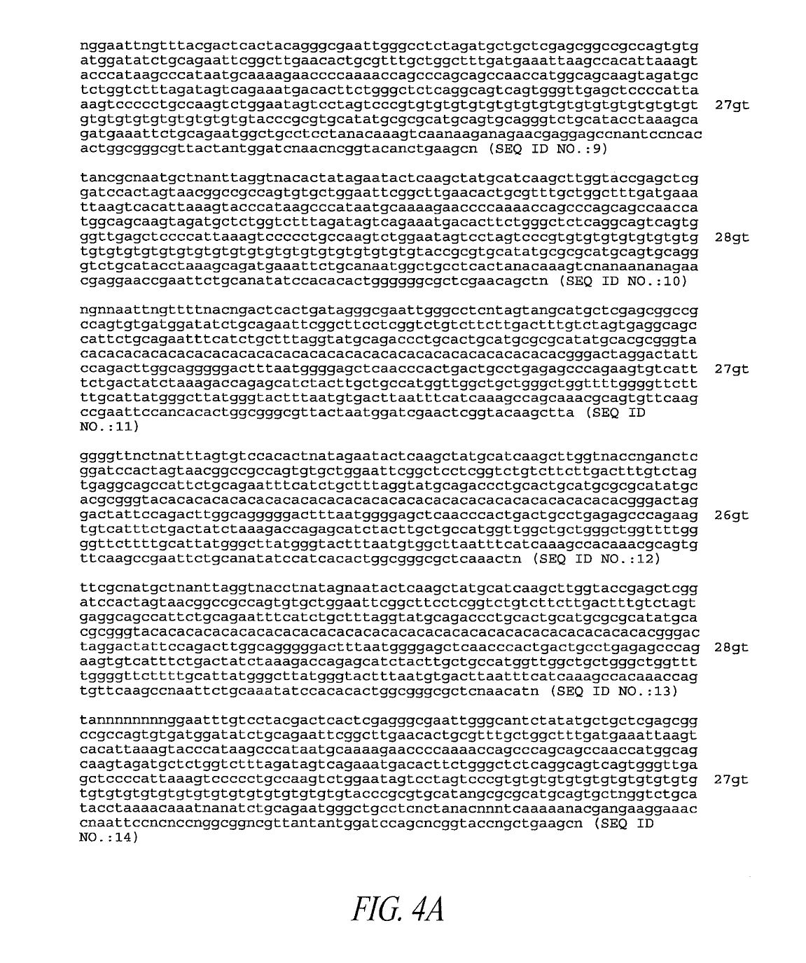

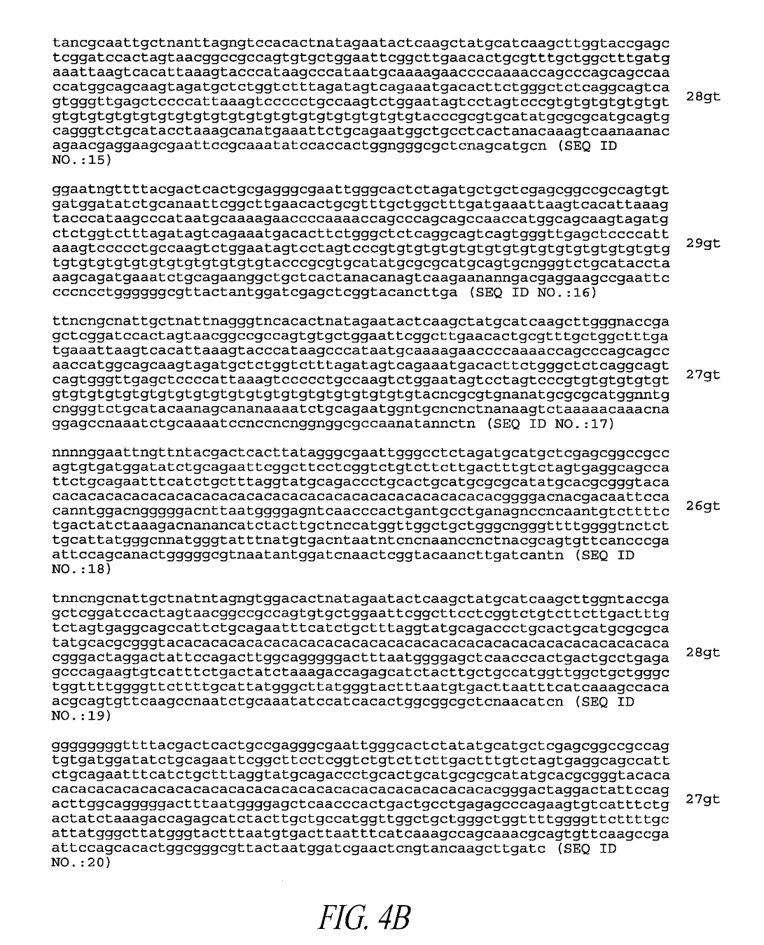

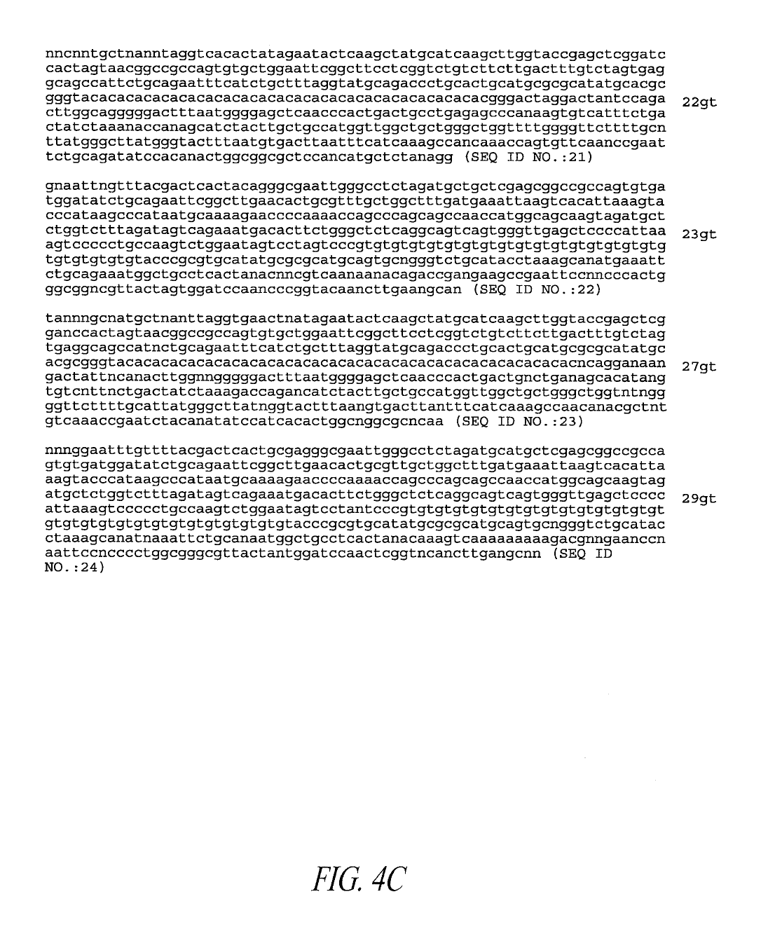

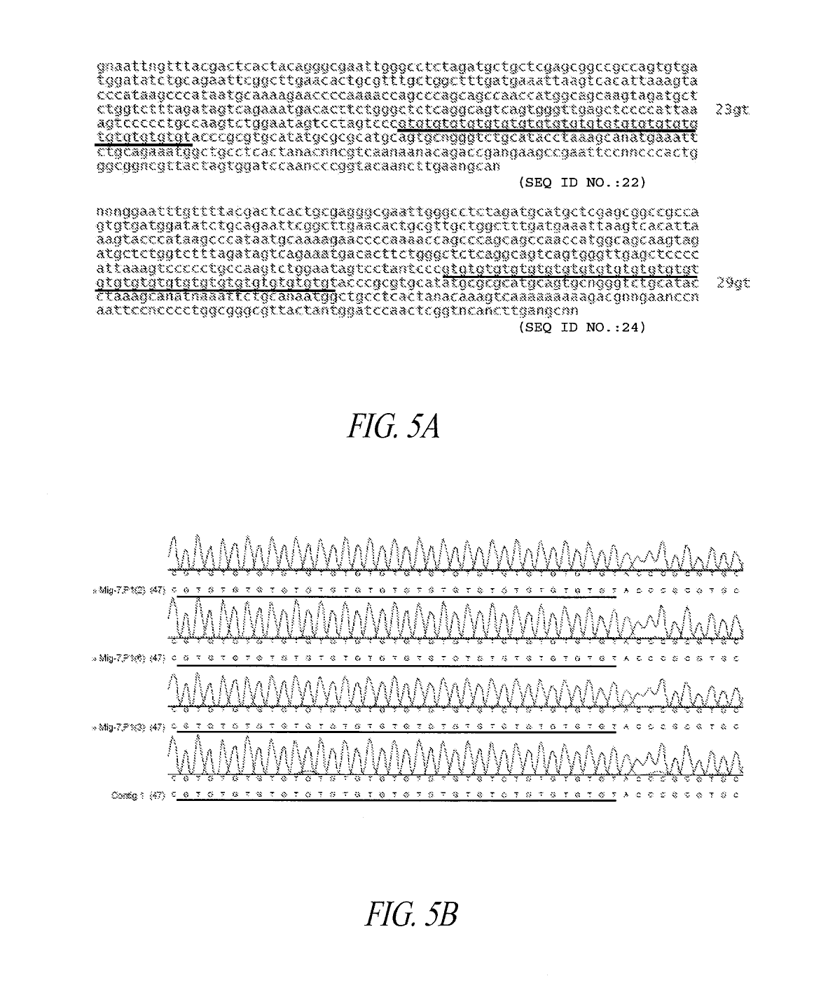

FIGS. 5A and 5B show that genetically engineered E. coli (SURE™, Stratagene) are required (suitable) to maintain the integrity of the Mig-7 purine-pyrimidine repeat coding region. The Mig-7 sequence was amplified using primers corresponding to 805 to 1529 (SEQ ID NOS: 29, 30, respectively) (Table 4) of the Mig-7 sequence (Accession number DQ080207) (SEQ ID NO:1). After amplification and checking size on a gel, fragments were cut with the indicated restriction enzymes, gel purified and ligated into p3XFLAG-CMV vector (SIGMA) that was also digested with HindIII and BamHI enzymes for directional insertion. TOP10F′ E. coli or SURE cells were transformed using the chemical heat shock method and allowed to incubate in SOC media for one hour prior to plating on LB ampicillin (100 μg/ml) plates. At least 16 clones were isolated for each E. coli strain grown in LB broth with 2% glucose and plasmids purified using Qiagen Endo-free kit according to manufacturer's instructions. After purification, plasmids were sequenced at the Texas Tech sequencing facility and compiled using VectorNTI software program. FIG. 5A shows representative sequences of Mig-7 region containing gt repeats (underlined) ranging from 23 (SEQ ID NO: 22) to 29 (SEQ ID NO: 24) repeats in length (additional sequences are shown in FIG. 4). FIG. 5B shows Representative sequences of Mig-7 plasmids grown in SURE cells. Note the consistent number (18) of gt repeats.

TABLE 4
Mig-7 specific targets used for cloning

| Region of | Construct | |
|---|---|---|
| DQ080207 | ID | Primer pair |
| 110-1529 | 1, 0 | (F)5′GCGCAAGCTTTATATGATGCCCC |
| | | ACC CAG3′ |
| | | (R)5′GCGCGGATCCGCCCGTGATGAAT |
| | | CATGTGAC 3′) |
| | | (SEQ ID NOS: 25, 26, |
| | | respectively) |
| 531-1529 | 1, 16.3 | (F)5′GCGCAAGCTTCCCATGTCACAGT |
| | | CCAGGCA3′ |
| | | (R)5′GCGCGGATCCGCCCGTGATGAAT |
| | | CATGTGAC 3′) |
| | | (SEQ ID NOS: 27, 28, |
| | | respectively) |
| 805-1529 | 1, 16.2 | (F)5′GCGCAAGCTTCAGCCAACCATGG |
| | | CAGC A 3′) |
| | | (R)5′GCGCGGATCCGCCCGTGATGAAT |
| | | CATGTGAC 3′) |
| | | (SEQ ID NOS: 29, 30, |
| | | respectively) |

F = forward, R = reverse

It was also found, at least in the context of 3XFLAGMig-7 or empty vector, the growth of cells required additional glucose (2%, Luria broth). FIG. 5 contains examples of Mig-7 sequences transformed in and grown in these two different E. coli strains and the altered length of Mig-7 gt repeats underlined (additional sequences are shown in FIG. 4). Guanine-thymine repeats varied from 23 to 29 in length in TOP10F′ Mig-7 clones (FIG. 5A) in contrast to a consistent length of 18 repeats in Mig-7 sequences from SURE cell clones (FIG. 5B). In addition, translation from vectors with extended gt repeat regions did not express Mig-7 protein in vivo demonstrating the importance of maintaining this region for Mig-7 expression.

Whether cloning for genomic sequencing or for functional expression of cloned cDNA, using E. coli without rearrangement capabilities may lead to protein expression from sequences bearing purine-pyrimidine repeats. For example, the EST (N41315) that this region of Mig-7 is homologous to (Crouch et al. Experimental Cell Research 2004; 292:274-87) contains 19 instead of 18 repeats (Genbank), and thus, would not produce protein. Furthermore, BLAST searches (Altschul et al. 1997) revealed over 850 hits on sequences containing gt repeats. Mig-7 cloning is just one example of a sequence with a region that can be rearranged and use of this genetically engineered E. coli should be used more often if functional studies of genes containing such repeats as well as other sequences that can be rearranged or deleted are to be reliable and consistent.

II. 3XFLAGMig7 Produces Protein In Vivo from Kozak ATG:

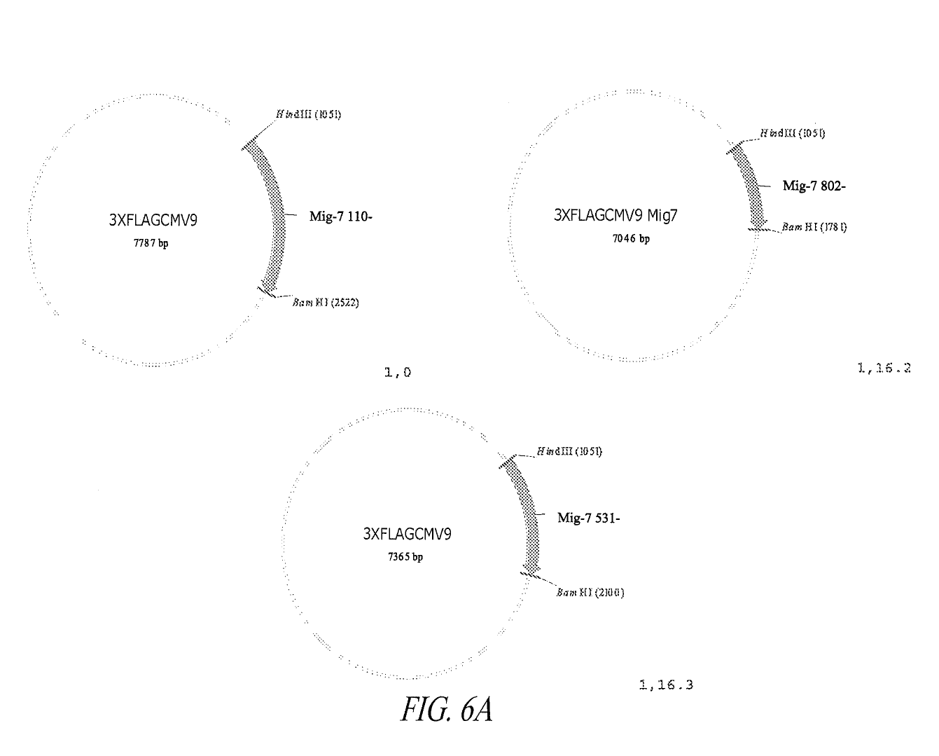

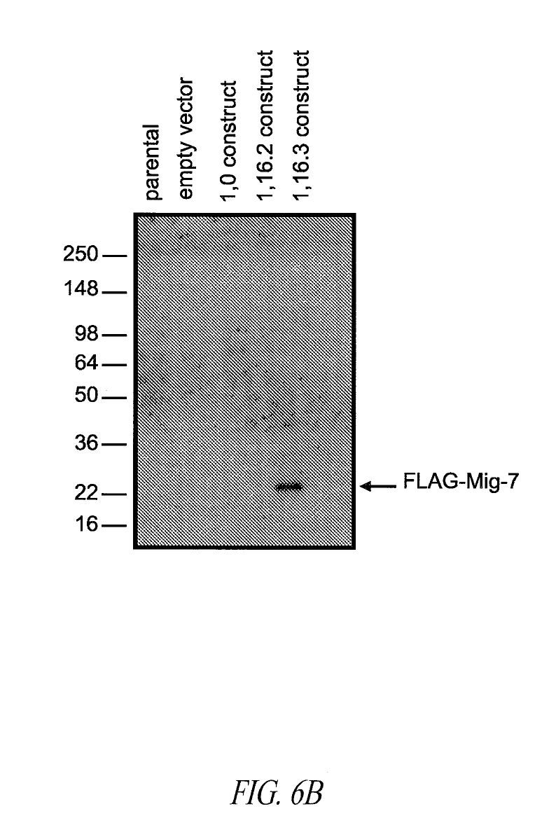

Because of the unusual encoding and 5′ regions of Mig-7 (accession DQ080207) (SEQ ID NO:1), several ATG sites representing three different reading frames were cloned as detailed herein for functional expression studies (FIG. 6).

FIGS. 6A and 6B show that Mig-7 cloning into 3XFLAG-CMV produces protein from the Kozak consensus site ATG and not from other upstream ATG sites. FIG. 6A shows that all three constructs were cloned as described in FIG. 5 legend except using primer pairs in Table 4. Vectors were drawn in VectorNTI. Empty vector or Mig-7 containing vectors were transfected (3:1 optimized ratio FuGene6, Roche) into HT29 colon carcinoma cells (ATCC) cultured in McCoy's 5a medium with 10% FCS at 37° C. and 5% CO₂. Thirty-six hours after transfection, cells were selected in culture medium containing 800 μg/ml G418. After 21 days of selection, individual G418-resistant colonies were pooled for each well (n=3). FIG. 5B shows a representative immunoblot of protein lysates from HT29 cells that do not endogenously express Mig-7 due to a lack of αvβ5 integrin (Crouch et al. Experimental Cell Research 2004; 292:274-87) and that were individually stably transfected with the vector constructs described in FIG. 6A. Briefly, protein lysates (2% SDS, 60 mM Tris, 10% glycerol) were quantified using RC/DC Protein Assay (Bio-Rad). After heating samples for 5 min at 90° C. in the presence of 100 mM DTT, equal amounts of protein were loaded onto a 4-20% gradient gel and electrophoresed at constant 200 V for 30-40 min. Gels were equilibrated in transfer buffer and semi-dry transferred (Boekel) for 1.5 h at 25 mAmp. Membranes were blocked in TBS-tween (0.05%) containing 5% dry milk for one hour at room temperature. FLAG-tagged (amino terminus) Mig-7 protein was detected using M2-peroxidase anti-FLAG antibody (1:100, Sigma). Chemiluminescence Plus Reagent (Amersham) allowed detection of HRP-labeled antibody. Representative immunoblot of lysates from stably transfected HT29 cells showing that 1,16.2 construct produced FLAGMig-7 protein of the predicted size (23 kD) and this band was absent in 1,0 and 1,16.3 construct protein lysates. Transfections, in triplicate for each construct, and immunoblots were performed at least twice with all three constructs and empty vector.

Thus, after successful growth, we harvested plasmids by Qiagen endo-free midi preparations and performed stable transfections using G418 at 800 μg/ml for selection that was previously determined by cell death curve anlysis of HT29 colon carcinoma cells. This cell line was chosen because it does not endogenously express Mig-7 due to a lack of αvβ5 integrin expression (Crouch et al. Experimental Cell Research 2004; 292:274-87). HT29 cells transformed with the Mig-7 sequence from the Kozak consensus ATG (1,16.2) produced FLAG-Mig-7 protein in HT29 cells (FIG. 6B) whereas construct 1,16.3 and 1,0 did not express protein.

HT29 cells stably transfected with FLAG-Mig-7 (1,16.2, construct) are significantly less adherent to tissue culture plastic and to laminins (see Example 1 and 3 herein). Furthermore, in 2D cultures, there is very little difference in morphology compared to empty vector transfected cells in stark contrast to 3-D cultures where these cells form vessel-like structures with lumens similar to vasculogenic mimicry by tumor cells with aggressive behavior. Vasculogic mimicry by tumor cells is proposed to circumvent angiogenesis and may be why anti-antiangiogenic therapies show limited efficacy (Hess et al. Cancer Res, 65, 9851-9860; Seftor et al. Cancer Res 2001; 61:6322-7; van der Schaft et al. Cancer Res, 65, 11520-11528). Thus, polyadenylated mRNA with unusual reading frames that contain stop codons (see below) should be considered potentially functional to produce protein rather than relying on current algorithms to predict translation.

III. Mig-7 Protein Encoding Sequence Contains Stop Codons:

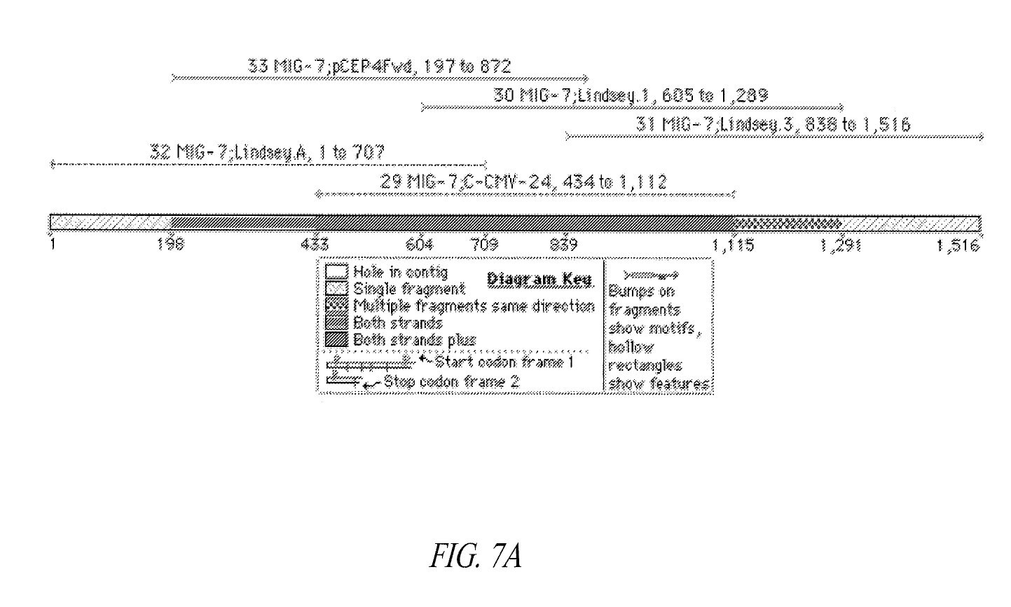

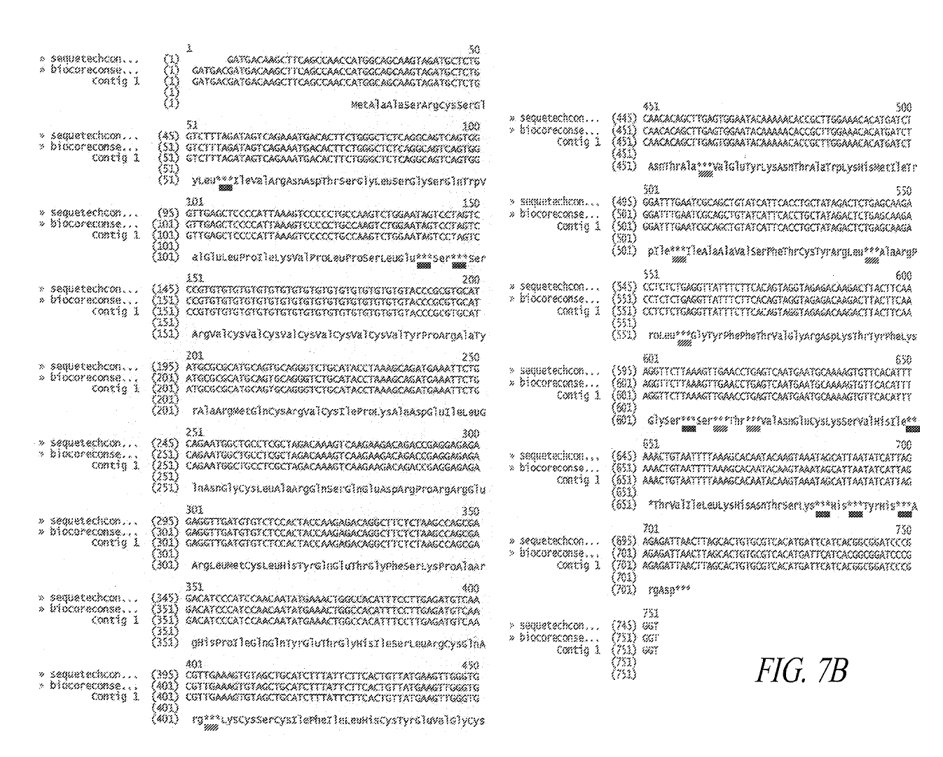

The first rounds of Mig-7 sequencing contained multiple stop codons and this finding was initially considered to be a sequencing mistake, particularly because this cloned region produced protein in HT29 cells (FIG. 6B). However, after sequencing by two, independent facilities, with at least two different primer sets each (FIG. 7A shows a representative sequencing scheme), at least twice at each facility and compiling in VectorNTI, both consensus sequences were 100% homologous and contained stop codons (FIG. 7B). Amazingly, there are 15 stop codons in the “0” reading frame of the region that expresses ˜22 kD protein in HT29 colon carcinoma cells (FIG. 7B). However, with frameshifting +1 and recoding (see below), stop codons would be eliminated and the size of Mig-7 protein observed by immunoblot analyses would be synthesized.

Specifically, FIGS. 7A and 7B show that the Mig-7 sequence that produced protein in vivo contains multiple stop codons in reading frame “0”. Plasmids were produced and grown as previously described in FIG. 5, then sent to two different sequencing facilities, Texas Tech University Sequencing Core and Sequetech, Inc. Both facilities sequenced at least twice using two different overlapping primer sets in both directions each. (a) Representative sequencing scheme of overlapping and complementary strand sequencing. FIG. 7B shows that the consensus sequences from each facility are 100% homologous. Stop codons are indicated with asterisks in the predicted amino acid sequence from reading frame “0”. Green highlights are UGA that can also encode for selenocysteine. Red highlights are UAG or UAA stop codons.

Additional sequence confirmed the presence of three conserved point mutations in Mig-7: a G to C mutation at position 1048 of DQ080207; a C to T mutation at position 1112 of DQ080207; and a C to T mutation at position 1323 of DQ080207.

IV. Mig-7 mRNA Contains Predicted Slippery and Pseudoknot Motifs that Are Known in Other Sequences to Allow Frameshiftinq and Read-Through of Stop Codons:

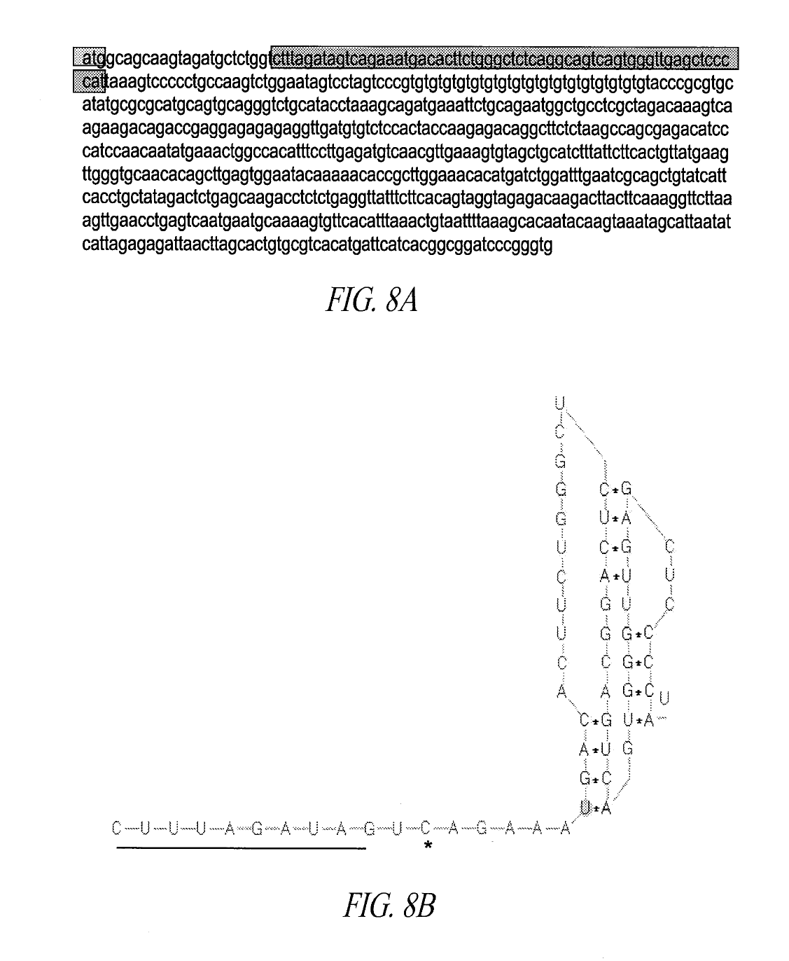

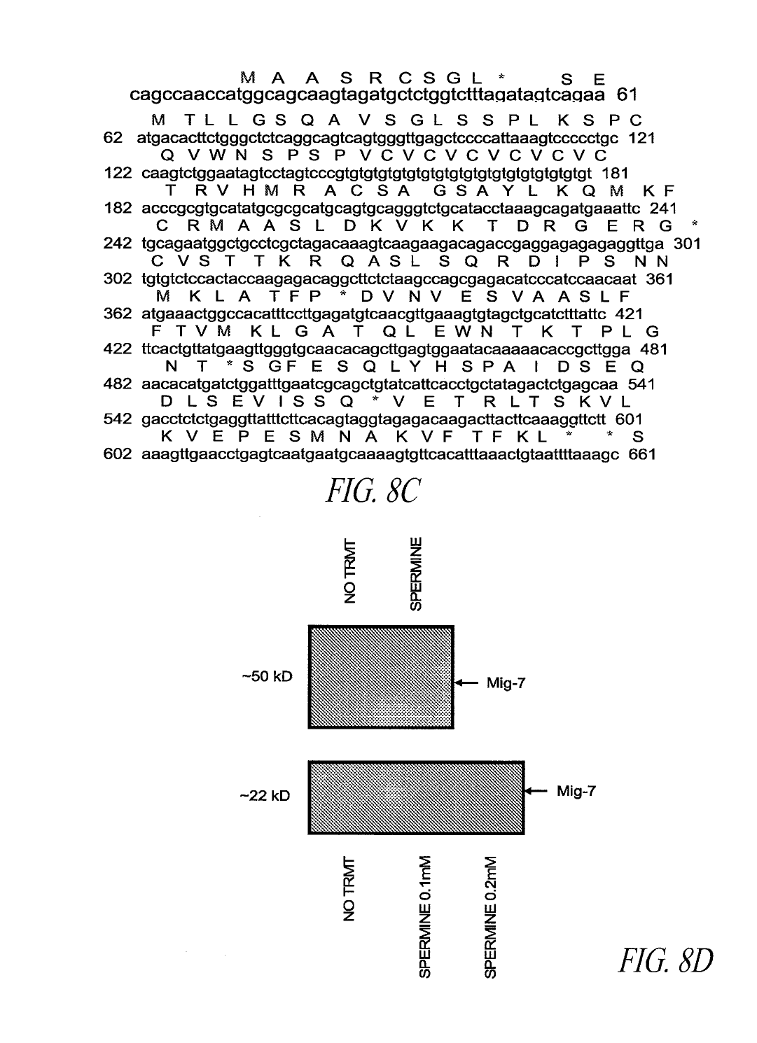

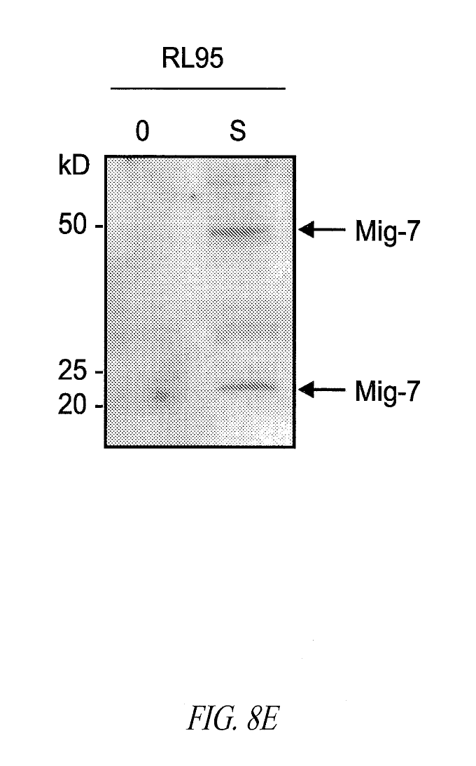

In order to find an explanation for expression of protein from Mig-7 stop codon containing cDNA, the Applicant examined the sequence for possible ‘recoding’ structures. Pseudoknots are one type of tertiary mRNA structure 3′ of stop codons with complementary binding of sequences outside a stem loop that bind to the loop (Giedroc et al. Journal of Molecular Biology 2000; 298, 167-185). This type of structure can stimulate the ribosome to shift −1 or +1 in reading frame at slippery or “shifty” sites, although the exact molecular mechanisms are unclear. While this is a common mechanism of translation through a stop codon in viral transcripts, mammalian genes, Edr and Antizyme (AZ), are known to contain pseudoknots that stimulate frameshifting through a stop codon (Manktelow et al. Nucl Acids Res 2005; 33, 1553-1563; Howard et al. Genes to Cells 2001; 6, 931-941). Using the FSFinder software program (Moon et al. Nucl Acids Res 2004; 32, 4884-4892), a predicted pseudoknot structure was determined in the Mig-7 sequence at a distance and structure consistent with known frameshifting (FIG. 8A). In addition, a predicted slippery site is located 5′ of the first stop codon which would be after translation of the first nine amino acids (FIG. 8A). This pseudoknot is located 11 base pairs 3′ of the first stop codon and is of the H-type (FIG. 8B) as determined by analysis of the highlighted sequence in the PsuedoViewer2 program (Han and Byun. Nucl Acids Res 2003; 31, 3432-3440). The predicted shift site and stop codon are underlined (FIG. 8B) indicating that the stop codon is in the context of a “shifty” site containing three uridine nucleotides typical of +1 frameshifting at the underlined heptanucleotide (Shah et al. Bioinformatics 2002; 18, 1046-1053). Intriguingly, there is another stop codon immediately after the first stop in the +1 frame that we predict is read-through. FIG. 8C shows Mig-7 protein encoding sequence after frameshifting +1. This reading frame has the least number of subsequent stop codons and those are primarily, three out of four, TGA stop codons that can also encode for selenocysteine (Gesteland et al. Science 1992; 257, 1640-1641).

Specifically, FIGS. 8A-8E show that the Mig-7 sequence contains a predicted shift-site and pseudoknot indicating potential frame-shifting, and that Mig-7 protein levels are increased with polyamine treatment consistent with recoding during translation. FIG. 8A shows a Mig-7 sequence from start codon (blue highlight, box) showing a highlighted putative shift site and pseudoknot in context (orange highlight, box). FIG. 8B shows a putative, Mig-7 mRNA pseudoknot structure with shift-site underlined and stimulatory uridine for read-through after the stop codon with an asterisk highlighted in FIG. 8A. FIG. 8C shows a sequence of Mig-7 protein after +1 frame-shift at the predicted shift site. This image also shows that only four additional stop codons are in this reading frame before the most likely terminal stop (the double-stop located between nucleotide positions 632 and 640 of SEQ ID NO:35). The isoelectric point of the 203-207 amino acid frame-shift polypeptide is 8.23 and this sequence has 5 predicted PKC phosphorylation sites, 6 CK2 phosphorylation sites, and 2 myristoylation sites as determined by Prosearch.

Because Mig-7 is highly cysteine rich encoded by the gt repeat region (FIG. 5B), it would not be surprising if the UGA codon is recoded as a selenocysteine. Furthermore, in viral stop codon read-through, the subsequent hexanucleotides have been suggested to play a role, however, this sequence varies (Harrell et al. Nucl Acids Res 2002; 30, 2011-2017). Recoding or read-through of these subsequent stop codons is a possibility, and such products are emcompassed herein. Nevertheless, we consistently observe ˜22 and ˜50 kD sized bands on immunoblots using antibody to the first nine amino acids (SEQ ID NO:31) which encodes for a peptide specific to Mig-7 (Crouch et al. Experimental Cell Research 2004; 292:274-87) or using FLAG antibody to detect transfected FLAG-tagged Mig-7. Observing a larger than predicted size for a given protein on immunoblots has precedence in another cysteine-rich protein, Ribonuclease U2 (Lucia-Ortega et al. Electrophoresis 2005; 26, 3407-3413) and for laminin receptor which is predicted by mRNA to encode a 37 kD protein but is consistently detected at 67 kD. Laminin receptor plays an important role in tumor cell invasion (Givant-Horwitz et al. Cancer Letters 2005; 223, 1-10). Intriguingly, Mig-7 expression causes significantly lower adhesion to laminins. While these data seem contradictive, there are laminin γ-chain fragments produced by aggressive tumor cells that promote their invasion and vasculogenic mimicry (Seftor et al. Cancer Res 2001; 61:6322-7) processes in which Mig-7 plays a role (submitted). It is likely that Mig-7 forms multiple cysteine bonds with either itself or other proteins resulting in the ˜50 kD sized band on immunoblots.

It has been over a decade since it was proposed that all organisms have particular mRNAs that can be recoded (Gesteland et al. Science 1992; 257, 1640-1641). Mig-7 cDNA sequence structure is consistent with this recoding, and such recoded variants are encompassed herein. In addition, reinitiation of translation is more efficient when this takes place close to the initial start codon (Kozak 2001) consistent with the localization of the shift site and pseudoknot in Mig-7 sequence beginning only seven codons downstream of the ATG translation start site (FIG. 8A), and such reintiation variants are encompassed herein.

Intriguingly, Mig-7 genomic sequence, on human chromosome 1 (Crouch et al. Experimental Cell Research 2004; 292:274-87), contains no introns, which is reminiscent of viral protein encoding DNA. In addition, most recoding due to frameshifting is through a single stop codon whereas, Mig-7 has four additional stop codons in the +1 frameshifted sequence including three UGA that can also encode for selenocysteine (Atkins and Gesteland. Science 2002; 296, 1409-1410). Yet, Mig-7 produces protein in vivo (Crouch et al. 2004; Phillips and Lindsey, 2005), FIG. 6B). The Applicant speculates that the predicted Mig-7 pseudoknot stimulates a +1 frameshift due to experiments that showed overlapping peptides specific to this frame stimulate human peripheral blood monocyte killing of MCF-7 breast carcinoma cells in vitro in a statistically significant manner (submitted; also see Example 1 herein). Mig-7 slippery sites 5′ of the stop codon may involve the multiple uridine nucleotides, similar to those that have been shown in yeast, +1 frameshifted, actin filament binding ABP140 and telomerase subunit EST3 sequences (Shah et al. Bioinformatics 2002; 18, 1046-1053). As with another +1 frameshifed protein, AZ, where the mammalian 5′ slippery site is modular (Ivanov et al. Nucl Acids Res 2000; 28, 3185-3196), the Mig-7 5′ element will need to be determined empirically.

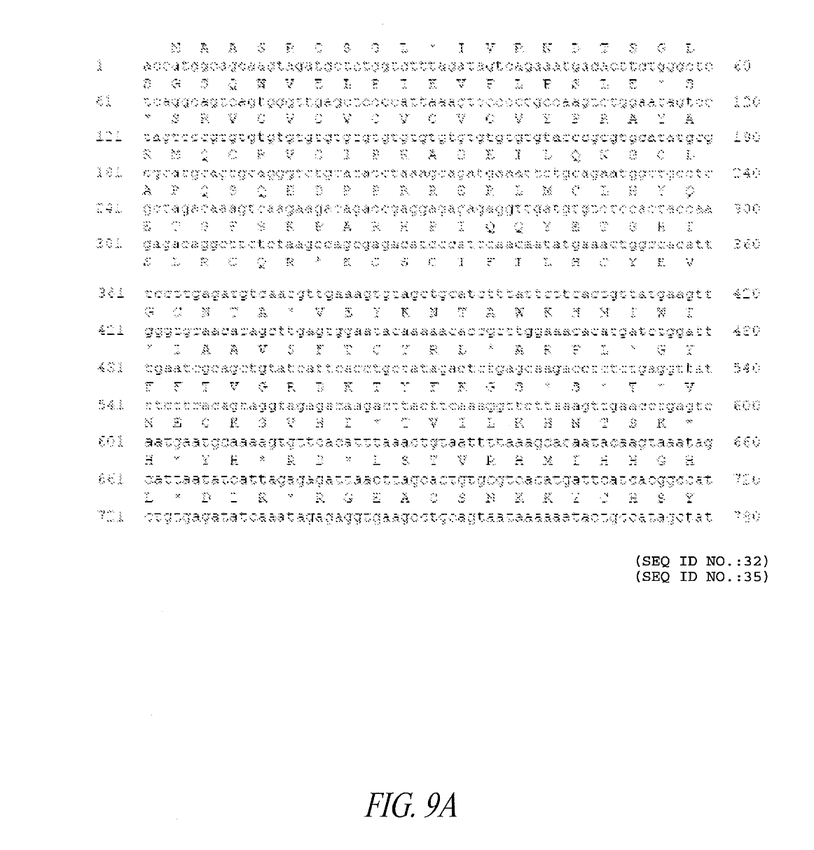

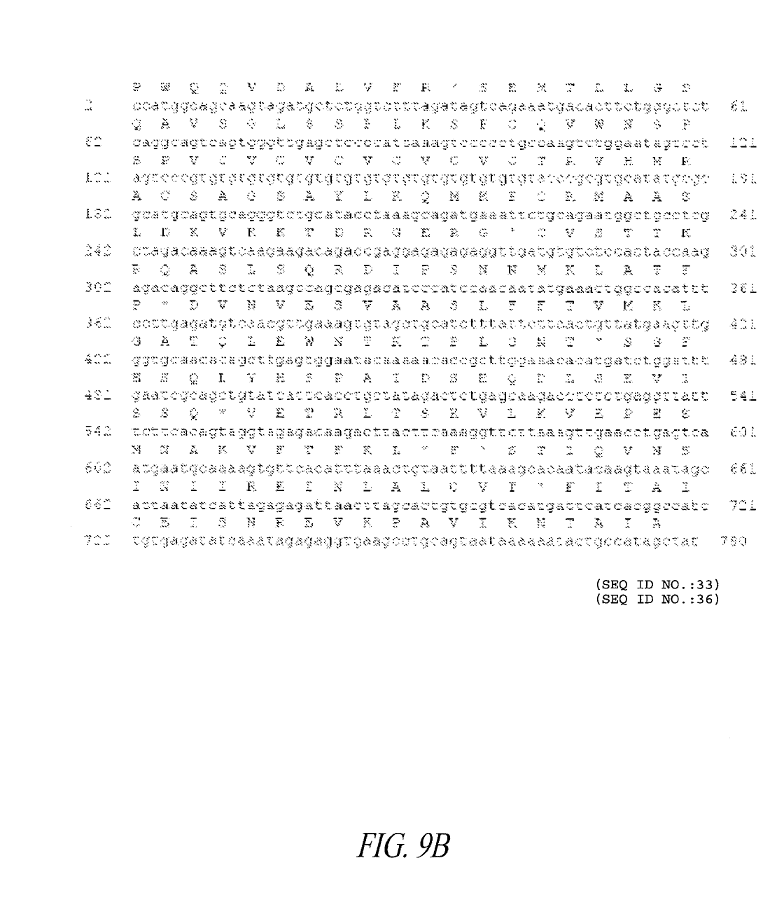

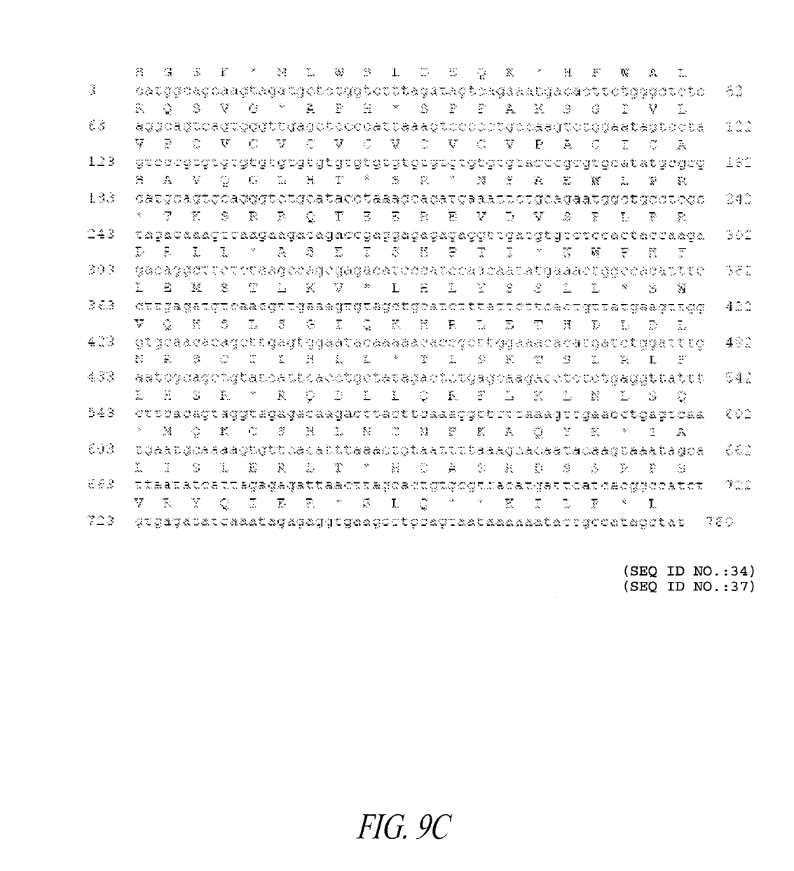

FIGS. 9A, 9B and 9C show, in view of the proposed shift −1 or +1 in reading frame at slippery or “shifty” sites (e.g., a shift of −1 or +1 in the reading frame after the ninth amino acid of Mig-7), three potential reading frames (−1, 0, +1) for the corresponding Mig-7 encoding sequences (SEQ ID NO: 35, 36 and 37). FIG. 9A shows the 0-frame, FIG. 9B shows the +1-shifted frame, and FIG. 9C shows the −1-shifted frame. According to aspects of the present invention, after the first nine Mig-7 amino acids are encoded in the 0 frame, subsequent coding may be in the +1 shifted frame, the −1 shifted frame, or in the 0 frame (e.g., in the case of reinitiation of translation), depending on the event at the first stop codon of the 0 frame. Moreover, regardless of the frame used after the ninth amino acid, corresponding stop codons are subsequently encountered in all three reading frames, and according to additional aspects of the present invention, encoding through these subsequent stop codons may involve recoding (e.g., for the +1 frame, three out of four of the subsequent stop codons are TGA stop codons that may encode for selenocysteine), read-through, or potentially re-initiation at these subsequent stop codon position.

Therefore, according to particular aspects, protein/polypeptides of the invention not only encompass a polypeptide according to SEQ ID NO: 32 and contiguous portions thereof, but additionally encompasses polypeptides comprising the first nine amino acids MAASRCSGL (SEQ ID NO: 31), followed by one or more frame-shifted amino acid sequence selected from SEQ ID NOS: 32, 33 and 34.

In preferred aspects, after the ninth amino acid, the +1-reading frame (from MAASRCSGL in the 0-frame to SEMTLL, RSEMTLL or MTLL in the +1-frame) is used, because this frameshift results in fewest downstream stops. This frame also contains a peptide that caused an increase breast carcinoma cell killing by peptide-stimulated monocytes from cancer patients in vitro, and overlapping peptides specific to this frame stimulate human peripheral blood monocyte killing of MCF-7 breast carcinoma cells in vitro in a statistically significant manner (see Example 1 herein). The TGA stop codon at position 532 can be read-through or frame shifted through, with the double-stop located between nucleotide positions 632 and 640 of SEQ ID NO: 35 being the likely ‘true stop’ given the detected 23 kD sized protein in immunoblots. However, because there are shift sites (slippery or “shifty” sites) at those stops as well, the protein may extend beyond the double-stop position shifting onto a different frame and end at a subsequent stop codon located between the double-stop and the polyA additional site beginning at nucleotide position 757 of SEQ ID NO: 35. All such proteins are encompassed herein.

Therefore, particular aspects of the invention provide a Mig-7 polypeptide frame-shift or genetically recoded polypeptide encoded by SEQ ID NO:1 and comprising an amino-terminal MAASRCSGL sequence. In particular aspects, the polypeptide is from about 203 to about 207 amino acid residues in length. In particular aspects, the polypeptide is from about 223 to about 240 amino acid residues in length.

In certain aspects, a frame-shifted or genetically recoded Mig-7 polypeptide encoded by SEQ ID NO: 1, comprises an amino terminal MAASRCSGLSEMTLLGSQAVSGLSSPLKSPC amino acid sequence (SEQ ID NO: 51), an amino terminal MAASRCSGLRSEMTLLGSQAVSGLSSPLKSPC amino acid sequence (SEQ ID NO: 52), or an amino terminal MAASRCSGLLDSQKMTLLGSQAVSGLSSPLKSPC amino acid sequence (SEQ ID NO: 53). In particular aspects, a Mig-7 polypeptide is from about 203 to about 207 amino acid residues in length. In particular aspects, the polypeptide is from about 223 to about 240 amino acid residues in length.

Preferably, the amino-terminal MAASRCSGL sequence is linked to at least one amino acid sequence selected from the group consisting of SEQ ID NOS:32, 33, 34, and contiguous portions thereof. In particular aspects, the polypeptide is from about 203 to about 207 amino acid residues in length. In particular aspects, the polypeptide is from about 223 to about 240 amino acid residues in length.

Preferably, the amino-terminal MAASRCSGL sequence is linked to at least one amino acid sequence selected from the group consisting of SEQ ID NOS:33, 34, and contiguous portions thereof. In particular aspects, the polypeptide is from about 203 to about 207 amino acid residues in length. In particular aspects, the polypeptide is from about 223 to about 240 amino acid residues in length.

Preferably, the amino-terminal MAASRCSGL sequence is linked to at least one amino acid sequence selected from the group consisting of SEQ ID NOS:33, and contiguous portions thereof. In particular aspects, the polypeptide is from about 203 to about 207 amino acid residues in length. In particular aspects, the polypeptide is from about 223 to about 240 amino acid residues in length.

V. The Polyamine, Spermine, Increased Translation of Mig-7

AZ frameshifting translation is enhanced by polyamines (Petros et al. Biochemical and Biophysical Research Communications 2005; 338, 1478-1489). In support of frameshifting and recoding events that lead to Mig-7 protein translation in vivo, the polyamine, spermine, enhanced synthesis of Mig-7 in HEC1A and in RL95 endometrial carcinoma cells (FIG. 8D). Interestingly, the lower band (˜22 kD non FLAG tagged) for Mig-7 protein was more prevalent in lysates that were not reduced prior to PAGE. In addition, the larger band was not initially detected in untreated HT29 cells expressing 3XFLAG Mig-7 under the CMV promoter in the absence of Matrigel (Crouch et al. Experimental Cell Research 2004; 292:274-87; and FIG. 6B). Whereas, a larger Mig-7 band (−50 kD) was detected in lysates from HT29 cells plated on the extracellular matrix Matrigel.

One explanation for this difference in Mig-7 protein size detected by immunoblot is that reduction and denaturing of highly cysteine-rich, >10% in the first 91 amino acids, Mig-7 was not complete causing the more compact non-reduced form to migrate further in the gel. This PAGE difference in size has also been determined for another cysteine-rich protein (Lucia-Ortega et al. Electrophoresis 2005; 26, 3407-3413). Indeed, the Applicant has determined that the peptide to the cysteine-valine dipeptide repeat region would not solubilize in vitro using various conditions (data not shown). Another explanation may involve multiple cysteine bond formation with other cysteine-rich proteins as previously mentioned. As a result, reducing Mig-7 cysteine interactions and keeping them reduced may cause the different sizes detected by immunoblot.

Notably, spermine allows read-through of UGA termination codons in another mammalian messenger RNA, rabbit beta-globin mRNA, when it is translated in a rabbit reticulocyte cell-free system. This read-through is specific to UGA and not UAA (Hryniewicz and Vonder Haar. Molecular and General Genetics 1983; 190, 336-343). Whereas in E. coli, read-through of the UAG stop codon, but not of UGA or UAA stop codons, is permitted with another polyamine, putrescine (Yoshida et al. J Biol Chem 2002; 277, 37139-37146). Furthermore, tumor cells overexpress the rate limiting enzyme, onithine decarboxylase (ODC), in polyamine biosynthesis and ODC is considered an oncogene (Gerner and Meyskens. Nat. Rev Cancer 2004; 4, 781-792). However, so far the use of difluoromethylornithine which irreversibly inhibits ODC, has had modest success as an anticancer therapy due to effective dose toxicity (Meyskens, Jr. and Gerner. Clin Cancer Res 1999; 5, 945-951). Moreover, cancer specific targets in this pathway have not previously been identified. Mig-7 may be such as target.

Polyamines also play an indirect role in protein modification. Specifically, polyamines increase casein kinase 2 (CK2) levels and activity (Childs et al. Cellular and Molecular Life Sciences 2003; 60, 1394-1406). Cancer cells overexpress CK2 due to their high levels of polyamines. CK2 has been suggested as a potential anti-cancer target (Ahmad et al. Anti-cancer Drugs 2006; 16, 1037-1043). However, unlike Mig-7, it is ubiquitously expressed and not cancer cell specific. Interestingly, Mig-7 protein, after the +1 frameshift, has six predicted CK2 phosphorylation sites (FIG. 8C). Thus, polyamines could be facilitating both translation of Mig-7, by enhancing frameshifting, and Mig-7 protein modification through phosphorylation by CK2.

FIG. 8D shows a immunoblots of lysates from HEC1A endometrial carcinoma cells cultured as previously described (Crouch et al. Experimental Cell Research 2004; 292:274-87) with the addition of 1.0 mM aminoguanidine and 0.1 mM spermine unless otherwise indicated. Aminoguanidine is required to prevent toxicity due to conversion of spermine to toxic byproducts by bovine calf serum oxidase (Sharmin et al. Biochemical and Biophysical Research Communications 2001; 282, 228-235). Immunoblots were performed as previously described except that affinity purified Mig-7 antibody (1:2,000) generated to the first 9 amino acids was used to detect endogenous Mig-7 produced by HEC1A and RL95 cells. Polyamine treatment induces ˜22 and ˜50 kD Mig-7 bands in HEC1A cells at 0.1 mM spermine. The results represent 2 independent experiments. FIG. 8E shows a representative immunoblot of lysates from RL95 cells treated with 1.0 mM aminoguanidine and 1.0 mM spermine (S) or no spermine (O). Both ˜22 and ˜50 kD Mig-7 bands were elevated in spermine treated cells. Equal amounts of protein (20 μg) were loaded in all experiments.

In summary, sequences that do not meet classical expression criteria are often not known or recognized in the art, as in the present case, and thus, are additionally not included on commercially available microarrays. Therefore, cancer researchers have heretofore been missing important Mig-7 gene expression data and compositions that provide insights and cell-specific reagents, therapeutics, and targets for cancer therapies, and markers of cancer, cancer progression/stage as well as recurrence. In this Example, novel Mig-7 nucleic acid and polypeptides sequences are provided.

Mig-7 sequence is reminiscent of a viral sequence in that it does not contain introns, it possesses viral-like repeat sequences, and genetic recoding likely allows translation. Because tumor microenvironment growth factors and interactions regulate Mig-7 expression which is sufficient for tumor cell invasion and vessel-like structure formation in 3-D cultures as well as its specificity to carcinoma cells and fetal trophoblasts (Crouch et al. Experimental Cell Research 2004; 292:274-87; Phillips and Lindsey. Oncology Reports 2005; 13:37-44), the described findings provide compositions and methods for broadly applicable diagnostic, prognostic and treatments of cancers.

### Example 3

### Expression of Mig-7 Allowed Cancer Cells to Sense a 3-D Environment, to Invade and to Form Vessel Structures

### Example Overview

Interactions between carcinoma cells and their environment are critical for disease progression. However, the molecular requirements are poorly understood. To address this problem, the Applicant utilized a novel human cancer-specific gene expression system, Mig-7, whose expression is restricted to cancer cells regardless of type.

Mig-7 is a cysteine-rich membrane protein whose mRNA and protein synthesis is not typical, as shown herein. Mig-7 expression is a result of receptor tyrosine kinase (RTK) c-Met activation as well as ligation of αvβ5. Mig-7 antisense constructs but not control sense strand constructs inhibit carcinoma cell scattering (Crouch et al. Experimental Cell Research 2004). Malignant tumors, blood from cancer patients and metastatic sites express Mig-7 regardless of tissue of origin. Notably, Mig-7 has not been detected in 25 different normal tissues (n=6 each tissue) or in blood from normal subjects (Crouch et al. Experimental Cell Research 2004; 292:274-87; Phillips and Lindsey. Oncology Reports 2005; 13:37-44).

Aggressive, invasive tumor cells can form vessel-like structures in 3-D cultures. Laminins-gamma2 promigratory fragments promote this vessel-like formation by aggressive melanoma cells in 3-D cultures. In vivo, predominantly tumor rather than endothelial cells form vessels in the interior, more hypoxic region of tumors (Hendrix et al. Ann NY Acad Sci 2003; 995:151-61). Moreover, RTK-induced cancer cell migration, invasion and dissemination of aggressive carcinoma cells require αvβ5 signaling, the crosstalk that induces Mig-7 (Crouch et al. Experimental Cell Research 2004; 292:274-87), in vivo and in vitro (Brooks et al. Journal of Clinical Investigation 1997; 99:1390-8; Klemke et al. Journal of Cell Biology 1994; 127:859-66). Fetal cytotrophoblasts are similar to cancer cells because they invade the maternal tissues during placenta development under RTK and αvβ5 signaling, evade immune system detection, endovascularly invade and are the only other cell type known to undergo vasculogenic mimicry (Folberg et al. Am J Pathol 2000; 156:361-81). Surprisingly, Mig-7 cDNA is homologous to ESTs isolated from early invasive stage placenta as well as all cancer types studied; while, in contrast, is not found in noninvasive term placenta or in other normal tissues (Crouch et al. Experimental Cell Research 2004; 292:274-87; Phillips and Lindsey. Oncology Reports 2005; 13:37-44).

Because carcinoma cells and cytotrophoblasts both engage in vasculogenic mimicry, in this Example, the Applicant tested and confirmed the hypothesis that Mig-7 is also expressed by fetal/embryonic cytotrophoblasts and plays a role in their common cell behaviors of invasion as well as vessel formation/restructuring resulting in leakiness. Importantly, 3-D cultures revealed that Mig-7 expression causes invasion and vessel formation. Adhesion assays to various components of the extracellular matrix suggests that a mechanism for Mig-7 in vessel formation by tumor cells is due to less adhesion to laminin. This Example provides further support for the efficacy of targeting Mig-7 in cancer therapies.

In studies using a nude mouse model of metastasis, Mig-7 protein primarily localized to vessels in lymph nodes to which subcutaneously injected carcinoma cells had spread. Tumor microenvironment growth factors, HGF, IGF-1 and EGF, each individually induced Mig-7 in αvβ5 integrin-positive carcinoma cells. αvβ5 integrin is important for tumor cell dissemination in vivo. Cytotrophoblasts, which possess αvβ5 integrin, expressed Mig-7 at a time point prior to their invasion on Matrigel. In 3-D cultures, expression of Mig-7 caused vessel structure formation. Lastly, Mig-7 expressing cells are >30% less adherent to a mix of laminins 1, 2, 3, 6, 8 & 10 in a statistically significant manner (p<0.001). Less adhesion to laminin is required for vasculogenic mimicry, a phenotype of aggressive cancer and fetal cytotrophoblast cells. The fact that Mig-7 is induced by growth factors that regulate PI3K, a signaling pathway required for vasculogenic mimicry, is localized to vessels in metastases, causes vessel-like formation and less adherence to laminin, collectively indicates that Mig-7 expression serves to allow cells to sense their environment, to invade, and to cause vasculogenic mimicry. Therefore, according to particular aspects of the present invention, Mig-7 provides a molecular target for therapies to modulate tumor progression.

### Methods and Materials:

Xenograft Mouse Model

The nude mouse model was performed as described previously (Phillips and Lindsey. Oncology Reports 2005; 13:37-44) under IACUC approval.

RNA isolation and Relative RT-PCR. RNA isolation and relative RT-PCR including optimization of cycle number to achieve mid-linear range were performed as described previously (1). PCR products were confirmed by Southern blot (Mig-7 specific cDNA probe) or by subcloning and sequencing.

Immunohistochemistry (IHC))

Detection of Mig-7 protein was performed using Mig-7 specific affinity purified antibody produced in rabbits immunized with conjugated Mig-7 peptide (MAASRCSGL) (SEQ ID NO: 31) representing the first nine amino acids of Mig-7 protein (Crouch et al. Experimental Cell Research 2004; 292:274-87). Briefly, antigen retrieval by microwave (2 seconds) was performed with cryostat sections (20 μm) of fresh frozen lymph node (three slides each) from nine endometrial carcinoma-injected and five control (Matrigel alone injected) nude mice. Cryosections were fixed immediately with 100% methanol at −70° C. for 10 minutes. Slides were washed 3× in PBS then pre-blocked in PBS containing 1% BSA, 5% preimmune rabbit serum, and 5% anti-mouse Fab2 fragment (SIGMA). Slides were then incubated simultaneously with anti-β-actin (1:1000, SIGMA), that recognizes both human and mouse β-actin, and Mig-7 antisera (1:50) overnight at 4° C. Slides were washed 3× in PBS containing 1% BSA then incubated for 30 minutes with antimouse IgG and antirabbit IgG both at 1:1000. After washing in PBS/BSA, slides were coverslipped with Vectashield Hard-set (Vector Labs) and analyzed using an Olympus IX-70 laser confocal scanning microscope equipped with an Olympus 60×/1.4 N. A. objective lens.

Western Blot Analyses

Western blot analyses were performed as previously described with the following modifications. Cells grown in (3-D) or on (2D) Matrigel cultures were homogenized in lysate buffer (2% SDS, 100 mM DTT, 0.01% bromophenol blue, 60 mM Tris, 10% glycerol, 2× protease inhibitor) and quantitated using RC/DC Protein Assay (Bio-Rad). Equal amounts of protein were loaded onto a 12% polyacrylamide gel and run at constant 200 V for 30-40 min. Gels were equilibrated in transfer buffer and semi-dry transferred (Boekel) for 1.5 h at 25 mAmp. Membranes were blocked in TBS-tween (0.05%) containing 5% dry milk for one hour at room temperature. Endogenous or FLAG-tagged Mig-7 protein was detected using affinity purified Mig-7 antibody (1:2,000) generated to the first 9 amino acids or the M2-peroxidase anti-FLAG antibody (1:100, Sigma), respectively. A HRP-labeled secondary anti-rabbit IgG antibody was used to detect the Mig-7 antibody at a dilution of 1:40,000. Chemiluminescence Plus Reagent (Amersham) allowed detection of HRP-labeled antibodies and exposed to film several times for different lengths of time.

Northern Blot Analyses

Northern blots were performed as previously described (Crouch et al. Experimental Cell Research 2004; 292:274-87).

Quantitative Real time RT-PCR

Reverse transcription of total RNA was carried out using the TaqMan Gold RT-PCR kit as described by the manufacturer (Applied Biosystems). This was followed by quantitative real-time PCR, which was performed using the Applied Biosystems 9700HT sequence detection system. Each target was amplified in triplicate with a Mig-7 specific primer and probe set or 18S Assay-on-Demand primer and probe set (Applied Biosystems). In triplicate, 5 μL of cDNA target was added to a 20 μL mix consisting of 1× TaqMan universal PCR master containing Amperase UNG and 1 μL of primer/probe. Reactions were incubated at 50° C. for 2 minutes, then 95° C. for 10 minutes. This was followed by 40 cycles of 95° C. for 15 seconds and 60° C. for 1 minute. A five-fold titration of control template was included in every run to assess PCR efficiency, along with a minimum of three negative controls. Levels of template were then calculated using the comparative Ct method using 18S as the endogenous control and time Oh as the calibrator (ABI User Bulletin #2). Data is presented as 2^{-ΔΔ}±SD.

Cell Cultures and Transfections

Human cytotrophoblasts were isolated from first or second trimester placentas under IRB approval and patient consent as previously described (Fisher et al. J Cell Biol 1989 Aug. 1; 109(2):891-902). Cytotrophoblasts were cultured on an extracellular matrix, Matrigel (BD Biosystems), at 37° C. in 5% CO2, which at 12 hours initiates their differentiation along the invasive pathway (Librach et al. J Cell Biol 1991 Apr. 1; 113(2):437-49). Cell cultures other than primary cytotrophoblasts were performed as previously described (Crouch et al. Experimental Cell Research 2004; 292:274-87; Phillips and Lindsey. Oncology Reports 2005; 13:37-44). HT29, HEC1A or RL95 cells were plated on Matrigel (1:6) dilution from confluent cultures for indicated times prior to collecting protein lysates. 3-D cultures were performed using 50 μl domes of Matrigel (no dilution) allowed to polymerize for 1 hour at room temperature, then 30 minutes at 37° C. in a humidified, 5% carbon dioxide incubator. Cells were removed from their plates non-enzymatically (2 mM EGTA) and 2 μl injected as single cell suspensions (7.5×10⁵) into the dome of Matrigel.

Statistical Analyses

All experiments were performed two or three times as indicated in figure legends. The one-way analysis of variance (ANOVA), Tukey-Kramer Multiple comparisons test and Grubb's test for outliers were used for statistical analyses in the GraphPad Prism statistical analyses software. P<0.05 was considered significant.

### Results:

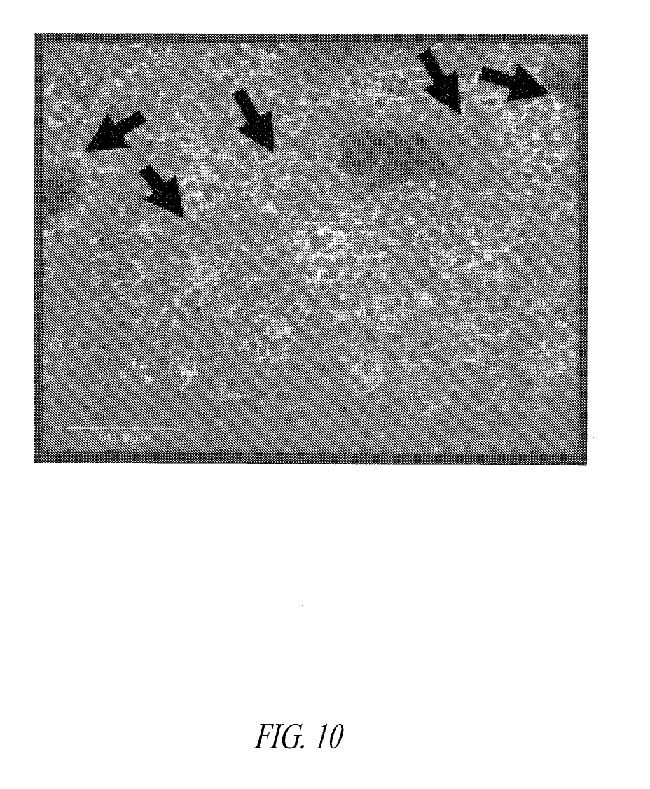

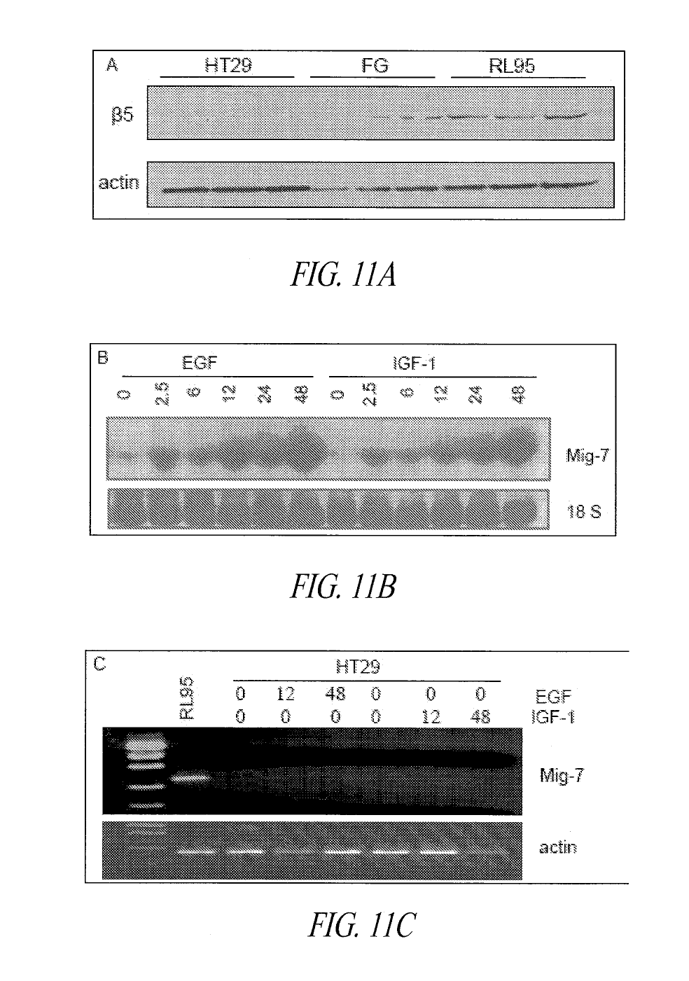

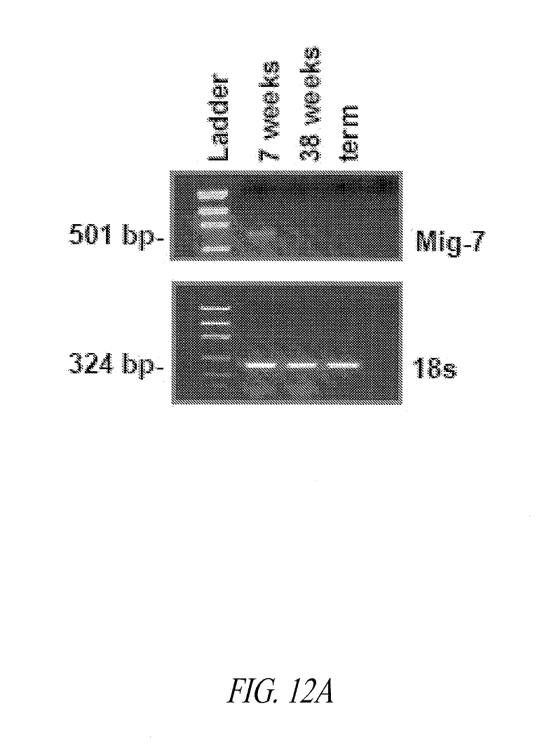

Mig-7 protein localized to vessel structures of metastases in a nude mouse model. The invasion capabilities of human endometrial carcinoma cell lines, RL95-2 (RL95) and HEC1A were tested in a xenograft nude mouse model, n=4 for each cell line. Mig-7 protein was localized in lymph nodes; cell localization was determined by fluorescent immunohistochemistry using a polyclonal antisera to the first 9 amino acids of Mig-7 protein and confocal analysis. Human specific Mig-7 predominantly localized to vessel structures as shown in FIG. 10 (arrows).

FIG. 10 shows that Mig-7 localizes primarily to vessels in xenograft nude mouse models of metastasis. Representative confocal analysis of Mig-7 protein immunohistochemical localization is shown. Rhodamine-conjugated anti-rabbit IgG (red) bound to Mig-7 antibody overlayed on the same area and sample scanned sequentially for Fluoroscein-conjugated anti-mouse IgG (green) binding to β-actin antibody. Arrows point to Mig-7 positive areas of vessels in lymph nodes to which subcutaneously injected HEC1A endometrial carcinoma cells in Matrigel had spread. Lymph nodes from animals injected with Matrigel alone (i.e., no cells) or incubated with antibody pretreated with Mig-7 peptide against which the antibody was generated were negative for Mig-7 (red) fluorescence. Detection of Mig-7 protein was performed using Mig-7 specific affinity purified antibody from antisera produced in rabbits immunized with conjugated Mig-7 peptide (MAASRCSGL) representing the first nine amino acids of Mig-7 protein (Crouch et al. Experimental Cell Research 2004; 292:274-87). Methods for immunohistochemistry are previously described (Phillips and Lindsey. Oncology Reports 2005; 13:37-44). Experiments were repeated twice with 4-5 animals per treatment group.

Tumor Microenvironment Growth Factors Mediated Expression of Mig-7 in αvβ5 Integrin Positive Cells

Mig-7 is detected in all types of cancers and it is known that other growth factors induce αvβ5 integrin activation. Tumor dissemination mediated by IGF-1 in vivo (Brooks et al. Journal of Clinical Investigation 1997; 99:1390-8) and in vitro, EGF-induced invasion of FG pancreatic carcinoma cells (Klemke et al. Journal of Cell Biology 1994; 127:859-66) requires this cross-talk signaling. Therefore, the Applicant tested whether other RTK ligands, IGF1 and EGF, can induce Mig-7 in αvβ5 integrin positive carcinoma cells. After obtaining FG cells (a gift from David Cheresh, Scripps Institute), the Applicant tested the expression of αvβ5 in these cells in comparison with two other carcinoma cell types, HT29 colon carcinoma cells and the cells from which we originally isolated Mig-7, RL-95 endometrial carcinoma cells.

Using a β5 antibody and Western blot analysis, HT29 cells lacked β5 whereas FG cells and RL95 cells were positive for this integrin subunit (FIG. 11A).

IGF-1 promotes tumor dissemination in vivo through cross-talk with αvβ5 (Brooks et al. Journal of Clinical Investigation 1997; 99:1390-8) In addition, the FG pancreatic carcinoma cell line requires signaling from αvβ5 in conjunction with EGF receptor activation for migration on vitronectin (Klemke et al. Journal of Cell Biology 1994; 127:859-66). Another RTK ligand, HGF induces Mig-7 in RL95 cells in an αvβ5-ligation dependent manner (Crouch et al. Experimental Cell Research 2004; 292:274-87). The Applicant used αvβ5-positive FG cells to examine if EGF and IGF-1. A time course after a single application of EGF or IGF-1 showed that both RTK ligands induce Mig-7 mRNA in FG cells (FIG. 11B). In addition, the levels of Mig-7 increase overtime with a single application of either ligand at time point 0 (FIG. 11B). In contrast, with αvβ5-negative HT29 cells (FIG. 11A), Mig-7 is not induced with either ligand even with 40 cycles of PCR after reverse transcription (FIG. 11C).

Specifically, FIGS. 11A-11C show, according to exemplary aspects of the present invention, that RTK ligands, IGF-1 and EGF, known to be produced in the tumor microenvironment, induce Mig-7 in αvβ5 integrin-positive carcinoma cells but not in αvβ5 negative HT29 cells. FIG. 11A shows a representative Western blot analysis of integrin β5 expression by HT29, FG and RL95 cells. HT29 are negative while FG and RL95 are positive for β5. FIG. 11B shows representative Northern blot analysis demonstrated that EGF and IGF-1 induce Mig-7 expression in β5+FG pancreatic carcinoma cells. RL95 cells express β5 and Mig-7 (Crouch et al. Experimental Cell Research 2004; 292:274-87). FIG. 11C shows that relative RT-PCR revealed that HT29 cells do not express Mig-7 mRNA. Each experiment was performed 3 times with similar results.

Cytotrophoblasts from Invasive Early Placenta Expressed Mig-7 Prior to and During Invasion.

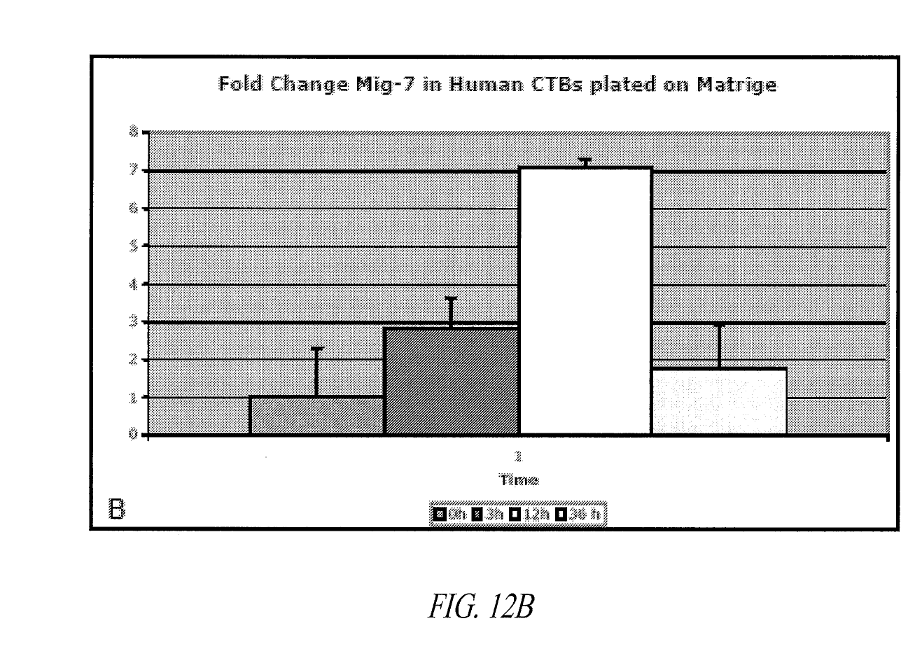

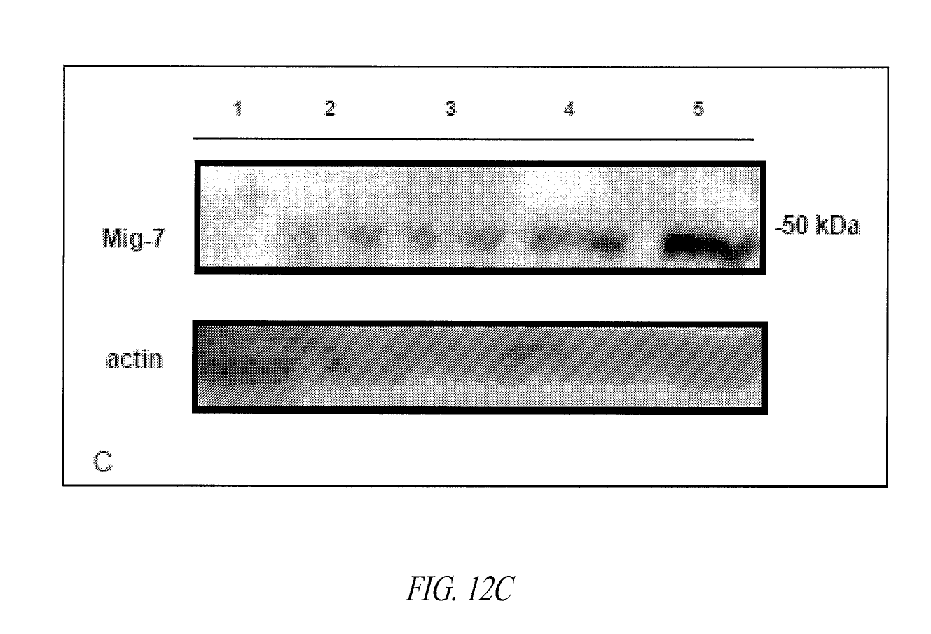

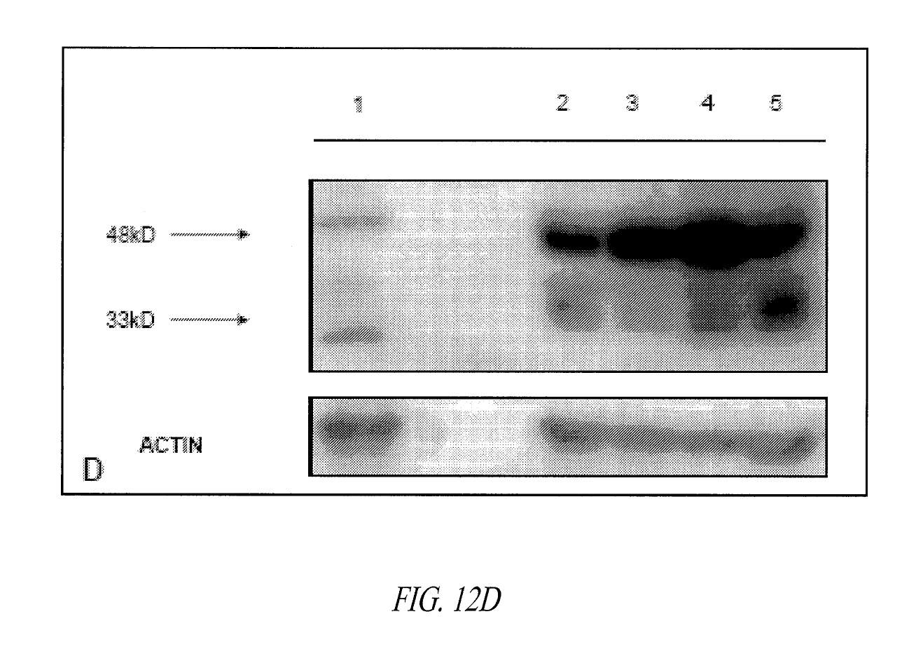

Mig-7 cDNA is homologous to ESTs from early placenta (Crouch et al. Experimental Cell Research 2004; 292:274-87). However, non-invasive term placentas lack Mig-7 (Crouch et al. Experimental Cell Research 2004; 292:274-87). Fetal cytotrophoblasts invade maternal deciduas and vasculature in early placental development prior to 22 weeks of gestation in humans behaving similar to cancer cells (Fisher et al. J Cell Biol 1989 Aug. 1; 109(2):891-902; Soundararajan and Rao. Reproductive Biology and Endocrinology 2004; 2(1):15). Basal plate placental tissues, the invasive front of embryonic/fetal cytotrophoblasts into maternal tissue, were isolated from early, late and term placentas. Relative RT-PCR was performed as previously described (Crouch et al. Experimental Cell Research 2004; 292:274-87; Phillips and Lindsey. Oncology Reports 2005; 13:37-44). Mig-7 mRNA was found in early placenta basal plate at 7 weeks of gestation but absent from late and term placentas (FIG. 12A).

Isolated cytotrophoblasts migrate on Matrigel beginning at 12 hours after plating (Librach et al. J Cell Biol 1991 Apr. 1; 113(2):437-49). Realtime PCR of Mig-7 mRNA expression revealed a 3-fold increase at 3 hours and at least a 7-fold statistically significant upregulation at 12 h after plating on Matrigel (FIG. 12B). Mig-7 protein levels also increased during a time course of primary fetal cytotrophoblasts plated on Matrigel. In 1^{st} trimester cytotrophoblasts, 12 hours Mig-7 protein level increases over 0 and 3 hours on Matrigel and continues to increase at 36 hours (FIG. 12C). In 2^{nd} trimester cytotrophoblasts, levels of Mig-7 are again highly induced prior to invasion on Matrigel (FIG. 12D).

Specifically, FIGS. 12A-D show that Mig-7 is expressed by invading, first and second trimester fetal cytotrophoblasts. FIG. 12A shows a relative RT-PCR analysis of Mig-7 mRNA expression in early and late fetal-maternal interface placenta. Mig-7 mRNA is found in early invasive placenta but not in late or term placenta. FIG. 12B shows a real-time RT-PCR analysis of cytotrophoblasts plated on Matrigel. Realtime PCR of Mig-7 mRNA expression revealed a 3-fold increase at 3 hours and at least a 7-fold statistically significant upregulation at 12 h consistent with initiation of cytotrophoblast invasion at this time on Matrigel. FIG. 12C shows a representative Western blot analysis of Mig-7 in 1st trimester cytotrophoblasts plated on Matrigel. 1—platelet lysate; 2—1st trim EVTs Oh; 3—1st trim EVTs 3 h; 4—1st trim EVTs 12 h; and 5—1st trim EVTs 36 h. D, Induction of Mig-7 protein expression in 2nd trimester cytotrophoblasts invading on Matrigel. 1—Platelet lysate; 2—2nd trim CTBs Oh; 3—2nd trim CTBs 3 h; 4—2nd trim CTBs 12 h; 5—2nd trim CTBs 36 h. CTB=cytotrophoblast.

Mig-7 Expression Caused Decreased Adhesion to Laminin and was Sufficient to Cause Vessel Formation in Matrigel 3-D Cultures.

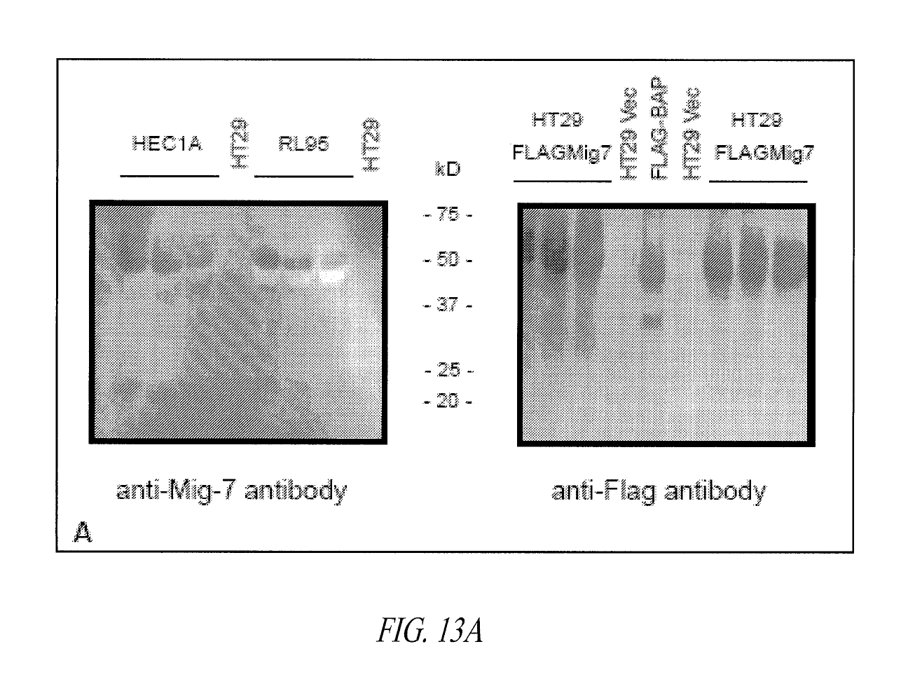

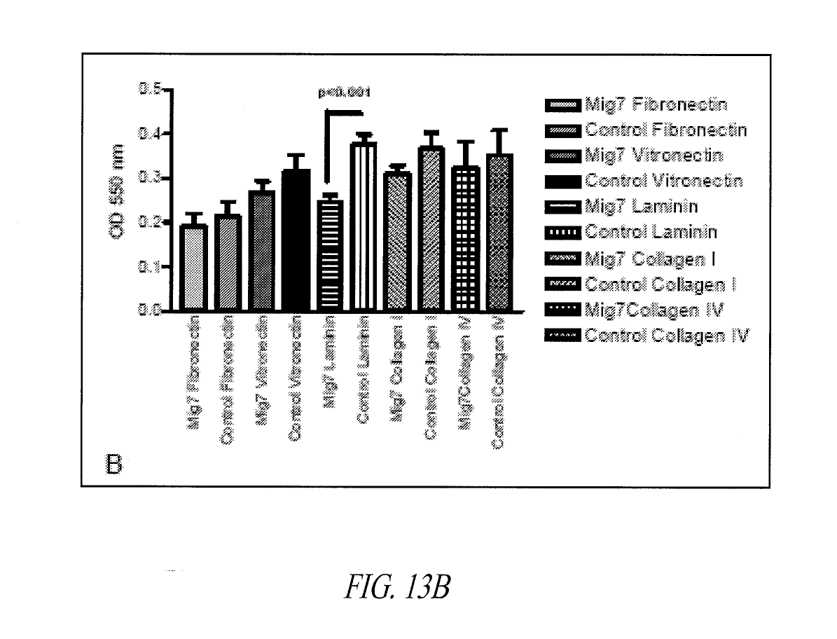

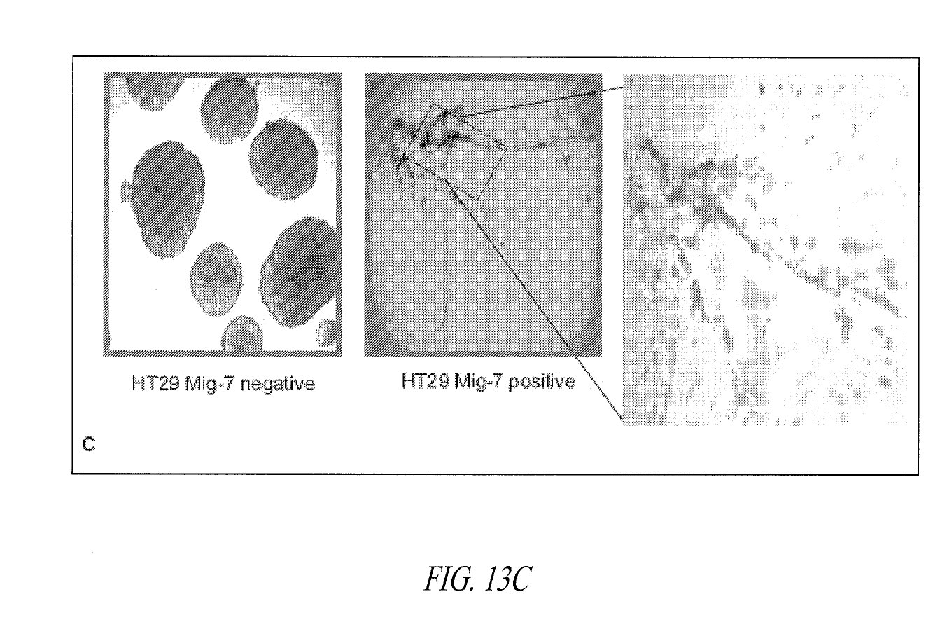

Because HT29 cells lacked αvβ5 expression (FIG. 11A) and RTK ligands did not induce Mig-7 in this cell line (FIG. 11C), the Applicant stably transfected HT29 cells with a Mig-7 expression vector as previously described (Crouch et al. Experimental Cell Research 2004; 292:274-87). FLAG-tagged Mig-7 produced by transfected HT29 cells was essentially the same size as endogenous Mig-7 expressed by either HEC1A or RL95 endometrial carcinoma cell lines (FIG. 13A).

After stable transfections, that Mig-7 expressing cells were observed to be less adherent to culture plastic. To determine if this less adherent phenotype translated to components of the extracellular matrix, adhesion assays were performed and quantified as described in Methods and Materials. Mig-7 expressing HT29 cells were significantly less adherent to laminin coated wells. Whereas, adhesion to other ECM components, fibronectin, vitronectin, collagen I or collagen IV were unaffected by Mig-7 expression (FIG. 13B).

Modification of laminins have been shown to be important for cancer cell invasion (Givant-Horwitz et al. Cancer Letters 2005; 223:1-10) and for vasculogenic mimicry (Seftor et al. Cancer Res 2001; 61:6322-7). Mig-7 is important for cell migration in vitro and found in blood from metastatic cancer patients, primary tumors and metastatic site tumors (Crouch et al. Experimental Cell Research 2004; 292:274-87; Phillips and Lindsey. Oncology Reports 2005; 13:37-44). Applicant had not detected any phenotypic changes in HT29 Mig-7 expressing compared to nonexpressing cells in 2D cultures. Applicant used Matrigel 3-D cultures which are similar to the tumor microenvironment more than 2D cultures. HT29 parental or empty vector transfected cells formed discrete colonies in 3-D cultures in stark contrast to Mig-7 expressing HT29 cells that invaded and formed vessel structures (FIG. 13C).

Specifically, FIGS. 13A-C show that Mig-7 expression causes decreased adhesion to laminin and was sufficient to cause vessel formation in Matrigel 3-D cultures. FIG. 13 A shows that HT29 cells transfected with 3XCMVFLAG-Mig-7 expression vector express the same sized protein detected by anti-FLAG antibody as does the affinity purified Mig-7 antibody of endogenous Mig-7 in HEC1A cell lysates. Cells were plated on Matrigel for 17, 48 and 72 hours. Empty vector transfected HT29 cells are negative for FLAG Mig-7 as are parental HT29. FIG. 13B shows that HT29 Mig-7 expressing cells are >30% less adherent to a mix of laminins 1, 2, 3, 6, 8 & 10 in a statistically significant manner (p<0.001). FIG. 13C shows that HT29 cells with empty vector form discrete colonies in 3-D Matrigel cultures (left panel). In contrast, HT29 cells expressing FLAG Mig-7 invade and form vessel structures (right panel and inset, supplemental data). All experiments were performed at least twice (3-D cultures three times) in quadruplicate.

Tumor microenvironment growth factors, HGF, EGF or IGF-1 induce carcinoma-specific Mig-7 in αvβ5 integrin-positive cells. This redundancy suggests that the expression of Mig-7 is important for the invasive capabilities of tumor cells. αvβ5 integrin ligation is required for cytotrophoblast invasion (Zhou et al. Journal of Clinical Investigation 1997; 99(9):2139-51) and for tumor dissemination in vivo (Brooks et al. Journal of Clinical Investigation 1997; 99:1390-8; Klemke et al. Journal of Cell Biology 1994; 127(3):859-66). It is well known that these growth factors promote tumor progression and metastasis. Results suggest that multiple growth factors of the tumor microenvironment induce Mig-7 which accounts for its expression in virtually all cancer samples (n>240) irrespective of tissue of origin (Crouch et al. Experimental Cell Research 2004; 292:274-87; Phillips and Lindsey. Oncology Reports 2005; 13:37-44). When cancer cell lines are plated on Matrigel, which contains all of these growth factors, Mig-7 protein levels are elevated. Inclusion of Mig-7 sequences on microarrays will help further define its regulation at the mRNA level.

Early placenta, which is the invasive stage of placenta development, expresses Mig-7 mRNA consistent with previous data of ESTs from early placenta are homologous with Mig-7 cDNA (Crouch et al. Experimental Cell Research 2004; 292:274-87). Fetal cytotrophoblasts from placenta prior to 22 weeks of gestation mimic behavior of invasive tumor cells (Folberg et al. American Journal of Pathology 2000; 156(2):361-81). Fetal cytotrophoblasts express Mig-7 mRNA and protein when plated on Matrigel for 12 hours at seven fold higher levels than those initially plated. This is the time period in which cytotrophoblasts start invasion on Matrigel (Janatpour et al. Development 2000 Feb. 1; 127(3):549-58) suggesting Mig-7 may play a role in their invasion. Fetal cytotrophoblasts from second trimester express Mig-7 consistent with a potential role for Mig-7 in endovascular invasion by these cells during the second trimester (Zhou et al. Journal of Clinical Investigation 1997; 99(9):2139-51). Aggressive tumor cells also endovascularly invade in order to metastasize. Interestingly, EGF acts as a chemoattractant for intravasation of blood vessels by tumor cells (Condeelis et al. Annual Review of Cell and Developmental Biology 2005; 21(1):695-718).

Mig-7 causes significantly lower adhesion to culture plastic and to laminins (FIG. 13B). Laminin is a major component of Matrigel and the basement membrane of epithelial structures and has been shown to play an important role in tumor cell behaviors (Givant-Horwitz et al. Cancer Letters 2005; 223:1-10). Therefore, because Mig-7 makes cells less adhesive to laminin this may facilitate tumor cell invasion. By bringing important mediators together at the cell membrane through one or more of its cysteine residues, Mig-7 may play also play a role in production of Laminin 5 gamma 2 promigratory fragments which would make cells less adherent to laminins.

Notably, Mig-7 expression in HT29 cells is sufficient and necessary for vessel formation in 3-D cultures (FIG. 13C) suggesting that Mig-7 allows sensing of the microenvironment. Localization of Mig-7 protein to cells of vessel structures in lymph nodes to which human carcinoma cells spread in the nude mouse model of metastasis implies that either these cells are forming vessels due to their Mig-7 expression or that these cells are intravasating or extravasating. No difference in phenotype, other than a lack of adherence, is observed in 2D cultures on plastic or Matrigel (See Example 1 and 3 herein). Plasticity of tumor cells that form vessels has been termed vasculogenic mimicry and predicts poor patient outcome. The formation of laminin 5 gamma 2 promigratory fragments and the presence of EphA2 are important for the process of vasculogenic mimicry (Hendrix et al. Ann NY Acad Sci 2003; 995:151-61). Cysteine rich Laminin 5 gamma 2 fragments have also been shown to be important in other epithelial invasion as well as migration and is not restricted to aggressive cancer cells (Givant-Horwitz et al. Cancer Letters 2005; 223:1-10). EphA2, another cysteine-rich protein, is localized at the membrane and phosphorylated differentially between normal and malignant cells. Importantly, a different phenotype is also observed with transformed cells overexpressing EphA2 in 3-D cultures as compared to 2D cultures. EphA2 is also found on chromosome 1 as is Mig-7 in a “hot spot” for cancer (Walker-Daniels et al. American Journal of Pathology 2003; 162(4):1037-42). Because EphA2 or Mig7 expression imparts dramatically different phenotypes in 3-D cultures, it is tempting to speculate that they may be associated.

It has been proposed that finding a protein specific to vasculogenic mimicry would be an excellent anti-cancer target because this process does not occur in any normal tissue in children or adults. Additionally, invading cancer cells are resistant to cancer therapies targeting cell growth (Condeelis et al. Annual Review of Cell and Developmental Biology 2005; 21(1):695-718). Therefore, therapies directed specifically to invading cancer cells are needed. According to particular aspects, Mig-7 provides a carcinoma-, invasion-specific target to inhibit invasion and vessel formation in vivo preventing dissemination of tumor cells and death. The facts that Mig-7 is induced by growth factors that regulate PI3K, a signaling pathway required for vasculogenic mimicry (Hess et al., Cancer Res 2003 Aug. 15; 63(16):4757-62), is localized to vessels in metastases, causes vessel-like formation as well as invasion, collectively indicatees that Mig-7, at least in part, controls invasion and vasculogenic mimicry in tumor cells.

### Example 4

### Stable Knockdown of Mig-7 Expression Reduces Tumor Growth In Vivo

### Experimental Overview and Methods

This example shows that down-regulation of Mig-7 expression in carcinoma cells leads to a reduction in overall tumor growth.

In vitro expression of Mig-7 specific siRNA from construct 1-3A (Table 5) inhibited RL95 cell invasion in 3-D cultures, and showed a 60-70% decrease in Mig-7 protein levels in RL95 cells expressing Mig-7 siRNA 1-3A.

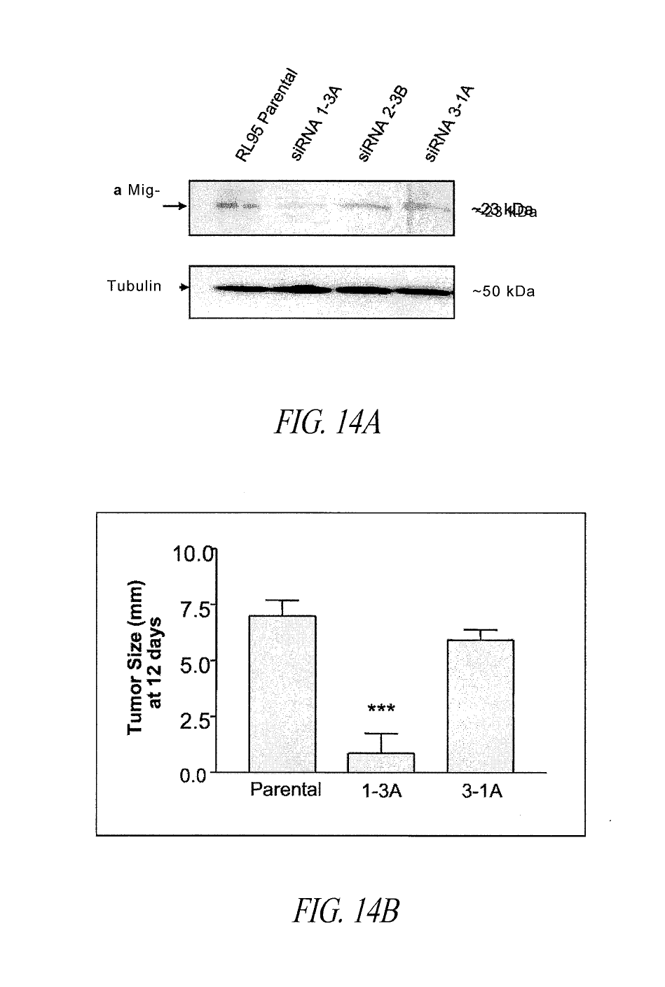

Stable knockdown of Mig-7 through siRNA expression, was studied on primary tumor growth in vivo. Two RL95 endometrial carcinoma cell lines stably transfected with different siRNA expression plasmid constructs, 1-3A and 3-1A (Table 5), were injected into nude mice and measured tumor growth compared to parental RL95 injection. Mice injected with RL95 cells expressing siRNA 1-3A showed a significant decrease (70%) (p<0.001) in linear tumor size 12 days after injection compared to mice injected with parental RL95 cells. In contrast, mice injected with RL95 cells expressing siRNA 3-1A that express endogenous levels of Mig-7 showed no significant decrease in primary tumor size (FIG. 14B). Reduction in tumor growth observed may be due to inhibition of vessel formation by tumor cells, inhibition of tumor cell metastasis, and/or inhibition of tumor cell proliferation. FIG. 14A shows a reduction in Mig-7 protein levels in samples expressing siRNA (1-3A or 3-1A) compared to control (parental) cells.

TABLE 5
siRNA Sequence Constructs Specific to Mig-7
Sequence anti-sense-loop-sense

| | |
|---|---|
| 1. 5′-AAGTTTCATTCTTCGACTTCAA | by 379 to 398, |
| GAGAGTCGAAGAAATGAAACTTT-3′ | clones number 1-3A; |
| | SEQ ID NO: 43 |
| 2. 5′-GATTTCCTGTGATTTAAGTTCAA | by 728 to 746, |
| GAGACTTAAATCACAGGAAATCT-3′ | clones number 2-3B; |
| | SEQ ID NO: 44 |
| 3. 5′-ATGATCTGGATTTGAATCTTCAA | by 1275 to 1293, |
| GAGAGATTCAAATCCAGATCATG-3′ | clones number 3-1A; |
| | SEQ ID NO: 45 |

The present invention also predicts that expression of variants of Mig-7 specific siRNAs will inhibit RL95 cell invasion in 3-D cultures. This prediction is based, in part, on the observation that the siRNA duplexes function well with 1- or 2-bp overhangs, and may, in some cases, have improved efficiency of siRNA methods (Sano et al., 2008; Miller et al., 2008). In addition, those of ordinary skill in the art will appreciate that imperfect hybridization between target and siRNA is well tolerated (reviewed in Storz et al., 2005).

The siRNAs encoded by the siRNA constructs represented in Table 5 and by SEQ ID NOs: 43-45 are set forth in SEQ ID NOs: 54-56.

### Example 5

### Targeting Mig-7 Inhibits Carcinoma Cell Invasion, Early Primary Tumor Growth, and Stimulates Killing of Breast Carcinoma Cells

### Example Overview

Discovery and targeting of tumor cell-specific gene expression could lead to more effective cancer treatments with less toxic side effects. Furthermore, targeting tumor cell proteins that facilitate their invasion, during which tumor cells are resistant to many current therapies, could provide additional efficacy and less recurrence of disease.

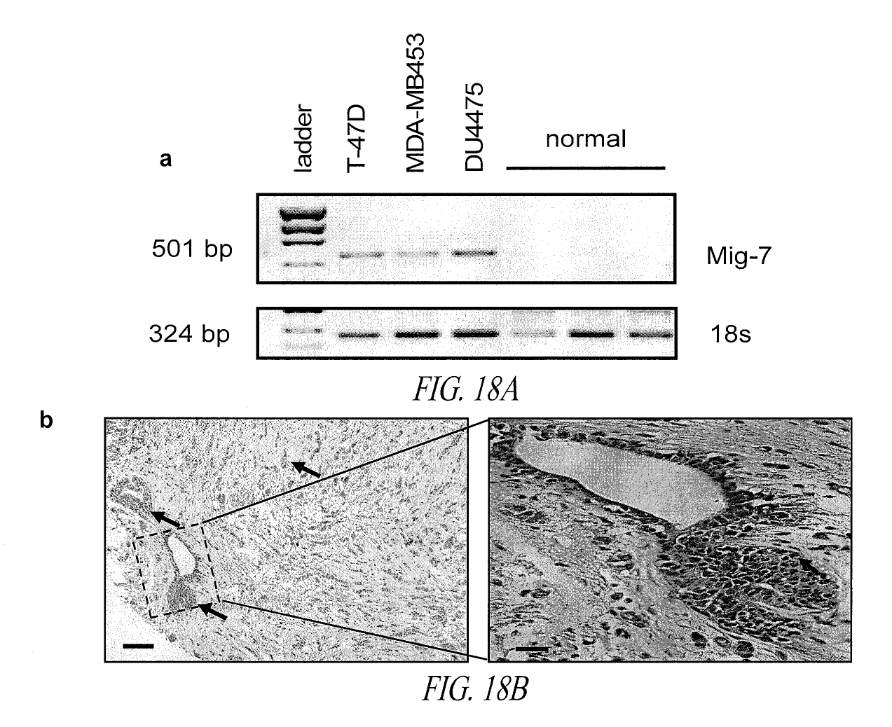

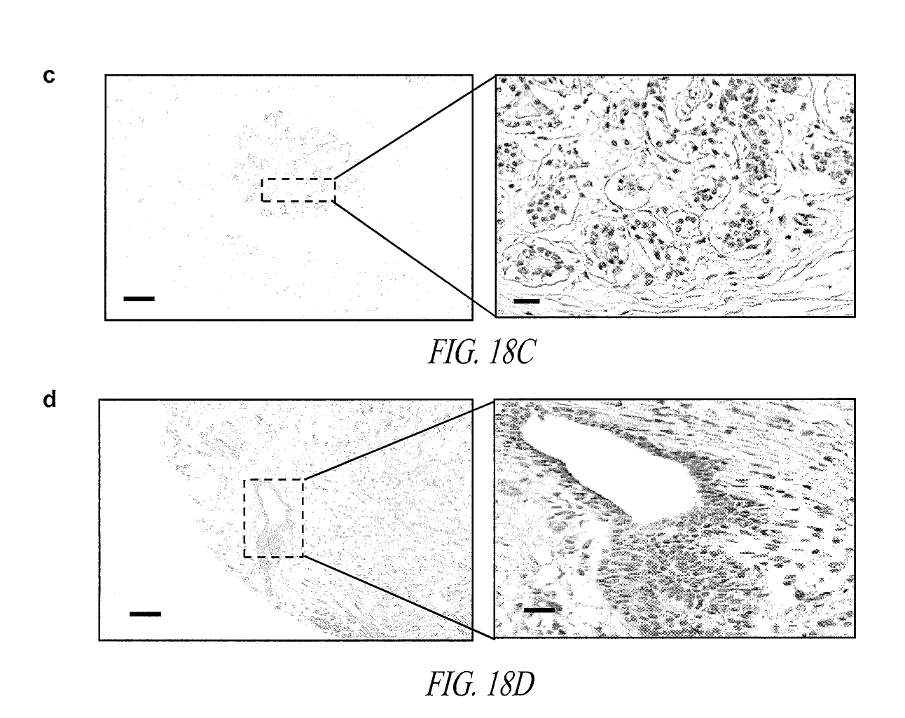

One such human protein, Mig-7, is expressed by tumor cells in circulation, in primary and in metastatic tumor sites as well as by occult tumor cells that mimic endothelial cells. Multiple tumor microenvironment factors, such as epidermal and hepatocyte growth factors, induce Mig-7 mRNA expression. Gain or loss of Mig-7 protein studies showed that its expression leads to colon and endometrial carcinoma cell invasion. Further, Mig-7 expression was detected in tumor cells of breast carcinoma and a subset of precancerous breast samples but not in cells from normal breast tissue samples (see FIG. 18). Thus, targeting Mig-7 would decrease tumor cell invasion, enhance monocyte cell killing of tumor cells and inhibit disease progression.

To test this hypothesis an in vitro analysis of peptide-stimulated human peripheral blood monocyte cells (MC) and their killing of MCF-7 breast carcinoma cells was used. Mig-7 peptide treatment increased MC tumor necrosis factor expression and killing of MCF-7 cells >2-fold over MUC-1 or control peptide treatments.

In another approach to test this hypothesis, an in vitro chemoinvasion assay of endometrial carcinoma cells treated with Mig-7 specific or control antibodies was used. Mig-7 antibody significantly reduced invasion by >60% compared to controls. Targeting Mig-7 with an antibody to its first nine amino acids reproducibly and significantly inhibited endometrial carcinoma cell invasion in vitro.

Furthermore, stably expressing Mig-7-specific short interfering RNA resulted in significantly reduced Mig-7 protein levels and early primary tumor growth in a xenograft nude mouse model. In vivo studies showed that decreased Mig-7 expression significantly impaired early tumor growth in an endometrial cell xenograft nude mouse model. Reduced phosphorylation of ERK1/2, Akt, and S6 kinase as well as decreased membrane-type 1 matrix metalloproteinase activity were mechanisms through which Mig-7 protein caused these effects. Based on these collective data, Mig-7 expression is a potential candidate for future targeted cancer therapies. Active states of membrane-bound metalloproteinase MT1-MMP (also known as MMP-14), ERK1/2, Akt, and S6 kinase were all reduced with Mig-7 targeting.

### Materials and Methods

Cell Cultures

Methods for transfection and cultures of HEC1A, RL95 endometrial carcinoma parental (Crouch et al. Experimental Cell Research 2004; 292:274-87) and MCF-7 breast carcinoma (Wright et al. Journal of Immunotherapy 2000; 23:2-10) cells were previously described. Under Institutional Review Board approval, human isolated monocyte cells (MC) were isolated and cultured at 2×10⁶ cells/ml in AIM-V^{R} serum-free lymphocyte medium (Gibco, Invitrogen, Carlsbad, Calif.) as previously described (Wright et al. Journal of Immunotherapy 2000; 23:2-10). Experiments in Example 5 were conducted with the following siRNA constructs, listed in Table 6. The siRNAs encoded by the siRNA constructs represented in Table 6 and by SEQ ID NOs: 46-49 are set forth in SEQ ID NOs: 57-60.

TABLE 6

| | |
|---|---|
| 1. 5′-AAAAGTTTCATTTCTTCGACTTCAAGAGA | bp 379 to 398 of |
| GTCGAAGAAATGAAACTTTT-3′ | DQ080207, |
| | clones number |
| | 1-3; |
| | SEQ ID NO: 46 |
| 2. 5′-AGATTTCCTGTGATTTAAGTTCAAGAGA | bp 728 to 746 of |
| CTTAAATCACAGGAAATCT-3′ | DQ080207, |
| | clones number |
| | 2-3; |
| | SEQ ID NO: 47 |
| 3. 5′-CATGATCTGGATTTGAATCTTCAAGAGA | bp 1275 to 1293 |
| GATTCAAATCCAGATCATG-3′ | of DQ080207, |
| | clones number |
| | 3-1; |
| | SEQ ID NO: 48 |
| 4. 5′-TCATTCACCTGCTATAGACTTCAAGAGA | bp 1303 to 1321 |
| GTCTATAGCAGGTGAATGA-3′ | of DQ080207, |
| | clones number |
| | 4-2; |
| | SEQ ID NO: 49 |

Modified Boyden Chamber Invasion Assay

Chemoinvasion assays were performed as previously described (Hendrix et al. Invasion & Metastasis 1989; 9:278-97). Briefly, transwell filters (Costar, Corning, N.Y., 8 μm) were blocked in 1% BSA-DMEM/F12 for 30 minutes and rinsed with phosphate-buffered saline (PBS). Matrigel (BD Biosciences, San Jose, Calif.) was diluted in ice cold PBS to 1000 μg/mL to coat the lower side of each pre-chilled transwell filter insert. After incubating the coated inserts at 37° C. for one hour and washing with PBS containing Ca²⁺ and Mg²⁺, cells were detached using trypsin without EDTA, neutralized with soybean trypsin inhibitor, centrifuged for 5 minutes at 1000 rpm (4° C.), and washed one time in DMEM/F12. Cell count and viability were determined using trypan blue exclusion and a hemacytometer. Cells were preincubated with 10 μg/mL affinity-purified Mig-7 rabbit polyclonal antibody (Petty et al. Am J Pathol 2007; 170:1763-80; Phillips and Lindsey. Oncology Reports 2005; 13:37-44) or control normal rabbit IgG antibody for 15 minutes in a 37° C. incubator. Rabbit IgG antibody served as control, as previously described (Yang et al. Cancer Res 2006; 66:46-51). Media containing the chemoattractant, hepatocyte growth factor (HGF, R&D Systems Inc., Minneapolis, Minn.), was added to bottom wells at a concentration of 20 ng/mL. Media without HGF containing 50,000 cells was added to each top well. Cells were allowed to invade for 72 hours at 37° C. in 5% CO₂, 95% air humidified incubator. After invasion, filters were rinsed with PBS then fixed in Hema3 fixative (Fisher Scientific, Inc., Pittsburgh, Pa.) for at least 30 minutes. Non-invaded cells in the upper chamber were removed with a cotton swab. Filters were dried and stained with Hema3 (Fisher Diagnostics, Middletown, Va.) according to manufacturer's instructions. Filters were mounted on slides with gridded coverslips to count invaded cells using a microscope (Electron Microscopy Science, Hatfield, Pa.) at 400× magnification with a count of 10 squares (0.6×0.6 mm each) per filter from each treatment. Percent invasion was calculated by extrapolating the average cell count for the entire filter surface area and dividing by initial cell number. No cells were observed in bottom wells that could have invaded through the Matrigel. All treatments were performed in triplicate and experiments were repeated three times.

Apoptosis Assay

Apoptosis was assessed using the Vybrant Apoptosis Assay Kit #4 (Molecular Probes, Eugene, Oreg.). For RL95 siRNA expressing cell lines, 1×10⁶ cells were plated in triplicate on 6-well ultra-low attachment plates (Corning, Inc., Corning, N.Y.) for 18 hours prior to analysis. For HEC1A cells, 1×10⁶ cells were plated in 24-well plates and treated with Mig-7 antibody or rabbit IgG (10 μg/ml each) for 72 hours prior to analysis. Cells were trypsinized, pelleted by centrifugation, then washed once in 1×PBS. After resuspending the cells in 250 μl of PBS, the cells were transferred to 96-well plates. 1 μl of a 1:4 dilution of both YO-PRO-1 (100 μM) and propidium iodide (PI) (1 mg/ml) was added to cells and incubated on ice for 30 min. Cells left unstained, or stained with YO-PRO-1 or PI alone, were also included for controls. Listmode data were collected using a FACSCalibur (Becton Dickinson, Bedford, Mass.) flow cytometer and Winlist analysis software (Verity Software, Topsham, Me.). Single-stained samples were used to perform compensation. Cells were gated to exclude large cell clumps and very small debris. YO-PRO-1 positive staining indicated apoptotic cells. YO-PRO-1 and PI double staining indicated dead cells. All cell lines or treatments were analyzed in triplicate and the experiments were performed twice with similar results.

Proliferation Assay

Cell proliferation was assessed by using PI nuclear staining dye and flow cytometry as previously described (Noguchi. Current Protocols in Immunology. New York: Green Publishing Associates and Wiley-Interscience; 1991. p. 5.7.1-5.7.4). HEC1A cells were counted and 5×10⁵ cells were plated in a 24-well plate and treated with IgG or Mig-7 antibodies as described elsewhere herein. For RL95 siRNA expressing cell lines, 5×10⁵ cells were plated and grown for two days prior to collection. All cells were fixed in 1 ml of 70% ethanol overnight. After fixation, cells were centrifuged (470×g, 5 min., 4° C.) and the pellets washed once in 1× staining buffer (Dulbecco's PBS containing 2% FBS and 0.01% NaN₃). Cells were then treated with 1 mg/ml RNase A (Sigma, St. Louis, Mo.) in PBS at 37° C. for 30 min. After removing the RNase A solution, 300 μl of staining buffer containing 20 μg PI was added to resuspended cells and incubated 30 min. at room temperature. PI was not added to one sample as a negative control. After 30 min., PI staining was analyzed using the FACSCalibur (Becton Dickinson, Bedford, Mass.). Levels of PI staining correlated to the different cell cycle phases, and the numbers of cells in the G2/M phase (highest PI expression) were compared as an indicator of proliferation. Cells were gated to exclude large cell clumps and small debris. All cell lines or treatments were analyzed in triplicate and the experiments were performed twice with similar results.

α2-Macroglobulin Capture Assay

This assay was performed as previously described (Windsor et al. J Biol Chem 1994; 269: 26201-7). Briefly, cell lines were plated at confluency and treated as indicated in a 6-well plate on 1 mg/ml Matrigel for 18-20 hours before the assay. Cells were then removed by scraping and each sample was split into two wells of a 24-well plate. Purified human α2-macroglobulin (MP Biomedicals, Solon, Ohio) was added to one of the two wells for each sample at a concentration of 1 mg/ml and incubated at room temperature for 15 min. Cells without α2-macroglobulin served as control. Following incubation, lysis buffer [2% SDS, 60 mM Tris pH 6.8, 10% glycerol and 2× protease inhibitors (Complete, Roche, Indianapolis, Ind.)] was added, at a 1:2 final dilution, and immunoblots performed as detailed elsewhere herein. Assays were performed in triplicate for each cell line and treatment. Cells without α2-macroglobulin were pooled for immunoblot analysis. Individual background levels for each lane were subtracted from the band of interest level then divided by respective tubulin level for densitometry calculations.

Protein Phosphorylation Analyses

For protein phosphorylation analyses, confluent RL95 siRNA-expressing cells were plated on 1:10 diluted Matrigel (>12 mg/ml) for 19 hours prior to harvest. Confluent cells were trypinized, pelleted at 4° C. at 1000 rpm for 4 min. and washed three times in media, followed by three washes in cold TBS. Multiprotein phosphorylation analysis using antibodies and Luminex Bead-based immunoassays was performed by AssayGate, Inc. (Ijamsville, Md.). Briefly, indicated proteins in each cell lysate were determined quantitatively and simultaneously with Bio-Plex 200 Bead Reader System. Antibodies were labeled with differing concentrations of two fluorophores to generate distinct bead sets. Each bead set was coated with capture antibody specific for each indicated total or phospho-protein. Captured analyte was detected using a biotinylated detection antibody and streptavidin-phycoerythrin (S-PE). Analyses were performed using a dual laser, flow-based, sorting and detection platform. One laser was bead-specific and determined a given antibody bound to protein. The other laser determined the magnitude of PE-derived signal, which is in direct proportion to the amount of protein bound. Protein concentrations of samples were determined by 5-parameter logistic regression algorithm with analysis of the median fluorescence intensity readings of an 8-point protein standard curve. Once a regression equation was derived, the fluorescence intensity values of the standards were treated as unknowns and the concentration of each standard was calculated. A ratio of the calculated value to the expected value of this standard was determined. A ratio between 70 and 130% for each of the standards indicated a good fit. Precision was evaluated by the coefficient of variation which equals the standard deviation divided by the mean. All samples indicated a good level of precision.

Antibodies to phosphorylation sites analyzed included Ser⁴⁷³ for Akt, Thr²⁰²/Tyr²⁰⁴ and Thr¹⁸⁵/Tyr¹⁸⁷ for extracellular signal-regulated kinase 1/2 (ERK1/2), Thr²⁴⁶ for proline-rich Akt substrate of 40 kDa (PRAS40), Thr⁴²¹/Ser⁴²⁴ for S6 kinase, and Tyr¹¹³⁵/Tyr¹¹³⁶ for insulin-like growth factor-1 receptor (IGF-1 R). Antibodies to each protein were also used to detect total protein levels. Samples were tested in triplicate.

Immunoblotting

Cell lysates and immunoblots were performed as previously described (Crouch et al. Experimental Cell Research 2004; 292:274-87; Petty et al. Am J Pathol 2007; 170:1763-80) with the following modifications. Cultured cells were lysed in 2% SDS, 60 mM Tris, 10% glycerol containing 2× protease inhibitors (Complete, Roche, Indianapolis, Ind.) and quantitated using RC/DC Protein Assay (Bio-Rad, Hercules, Calif.). Primary tumor tissue (100 mg) was homogenized in 1 ml of 2% SDS lysis buffer, followed by incubation for 10 min. at 70° C. Tumor lysates were cleared by centrifugation for 10 min. at 14,000×g, 4° C. Lysates were boiled for 5 min. in the presence of 100 mM DTT and 0.01% bromophenol blue. Equal amounts of protein were loaded onto a 12% polyacrylamide gel and run at constant 200 V for 30-40 min. Gels were semi-dry transferred to polyvinylidene fluoride (PVDF) membranes and blocked in tris-buffered saline (TBS)-Tween (0.05%) containing 5% dry milk for one hour at room temperature. Endogenous Mig-7 protein was detected using our affinity-purified Mig-7 antibody at 0.16 μg/ml in TBS-T. After extensive washings, horseradish peroxidase (HRP)-labeled secondary anti-rabbit IgG antibody (Zymed Laboratories, San Francisco, Calif.) was used to detect the Mig-7 antibody at 0.038 μg/ml in TBS-T. Mouse anti-β-tubulin (clone AA2, Upstate, Inc., Lake Placid, N.Y.) was used at 0.2 μg/ml in TBS-T followed by extensive washing and incubation with HRP-labeled secondary anti-mouse IgG antibody (Cell Signalling, Danvers, Mass.). After stringent washings, Chemiluminescence Reagent (Amersham, Piscataway, N.J.) allowed detection of HRP-labeled antibodies when exposed to film.

For α2-macroglobulin assay blots, lysates were not reduced or boiled before electrophoresis and transferring to nitrocellulose membrane. MT1-MMP (MMP-14) was detected using a goat anti-human MMP-14 antibody (R&D Systems, Inc.) at 0.1 μg/ml and an HRP-labeled secondary anti-goat IgG antibody (Zymed Laboratories, San Francisco, Calif.) at 0.038 μg/ml.

Xenograft Nude Mouse Studies

Nude mouse studies were performed under Institutional Animal Care and Use Committee approval as previously described with modifications (Petty et al. Am J Pathol 2007; 170:1763-80; Phillips and Lindsey. Oncology Reports 2005; 13:37-44). Briefly, RL95 parental cells and cells stably transfected with siRNA expression vectors containing Mig-7 siRNA expression constructs 1-3, 3-1, or 4-2 G418 selected, pooled colonies were cultured as described previously (Petty et al. Am J Pathol 2007; 170:1763-80). Cells were lifted off the plate using 1× trypsin-EDTA (Gibco, Invitrogen), followed by inhibition of trypsin using Defined Trypsin Inhibitor (DTI) (Cascade Biologics, Portland, Oreg.). Viable cells were counted using trypan blue exclusion and a hemocytometer. For each injection, parental and siRNA 1-3, 3-1, or 4-2 expressing RL95 cell lines were suspended at 5×10⁵ each in serum-containing media with 10 ng/ml EGF (Gibco, Invitrogen) and 1:2 diluted Matrigel (>12 mg/ml, BD Biosciences) and injected subcutaneously into the dorsal neck region of nu/nu athymic mice (National Cancer Institute, Bethesda, Md.). The initial protein concentration of Matrigel was 10.3 mg/ml. Negative controls were mice injected with Matrigel alone (no cells). Five animals were injected per cell line. Tumor size was measured with a caliper every 2-3 days and volume was calculated using (length×width²)/2 as previously described (Williams et al. PNAS 2007; 104:2074-9). Mice were euthanized after 4 weeks.

Immunohistochemistry

Breast core punch biopsies on tissue array slides were obtained from Cybrdi, Inc (Frederick, Md.). Detection of Mig-7 protein was performed using Mig-7-specific affinity-purified antibody produced in rabbits injected with KLH-conjugated Mig-7 peptide (MAASRCSGL) representing the first nine amino acids of Mig-7 protein, as previously described (Petty et al. Am J Pathol 2007; 170:1763-80; Phillips and Lindsey. Oncology Reports 2005; 13:37-44). Briefly, 10% formalin fixed, paraffin embedded, 5 μm core tissue sections of normal breast or breast carcinomas as indicated were used. After deparaffinization with xylene for 10 minutes, rehydration through 100%, 95%, and 70% ethanol, antigen retrieval was performed for 2 seconds on ice in a microwave oven. Slides were washed two times in Dulbecco phosphate buffered saline (D-PBS) and then permeabilized with 0.01% digitonin (Sigma-Aldrich, St. Louis, Mo.) in PBS at room temperature for 30 minutes. After washing twice in PBS, slides were blocked in 10% horse serum (Gibco, Invitrogen) in D-PBS for 30 minutes at room temperature. Affinity-purified, polyclonal, rabbit anti-peptide (first nine amino acids of Mig-7) primary antibody (0.32 μg/μl), was diluted 1:50 and incubated on the tissue sections overnight at 4° C. Slides were washed twice in PBS then incubated for 20 minutes in 3% H₂O₂ in methanol. After washing twice in PBS, slides were incubated for 30 minutes with secondary antibody, goat anti-rabbit IgG coupled to horseradish peroxidase (HRP), in PBS containing 0.5% bovine serum albumin (BSA, Fisher Scientific). Slides were washed twice in PBS and then developed using 3,3′-diaminobenzidine (DAB) substrate (Vector Laboratories, Burlingame, Calif.) until specific brown staining was detected by microscopy (less than 3 minutes). After washing in water for 5 minutes, slides were counterstained in Hematoxylin QS (Vector Laboratories). Slides were dehydrated in 75%, 95%, and absolute ethanol, air dried, and coverslips mounted with DPX mounting medium (BDH Laboratory Supplies, Poole, UK). Secondary antibody alone or normal rabbit IgG instead of Mig-7 antibody served as controls. Images were taken on a Nikon Microphot microscope with a Retiga 2000R digital CCD camera.

RNA Isolation and RT-PCR

Total RNA from normal breast tissue and from breast carcinoma cell lines were purchased from Ambion, Inc. (Austin, Tex.). Isolation of total RNA from MCF-7 breast carcinoma cells, DNase treatment, and RT-PCR was performed as previously described (Crouch et al. Experimental Cell Research 2004; 292:274-87; Petty et al. Am J Pathol 2007; 170:1763-80; Phillips and Lindsey. Oncology Reports 2005; 13:37-44).

Peptides

| Peptides | Sequence |
|---|---|
| Control | MAASRCSGLYIVRNDTSG |
| | YIVRNDTSGLSGSQWVDS |
| | LSGSQWVDSPLKSPCQVW |
| Mig-7 specific | RVHMRACSAGSAYLKQMK |
| | GSAYLKQMKFCRMAASLD |
| | FCRMAASLDKVKKTDRGERG |
| MUC-1 control | GNNAPAHGVNNAPDNRPAP |

Control and Mig-7 specific peptides were synthesized by Biosource, Inc. (Wright et al. Journal of Immunotherapy 2000; 23:2-10). The MUC-1 control peptide was synthesized by American Peptide Co., Inc. Peptides, were at least 95% pure, evaluated by mass spectrometry, and solubilized in media.

Stimulation of Human Monocyte Cells and MCF-7 Killing Assays

Human peripheral blood MC isolation, culture, and stimulation were performed as previously described (Wright et al. Journal of Immunotherapy 2000; 23:2-10). Briefly, MCs were isolated from breast adenocarcinoma patients under Institutional Review Board (IRB) approval. Cells were cultured at 2×10⁶ cells/ml in AIMV® serum-free lymphocyte medium (Gibco, Invitrogen) in a 37° C. humidified 5% CO₂, 95% air atmosphere. IL-2 (Cetus, Inc., Emeryville, Calif.) was added twice per week at 100 IU/ml on days 0 and 4. Cells were stimulated with 1 μg/ml MUC-1 peptide alone or Mig-7 and MUC-1 peptides on days 0 and 7. MCF-7 cells (5×10³ per well) were plated into 96-well tissue culture plates. Effector peptide-stimulated MC were added to each well in two effector cell to target cell ratios (E:T) 10:1 and 5:1. Cell lysis was evaluated on day 8 of peptide stimulation using a tetrazolium salt XTT assay (Roche, Inc.) as previously described (Roehm et al. Journal of Immunological Methods 1991; 142:257-65). Treatments were in replicates of 6 and each experiment was performed at least twice.

Formation of formazan in XTT assays indicates viable cells. Formazan formed by target (MCF-7 cells) alone, effector (MC) alone, or background (no cells) was determined as the mean of six wells each. The percent specific lysis (% SL) was calculated as previously described (Wright et al. Journal of Immunotherapy 2000; 23:2-10):

$$\% SL = \frac{{OD}_{( target - medium )} - {OD}_{( experimental wells - well with corresponding number of effector )}}{{OD}_{( target - medium )}} \times 100$$

ELISA Cytokine Assay

TNF-α levels in media from each peptide treated monocyte cell culture were determined using the human BD OptEIA™ cytokine assay that is a solid phase sandwich Enzyme-Linked Immunosorbent Assay (ELISA) (BD Biosciences), according to manufacturer's instructions. Briefly, monocyte culture supernatant was added to TNF-α antibody coated wells and incubated for two hours. After washing, captured TNF-α was detected with a streptavidin-HRP conjugate mixed with a biotinylated anti-human TNF-α antibody. After washing, 3,3′, 5,5′-tetramethylbenzidine substrate was added and the reaction was stopped after development. Amounts of TNF-α were determined by measuring absorbance at 450 nm and comparison with a standard curve. Background of empty wells were subtracted before statistical analyses. Experiments were performed in replicates of six for each peptide treated monocyte experiment.

Statistical Analysis

Statistical significance of the in vitro cytotoxicity assays and of cytokine assays were determined by the Mann-Whitney Rank Sum test. Data from invasion assay and nude mouse xenograft assay were statistically analyzed by One-Way ANOVA and Tukey-Kramer post test and considered significant at p<0.05.

### Results

Antibody to Mig-7 Results in Decreased Invasion In Vitro.

Tumor cell invasion was quantitatively assessed in a transwell chemoinvasion assay (Hendrix et al. Invasion & Metastasis 1989; 9:278-97). HGF acts as a chemoattractant of HEC1A cells in transwell in vitro assays (Bae-Jumpet al. Gynecologic Oncology 1999; 73:265-72). A HEC1A cell line that expresses Mig-7 (Crouch et al. Experimental Cell Research 2004; 292:274-87) was treated with affinity-purified Mig-7 antibody directed against the first nine amino acids of full length Mig-7 protein to determine if this antibody could inhibit cell invasion.

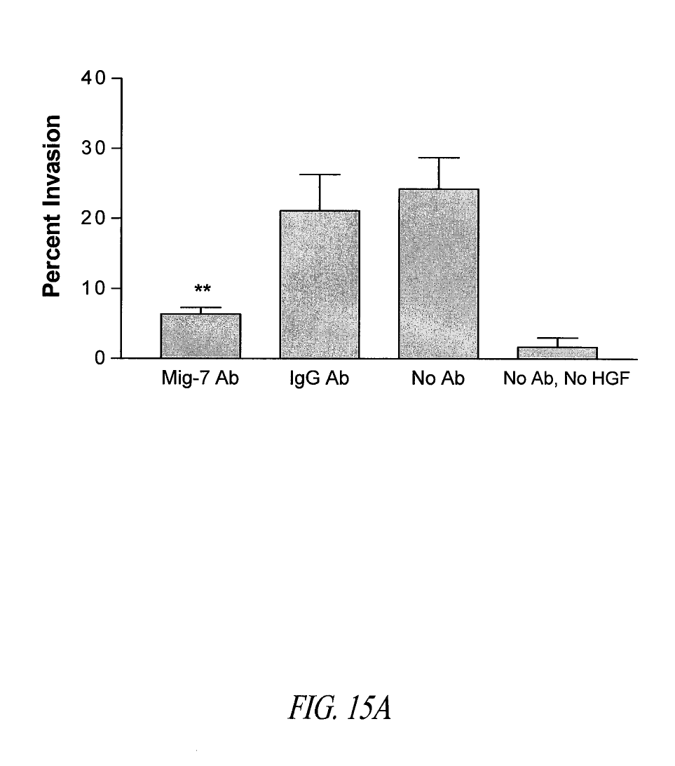

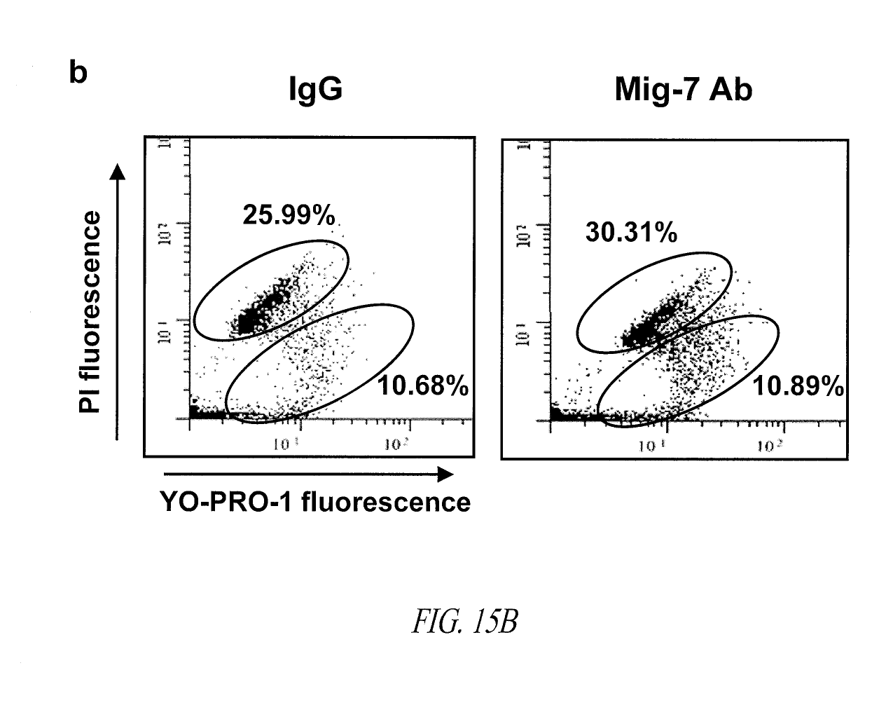

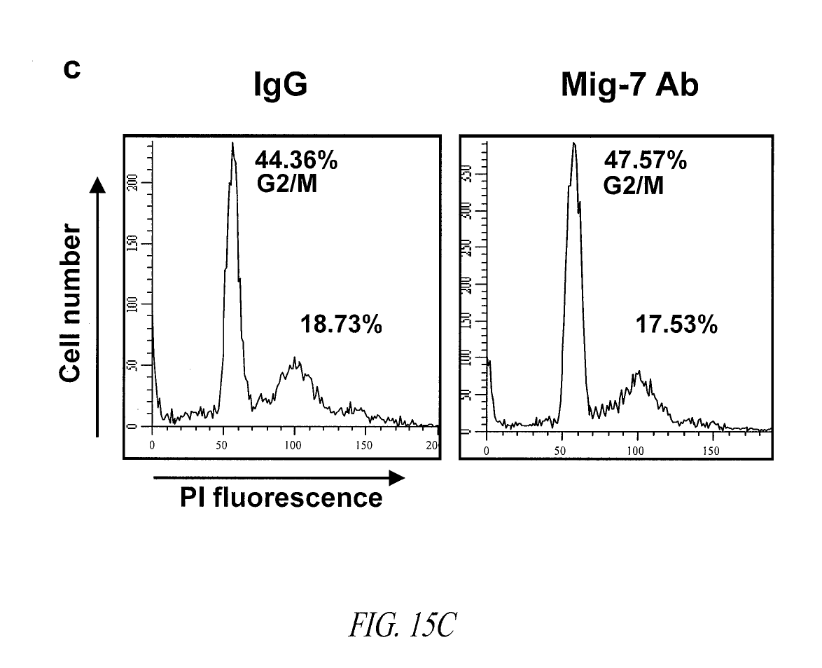

Mig-7 antibody treatment of HEC1A cells significantly decreased the average percentage of invaded cells counted as described in Methods and Materials section of this example. Chemoinvasion toward HGF by HEC1A cells was significantly (p value 0.0046) inhibited by 70% when treated with Mig-7 antibody when compared to normal rabbit IgG antibody treated cells (FIG. 15A). Furthermore, treatment with normal rabbit IgG antibody did not significantly inhibit HEC1A cell chemoinvasion when compared to no antibody treatment. No HGF chemoattractant reduced invasion of HEC1A cells by >90% (FIG. 15A). Flow cytometric analysis with the apoptotic dye YO-PRO-1 showed no significant increase in apoptosis due to Mig-7 antibody compared to normal rabbit IgG antibody treatment (FIG. 15B). In addition, flow cytometric cell cycle analysis using propidium iodide (PI) showed no significant decrease in cell proliferation of Mig-7 antibody-treated cells compared to untreated and control-treated cells (FIG. 15C).

Antibody or Expression of siRNA Specific to Mig-7 Decreases Activity of MT1-MMP

Mig-7 protein is primarily membrane-localized and is cysteine-rich (Crouch et al. Experimental Cell Research 2004; 292:274-87). Because a free thiol group on one of the many cysteine residues in membrane-localized Mig-7 activates MT1-MMP via the “cysteine switch” (Bescond et al. Biochemical and Biophysical Research Communications 1999; 263:498-503; Wart and Birkedal-Hansen. PNAS 1990; 87:5578-82) in the absence of proteolytic activation, we used the α2-macroglobulin capture assay to test for MT1-MMP activation. In addition, this test was used instead of zymography because the SDS in gel zymography activates the “cysteine switch” (Birkedal-Hansen and Taylo. Biochemical and Biophysical Research Communications 1982; 107:1173-8).

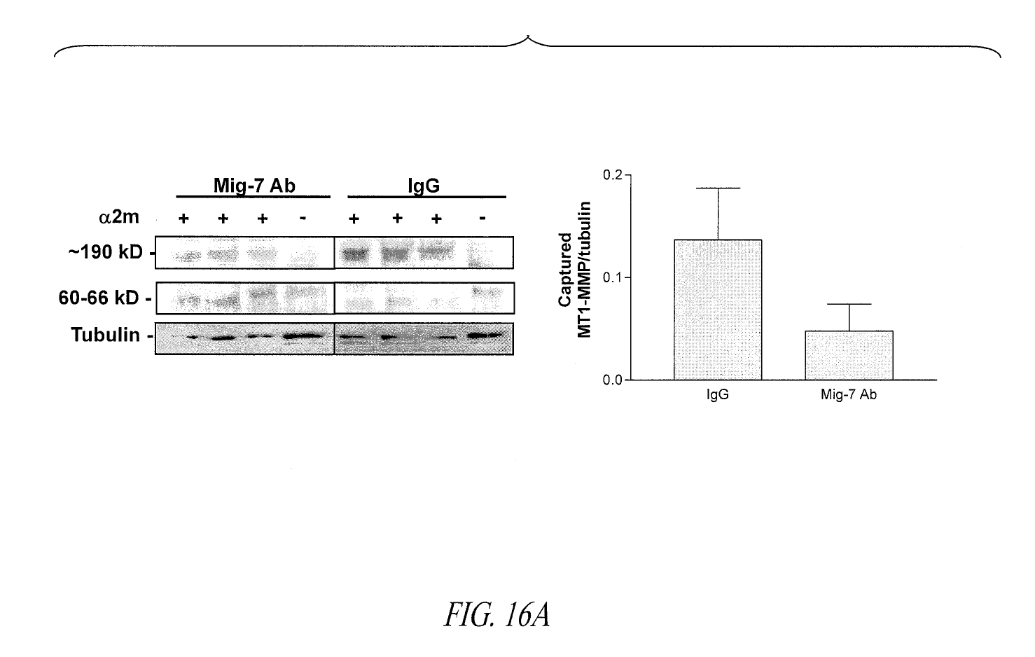

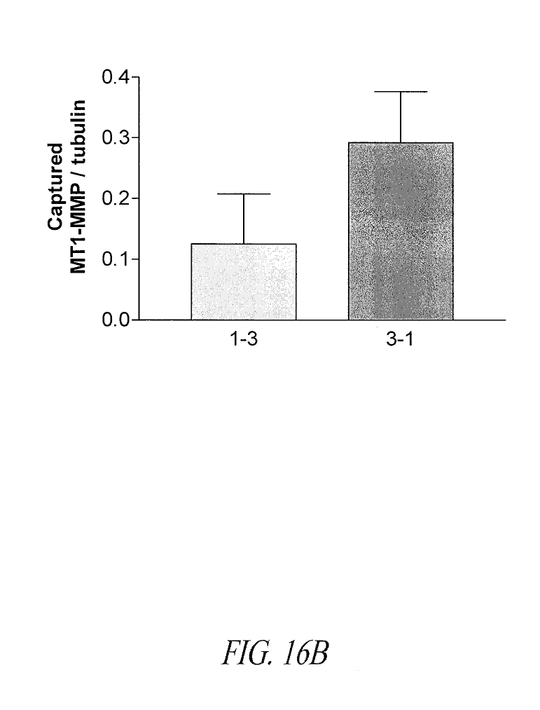

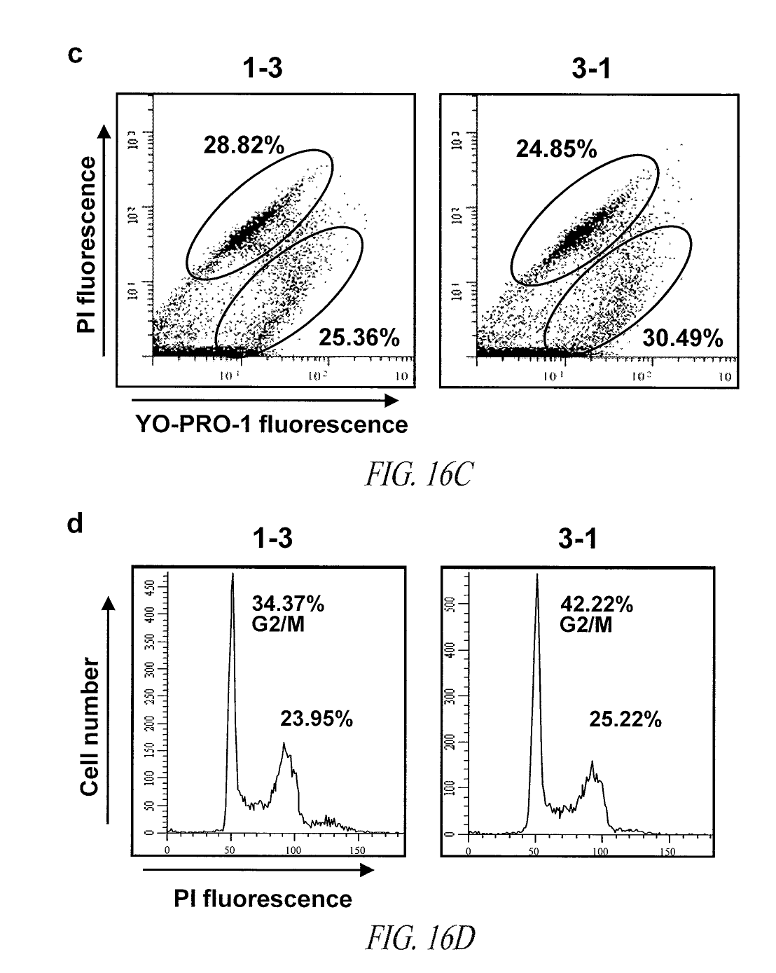

HEC1A cells treated with Mig-7 antibody showed a 54% decrease in the levels of activated MT1-MMP, as indicated by the upper, α2 macroglobulin captured band (˜190 kD), compared to cells treated with IgG control (FIG. 16A). The lower uncaptured, unactivated MT1-MMP band was 62 kD as indicated (FIG. 16A). Levels of activated MT1-MMP were determined by densitometry normalized with tubulin levels for each sample.

Mig-7 antibody treatment reduced levels of active MT1-MMP. RL95 endometrial carcinoma cells stably expressing siRNA constructs 1-3 and 3-1 were used to assay whether stable knockdown of Mig-7 expression in RL95 cells could inhibit MT1-MMP activation. Results from an α2-macroglobulin capture assay revealed that RL95 cells stably expressing Mig-7 siRNA 1-3 decreased Mig-7 protein levels by at least 50% and decreased levels of active MT1-MMP by 57% compared to control cells transfected with Mig-7 siRNA 3-1 that does not significantly reduce levels of Mig-7 protein (FIG. 16B). Analysis of these cell lines by flow cytometry for apoptosis and proliferation showed no significant differences between 1-3 and 3-1 cell lines (FIGS. 16C and D).

Expression of siRNA Specific to Mig-7 Decreases ERK1/2, Akt, and S6 Kinase Phosphorylation

A multiplex protein phosphorylation analysis was utilized to determine changes in protein activation exerted by Mig-7 chemoinvasion.

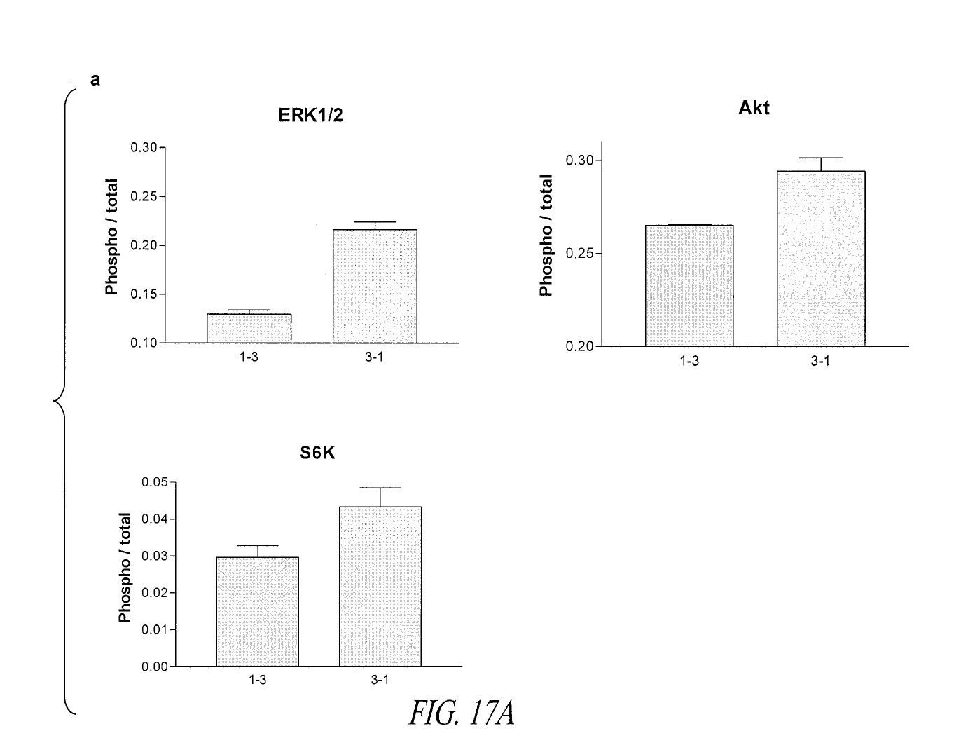

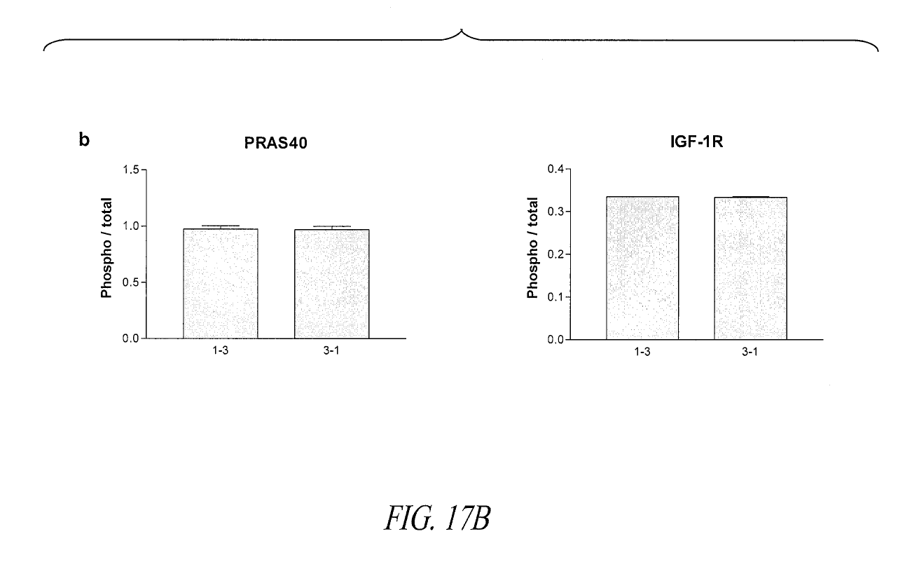

Phosphorylation status of proline-rich Akt substrate of 40 kilodaltons (PRAS40), ribosomal protein S6 kinase, extracellular signal-regulated kinase 1/2 (ERK1/2), insulin-like growth factor-1 receptor (IGF-1 R) and Akt (also known as protein kinase B) were analyzed. Decreased Mig-7 protein due to siRNA 1-3 expression decreased phosphorylation of AKT Ser⁴⁷³ by 10% (p<0.05), ERK1/2 Thr²⁰²/Tyr²⁰⁴, Thr¹⁸⁵/Tyr¹⁸⁷ by 40% (p<0.01), and S6 kinase Thr⁴²¹/Ser⁴²⁴ by 30% (p<0.05) compared to siRNA 3-1 expressing cells (FIG. 17A). No significant differences in phosphorylation between these two cell lines were detected for PRAS40 or for IGF-1R (FIG. 17B).

### Stimulation of Human Peripheral Blood Monocytes (MC) with Peptides Specific to Mig-7 Increases Levels of TNF and Increases Killing of MCF-7 Breast Carcinoma Cells In Vitro

Cancer immunotherapies include ex vivo tumor antigen stimulation of cancer patients' immune cells. Mig-7 peptides were used to stimulate MC and test their ability to increase killing of breast carcinoma cells in vitro. To test this hypothesis, we utilized our previously described method of MC isolated from two different breast cancer patients and the MCF-7 breast carcinoma cell line (Wright et al. Journal of Immunotherapy 2000; 23:2-10 Wright et al. International Journal of Molecular Medicine 2002; 9:401-4). Human MC were isolated under IRB approval as previously described (Wright et al. Journal of Immunotherapy 2000; 23:2-10). Our use of MCF-7 cells in the current study was warranted both by the fact that this system is optimized with this cell line (Wright et al. Journal of Immunotherapy 2000; 23:2-10), and that they expressed Mig-7 mRNA and protein (FIG. 18A).

Mig-7 peptides representing the +1 frameshifted protein sequence (Petty et al. GENE 2008; 414:49-59) (Accession DQ080207) or control peptides representing the sequence in the non-coding reading frame, i.e. the frame that did not produce protein (Petty et al. GENE 2008; 414:49-59), were used to determine sequences that contributed to enhanced MCF-7 killing by stimulated MC in vitro. In addition, these peptides included overlapping sequences to prevent the possible omission of a particular epitope. None of the peptides were significantly homologous to any sequence other than Mig-7 protein sequence or to translated cDNA sequences in databases available through the National Center for Biotechnology Information (NCBI). MUC-1 peptides served as internal controls because of their previous use and optimization in this assay (Wright et al. Journal of Immunotherapy 2000; 23:2-10; Wright et al. International Journal of Molecular Medicine 2002; 9:401-4).

Stimulation with Mig-7 peptides significantly enhanced MC killing of MCF-7 cells by >3-fold over MUC-1 peptide alone (0) or control (CTL) peptides. There was no significant difference between control peptides and MUC-1 peptide alone. An MC:MCF-7 cells ratio of 10:1 resulted in no difference between unstimulated and MUC-1-stimulated MC killing of MCF-7 cells. In contrast, at this ratio, Mig-7 peptides significantly increased MC killing of MCF-7 cells >2-fold over unstimulated and MUC-1-stimulated MC. At a 5:1 ratio, Mig-7 peptide stimulation of MC significantly enhanced their killing of MCF-7 cells 1.9-fold over MUC-1-peptide stimulation and at least 30-fold over no peptide stimulation. Mig-7 peptide stimulation also significantly increased levels of MC-produced TNF-α>3-fold over control peptide-stimulated MC as determined by ELISA.

Breast Carcinoma Tissue and Cells Express Mig-7

Mig-7 expression is detected in multiple types of tumor tissue and cells, but not in normal tissue and cells (Crouch et al. Experimental Cell Research 2004; 292:274-87; Phillips and Lindsey. Oncology Reports 2005; 13:37-44). Mig-7 specificity was determined in breast pre-cancerous states and carcinomas compared to normal breast tissue and cells. Relative RT-PCR was used to analyze total RNA as previously described (Crouch et al. Experimental Cell Research 2004; 292:274-87; Petty et al. Am J Pathol 2007; 170:1763-80; Phillips and Lindsey. Oncology Reports 2005; 13:37-44) from three breast carcinoma cell lines, T47D, MDA-MB453, DU4475, and from normal breast tissue from three subjects with no previous history of cancer. No amplification product specific to Mig-7 was detected in normal breast tissue RNA. However, all three breast carcinoma cell lines expressed Mig-7 mRNA (FIG. 18A).

Immunohistochemistry was performed on 70 human breast tissue arrayed core samples of normal, adenosis, papillomatosis, and carcinoma. The results showed that primarily carcinoma tissues (FIG. 18B) stained positive for Mig-7, whereas normal breast tissues lacked specific Mig-7 staining (FIG. 18C). No staining was detected with normal rabbit IgG antibody in breast carcinoma tissue serial section to that in FIG. 18B (FIG. 18D). A summary of the overall pathology report of Mig-7 staining for all 70 samples is as follows: normal: 0 positive, 3 negative; breast adenosis: 5 positive, 23 negative; breast papillomatosis: 0 positive, 3 negative; breast carcinoma: 19 positive, 17 negative. Pearson Chi-square analysis with 3 degrees of freedom to test goodness of Mig-7 fit for detection of breast carcinoma was statistically significant (p=0.008).

Stable Knockdown of Mig-7 Expression Reduces Primary Tumor Growth in Vivo

In addition to invasion, Mig-7 appears to play a role in the formation of vessel-like structures by tumor cells both in vitro and in vivo. The effect of stable Mig-7 knockdown through siRNA expression was tested on primary tumor growth in vivo.

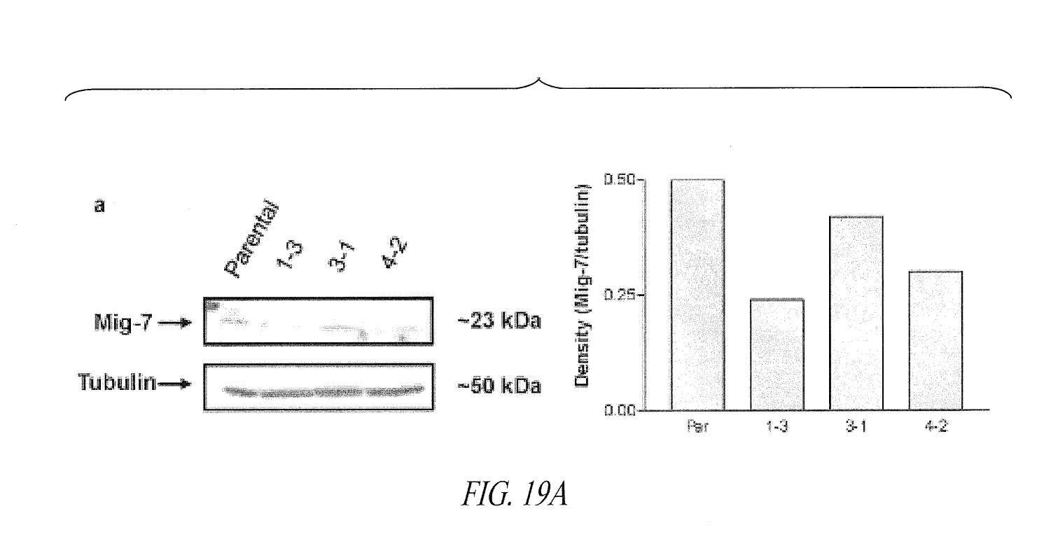

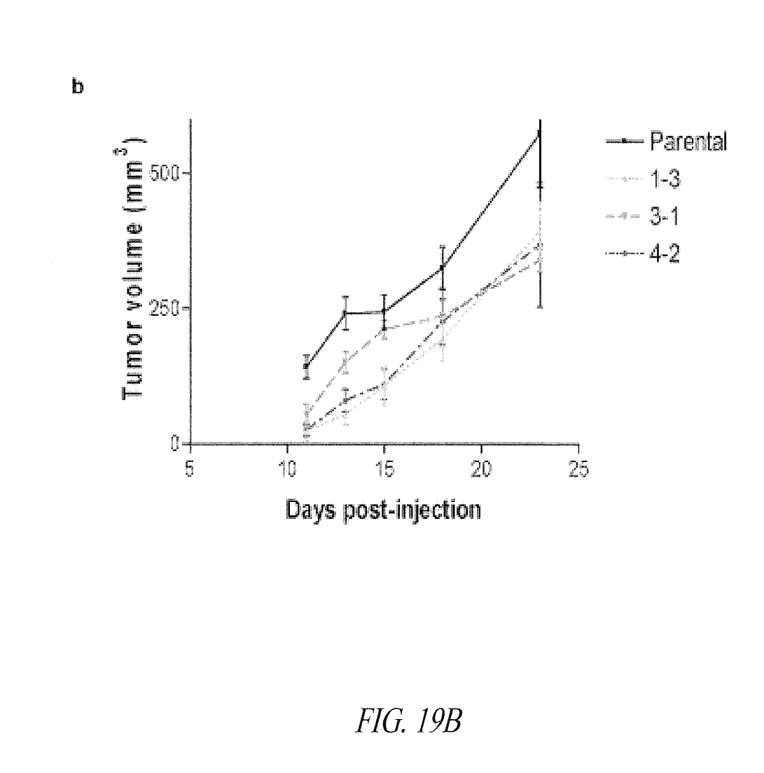

Parental or three RL95 endometrial carcinoma cell lines stably transfected with different Mig-7 siRNA expression plasmids, 1-3, 3-1, and 4-2 were each injected subcutaneously into nude mice and tumor growth measured as described in Methods and Materials. We confirmed that cells expressing siRNA 1-3 have reduced Mig-7 protein levels compared to parental and 3-1 expressing cells by Western blot and densitometry analyses (FIG. 19A). 1-3 expressing cells showed a 43% decrease in Mig-7 levels compared to 3-1 expressing cells. In addition, expression of a previously uncharacterized Mig-7 siRNA construct, 4-2, reduced Mig-7 protein levels by 30% compared to 3-1 expressing cells (FIG. 19A). Mice injected with RL95 cells expressing siRNA 1-3 and 4-2 showed significant (p<0.05) decreased tumor volume, 60% and 40-50%, respectively, 13 and 15 days after injection compared to mice injected with control cells expressing siRNA 3-1 (FIG. 19A). While tumor volume trended lower for siRNA expressing cells at days 18 and 23 this was not significantly different than parental cell line growth (FIG. 19B). Mice injected with Matrigel alone showed no tumor formation across the entire time period (data not shown).

The various aspects described above can be combined to provide further aspects. All of the U.S. patents, U.S. patent application publications, U.S. Patent applications, foreign patents, foreign patent applications and non-patent publications referred to in this specification and/or listed in the Application Data Sheet are incorporated herein by reference, in their entirety. Aspects of the aspects can be modified, if necessary to employ concepts of the various patents, applications and publications to provide yet further aspects.

These and other changes can be made to the aspects in light of the above-detailed description. In general, in the following claims, the terms used should not be construed to limit the claims to the specific aspects disclosed in the specification and the claims, but should be construed to include all possible aspects along with the full scope of equivalents to which such claims are entitled. Accordingly, the claims are not limited by the disclosure.

1. A composition comprising at least one siRNA expressed from a construct comprising a sequence selected from the group consisting of: SEQ ID NOs: 43-49.

2. The composition of claim 1, wherein the at least one siRNA is double stranded.

3. A method for inhibiting Mig-7 expression in a cell comprising contacting said cell with an effective amount of Mig-7 inhibitor, wherein said inhibitor comprises siRNA.

4. The method of claim 3 wherein said cell is located in a subject.

5. The method of claim 4 wherein said subject is a human.

6-15. (canceled)
